(19) United States
(12) Patent Application Publication
(10) Pub. No.: US 2020/0290950 A1
(43) Pub. Date: Sep. 17, 2020

(54) ALKAMIDE COMPOUNDS AND USES THEREOF

(71) Applicant: NORTH CAROLINA STATE UNIVERSITY, Raleigh, NC (US)

(72) Inventors: Scott M. LASTER, Raleigh, NC (US); Travis V. GULLEDGE, Raleigh, NC (US); Joshua G. PIERCE, Raleigh, NC (US); Wolfgang BAEUMER, Berlin (DE); Santosh K. MISHRA, Raleigh, NC (US)

(21) Appl. No.: 16/753,060

(22) PCT Filed: Oct. 5, 2018
(86) PCT No.: PCT/US2018/054554

§ 371 Date: Apr. 2, 2020

Related U.S. Application Data
(60) Provisional application No. 62/568,427, filed on Oct. 5, 2017.

(51) Int. Cl. C07C 233/09; C07C 233/11; C07C 233/05

(52) U.S. Cl. CPC C07C 233/09; C07C 233/05; C07C 233/11

(57) ABSTRACT

The present disclosure relates to alkamide compounds and compositions for treating allergic diseases, pain, or itch.

### CROSS REFERENCE TO RELATED APPLICATIONS

This application claims the benefit of U.S. Provisional Patent Application Ser. No. 62/568,427 filed Oct. 5, 2017, the disclosure of which is expressly incorporated herein by reference.

### FIELD

The present disclosure relates to alkamide compounds and compositions for treating allergic diseases, pain, or itch.

### BACKGROUND

Allergic diseases impact millions of people and the prevalence of these disorders continues to rise in the United States and other developed countries. Up to 20% of the population can suffer from allergy. Allergic disease can affect a variety of organs and tissues including the skin (hypersensitive reactions), lungs and respiratory tract (asthma and rhinitis), and gastrointestinal tract (food allergies and irritable bowel syndrome). Mast cells, found in mucosal and serosal tissues, contain large, dense granules filled with pre-formed mediators and are critical to allergic responses. Basophils are also important to allergic disease and share many features with mast cells including mediator-containing granules and high affinity IgE receptors (FcεRI). Mast cells and basophils exocytose their granules releasing histamine, cytokines/chemokines, proteases, and growth factors into the surrounding tissue causing the symptoms of allergic disease. In addition to the release from granules, mast cells and basophils can also produce cytokines, chemokines, prostaglandins, and leukotrienes by de novo biosynthetic pathways.

Alkamides, also known as fatty acid amides (or sometimes referred to as alkylamides), are a class of fatty-acid-like molecules whose activity has been linked to the anti-inflammatory activity of Echinacea extracts. Alkamides are produced by a number of medicinal plants including Echinacea spp., Acmella oleracea, and Zanthoxylum americanum. Each alkamide contains an amide group, a hydrocarbon chain, and a functional group (for example, isobutyl, benzyl, or methyl group). The number of carbons in the alkyl chain can vary (typically about 10-20 carbons), as well as the number and position of double and triple bonds. Alkamide structures can be further diversified with modifications including hydroxylations and methylations. Certain plant species can produce multiple alkamides. For example, over 18 different alkamides have been identified from E. purpurea. Alkamides are capable of suppressing cytokine, chemokine, and prostaglandin production from a number of immune cell types including monocytes, macrophages, and T cells. Thus, alkamides can contribute to the anti-inflammatory, medicinal activity of these plants.

Allergic reactions are highly dependent on the activity of mast cells. Unfortunately, current treatments for allergy are either ineffective, targeting only one of the many mediators released by mast cells, or display dangerous side effects.

The compounds, compositions, and methods disclosed herein address these and other needs.

### SUMMARY

Disclosed herein are alkamide compounds and compositions for treating allergic diseases, pain, or itch. The inventors identified alkamide compounds which inhibit the release of factors (e.g., β-hexosaminidase and histamine) from mast cells. The alkamide compounds can block calcium influx in mast cells and other cell types, which modulates an array of pathological responses. Further, the alkamide compounds herein can inhibit neuronal cell receptors which mediate responses to environmental stimuli including pain (e.g., heat, cold). The alkamide-containing compositions can also be used in, for example, a topical agent, to reduce the itch response.

In some aspects, disclosed herein is a method of treating pain or itch, comprising administering to a subject in need thereof, a therapeutically effective amount of a compound of Formula I:

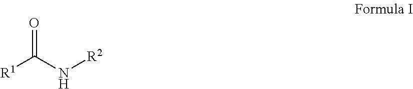

wherein: R¹ and R² are independently selected from unsubstituted alkyl or substituted alkyl, unsubstituted alkenyl or substituted alkenyl, unsubstituted arylalkyl or substituted arylalkyl; or a pharmaceutically acceptable salt thereof.

In some embodiments, R¹ is selected from unsubstituted arylalkyl and R² is selected from unsubstituted alkyl or substituted alkyl.

In some embodiments, R¹ is unsubstituted alkyl. In some embodiments, R¹ is an unbranched alkyl. In some embodiments, R¹ is a C₁₁ alkyl. In some embodiments, R¹ is a haloalkyl.

In some embodiments, R² is a branched alkyl. In some embodiments, R² is isobutyl. In some embodiments, R² is a haloalkyl.

In additional aspects, disclosed herein is a method of treating an allergic disease, comprising administering to a subject in need thereof, a therapeutically effective amount of a compound of Formula I:

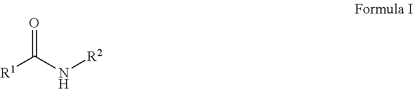

wherein R¹ and R² are independently selected from unsubstituted alkyl or substituted alkyl, unsubstituted alkenyl or substituted alkenyl, unsubstituted arylalkyl or substituted arylalkyl; or a pharmaceutically acceptable salt thereof.

In some embodiments, R¹ is selected from unsubstituted arylalkyl and R² is selected from unsubstituted alkyl or substituted alkyl.

In some embodiments, R¹ is unsubstituted alkyl. In some embodiments, R¹ is an unbranched alkyl. In some embodiments, R¹ is a C₁₁ alkyl. In some embodiments, R¹ is a haloalkyl.

In some embodiments, R² is a branched alkyl. In some embodiments, R² is isobutyl. In some embodiments, R² is a haloalkyl.

In some embodiments, the allergic disease is selected from a hypersensitive reaction of the skin to a metal, an allergic response to pollen, an allergic response to pet dander, eczema, urticaria, asthma, or a food allergy.

In some aspects, disclosed herein is a compound of Formula I:

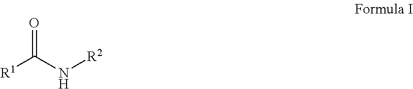

wherein: R¹ is selected from unsubstituted arylalkyl, substituted arylalkyl, or substituted alkyl; R² is selected from unsubstituted alkyl or substituted alkyl; or a pharmaceutically acceptable salt thereof.

### BRIEF DESCRIPTION OF THE DRAWINGS

The accompanying figures, which are incorporated in and constitute a part of this specification, illustrate several aspects described below.

FIGS. 1A-1F. A15 inhibits BMMC and RBL-2H3 cell degranulation. The structure of alkamide A15 is shown with different functional groups and carbons in the fatty acid chain labeled (FIG. 1A). BMMCs were plated and stimulated 4 hrs later with 1 μM A23187 and the indicated concentrations of A15 or vehicle only. The percent degranulation was measured after 1 hr (FIG. 1B). RBL-2H3 cells were treated with A23187 and the percent degranulation was determined 1 hr later (FIG. 1C) or every 10 min over a 1 hr time course (FIG. 1D). RBL-2H3 cells were plated and incubated for 4 hrs before the media was aspirated and replaced with media containing 1 μg/mL IgE anti-DNP. Cells were incubated overnight and washed prior to stimulation. The percent degranulation from RBL-2H3 cells was calculated after stimulation with 50 ng/mL DNP-HSA in combination with the indicated concentrations of A15 or vehicle only after 1 hr (FIG. 1E). RBL-2H3 cells were stimulated for 1 hr in Tyrode's buffer before supernatants were collected and levels of histamine were measured using a competitive inhibition EIA kit (FIG. 1F). Data shown are the means±SEM from 3-4 independent experiments (FIG. 1B, FIG. 1C and FIG. 1F) or the means±SEM of triplicate wells from a representative experiment from 3 independent experiments (FIG. 1D and FIG. 1E). Statistical analysis was performed using a repeated measures one-way ANOVA with Dunnett's post-hoc test, *p<0.05, **p<0.01, ***p<0.001.

FIG. 2. A15 inhibits granule release from BMMCs. BMMCs were treated with 100 μM A15 or 0.25% ethanol only (vehicle) alone and in combination with 1 μM A23187 for 30 min. BMMCs were cytocentrifuged onto a glass slide, fixed, stained with toluidine blue (1%, pH<1) for 30 min, and washed before observing microscopically. Images were acquired at 400× magnification and are representative.

FIGS. 3A-3H. A15 inhibits calcium influx in RBL-2H3 cells and BMMCs. RBL-2H3 cells (FIGS. 3A-3D) and BMMCs (FIGS. 3E-3H) were plated and incubated overnight prior to treatments. For DNP-BSA treatments, overnight incubation also included 1 μg/mL IgE anti-DNP. Prior to stimulation, cells were washed and loaded with the fluorescent, calcium-sensitive dye fluo-4 AM using the Fluo-4 AM Direct™ Calcium Assay Kit and fluorescence was measured on a microplate reader. Calcium influx was triggered in all experiments using either 1 μM A23187 or 50 ng/mL DNP-BSA, as indicated. Bar graphs represent the means±SEM of the Δ in RFU's at 1 min post-A23187- or DNP-BSA-stimulation from 3 independent experiments (FIGS. 3B, 3D, 3F, 3H). Statistical analysis was performed using a one-way ANOVA with Dunnett's post-hoc test, *p<0.05, **p<0.01, ***p<0.001.

FIGS. 4A-4D. A15 inhibits A23187-stimulated calcium influx in RAW 264.7 and Jurkat cells. RAW 264.7 (FIG. 4A-4B) or Jurkat cells (FIG. 4C-4D) were loaded with fluo-4 AM using the Fluo-4 AM Direct™ Calcium Assay Kit and fluorescence was measured on a microplate reader. Cells were stimulated with 1 μM A23187 in combination with vehicle only or 100 μM A15. Bar graphs represent the means±SEM of the Δ in RFU's at 1 min post-A23187 stimulation from 3 independent experiments. Statistical analysis was performed using an unpaired Student's t-test, *p<0.05.

FIGS. 5A-5D. Alkamide-containing Echinacea purpurea extracts and fractions inhibit RBL-2H3 cell degranulation and calcium influx. A flow chart summarizing how fractions with high and low alkamide content were prepared from an ethanolic extract of E. purpurea roots (FIG. 5A). RBL-2H3 cells were plated and incubated overnight prior to stimulation. Percent degranulation was measured 1 hr after stimulation with 1 μM A23187 in combination with vehicle only or 25 μg/mL of the indicated E. purpurea samples. (FIG. 5B). RBL-2H3 cells were loaded with fluo-4 AM using the Fluo-4 AM Direct™ Calcium Assay Kit and fluorescence was measured on a microplate reader. Cells were stimulated with 1 μM A23187 in combination with the indicated E. purpurea samples (FIGS. 5C and 5D). EE=ethanolic E. purpurea extract, CL=chloroform layer, F2, F6, F8=Fraction 2, Fraction 6 and Fraction 8, respectively. Results are displayed as the means±SEM from 3 independent experiments (FIGS. 5B and 5D) or the means±SEM of triplicate wells from a representative experiment from 3 independent experiments (FIG. 5C). Statistical analysis was performed using a repeated measures one-way ANOVA with Dunnett's post-hoc test, *p<0.05, **p<0.01, ***p<0.001. ^{†}EE was tested at 50 μg/mL.

FIGS. 6A-6B. A15 inhibits de novo production of TNF-α and PGE₂ from RBL-2H3 cells. RBL-2H3 cells were plated and incubated overnight prior to stimulation. Cells were stimulated with 1 μM A23187 in combination with the indicated doses of A15. Supernatants were collected 8 hrs later and TNF-α (FIG. 6A) or PGE₂ (FIG. 6B) levels were measured using ELISA kits. Data shown are the means±SEM from 3 independent experiments. Statistical analysis was performed using a one-way ANOVA with Dunnett's post-hoc test, *p<0.05, **p<0.01, ***p<0.001.

FIG. 7. A15 is not cytotoxic to RBL-2H3 cells. RBL-2H3 cells were plated and incubated overnight prior to stimulation. Cells were treated overnight with the indicated concentrations of A15 10 μg/mL cycloheximide as a positive control. Supernatants were collected and analyzed for LDH release using the Pierce LDH Cytotoxicity Kit. Data are displayed as the means±SD of duplicate wells from a representative experiment out of 2 independent experiments. Statistical analysis was performed using a one-way ANOVA with Dunnett's post-hoc test, ***p<0.001.

FIG. 8. A15 does not inhibit β-hexosaminidase enzyme activity. RBL-2H3 cells were lysed with 0.1% Triton X-100 to release β-hex from the cells. Lysates were incubated for 1 hr at 37° C. with the substrate, p-NAG, in combination with the indicated concentrations of A15 or vehicle only. The absorbance at 405 nm was measured using a microplate spectrophotometer. Results shown are the means±SEM of triplicate wells from a representative experiment out of 2 independent experiments. Statistical analysis was performed using a one-way ANOVA with Dunnett's post-hoc test.

FIG. 9. A15 inhibits calcium influx in RBL-2H3 cells. Prior to stimulation, cells were washed and loaded with the fluorescent, calcium-sensitive dye fluo-4 AM using the Fluo-4 AM Direct™ Calcium Assay Kit. Cells were treated with 100 μM A15 (D, E, F), 0.25% ethanol only (vehicle) (A, B, C), 1 μM A23187 (G, H, I), and 100 μM A15 in combination with 1 μM A23187 (J, K, L) as indicated. Images were acquired at 30, 60, and 120 seconds, post-stimulation. Images are representative of several experiments.

FIG. 10. The structures of alkamides A15 and YM8-85.

FIG. 11. Calcium response from DRG sensory neurons to alkamide of transient receptors. Mustard a TRPA1-receptor agonist activates DRG neurons in gray (A) and capsaicin, an agonist for TRPV1-receptors in the DRG neurons in red (B). Ligand A15 inhibits both TRPA1 and TRPV1 receptors activation in orange; however, 85Y a synthetic ligand only reduces the inhibition of TRPA1 but not TRPV1 receptors in blue. Total number of neurons (n)=135-145; p≤0.05.

FIG. 12. Antinociceptive effect of topical administration of alkamide A15. Measured paw withdrawal latency to Hargreave's (A) cold assay using dry ice (B) and touch using von-Frey (C). Data were analyzed using parametric evaluation (AUC). Bars represent the mean SEM, n≥, *Significantly different (p≤0.05).

FIG. 13. The effects of A15 on toluene-2,4-diisocyanate (TDI) ear swelling. Replicate experiments are shown in panels A and B.

FIG. 14. Scratching bouts in toluene-2,4-diisocyanate (TDI) treated mice.

FIG. 15. Structures of constituents produced by E. purpurea.

FIGS. 16A-16B. Time courses of A23187 and DNP-BSA induced degranulation from RBL-2H3 cells. RBL-2H3 cells were plated overnight then stimulated with 1 μM A23187 (FIG. 16A) or 5 ng/mL DNP-BSA (IgE-DNP) (FIG. 16B) for indicated times. Graphs display the mean percent β-hex released from the cell, as indicated in the Materials and Methods section. Experiments were performed in duplicate.

FIGS. 17A-17B. Concentration dependence of A23187 and DNP-BSA induce degranulation in RBL-2H3 cells. RBL-2H3 cells were plated overnight then stimulated with different concentrations of A23187 (FIG. 17A) or 5 ng/mL DNP-BSA (IgE-DNP) (FIG. 17B) for 1 h. Graphs display the mean total percent β-hexosaminidase released from the cell. Experiments were performed in duplicate.

FIGS. 18A-18B. Inhibition of calcium influx by TMB-8 in RBL-2H3 cells. RBL-2H3 cells were plated overnight, incubated with Fluo-4 AM for 1 h, stimulated with 1 μM A23187 (FIG. 18A) or 5 ng/mL DNP-BSA (FIG. 18B) in the absence (solid line) or presence (dashed line) of TMB-8 and the fluorescence was read every 5 secs for 2 mins. Data are presented as the mean change in RFU. Experiments were performed in triplicate.

FIG. 19. Inhibition of degranulation by TMB-8 in RBL-2H3 cells. RBL-2H3 cells were plated overnight, stimulated with 1 μM A23187 (open) or 5 ng/mL DNP-BSA (gray) for 1 h in the absence or presence of TMB-8. Graphs display the mean percent β-hex release. Experiments were performed in triplicate. **P<0.05 (unpaired t test). Mean±s.e.m

FIGS. 20A-20G. Structures of alkamides with varying fatty acid lengths. YM11-55 (FIG. 20A) A15 (FIG. 20B), YM5-27 (FIG. 20C), YM8-86 (FIG. 20D), YM8-85 (FIG. 20E), YM5-47 (FIG. 20F), YM8-87 (FIG. 20H). The number in parenthesis represents the number of carbons in the fatty acid chain.

FIGS. 21A-21D. The inhibition of degranulation is dependent on the length of the alkamide fatty acid chain. RBL-2H3 cells were plated overnight, stimulated with 1 μM A23187 (FIG. 21A) or 5 ng/mL DNP-BSA (FIG. 21B) for 1 h in the absence or presence of each compound. Graphs display the mean percent β-hex release. Data are presented as the mean of 4 experiments. Percent inhibition of degranulation of A23187 (FIG. 21C) or DNP-BSA (FIG. 21D) using data from A and B, respectively. *P<0.05, **P<0.01, ***P<0.001, one-way ANOVA with Dunnett's post hoc test compared to vehicle (A, B) or A15 (C, D).

FIGS. 22A-22D. The alkamide fatty acid is necessary for inhibition of calcium influx. RBL-2H3 cells were plated overnight, incubated with Fluo-4 AM for 1 h, stimulated by 1 μM A23187 (FIG. 22A) or 5 ng/mL DNP-BSA (FIG. 22B) in the absence or presence of each alkamide, and fluorescence was read for 2 mins every 5 secs. Data are presented as the mean change in RFU of 4 experiments. Percent inhibition of calcium influx was calculated for A23187 (FIG. 22C) and DNP-BSA (FIG. 22D) stimulated cells at 2 mins. *P<0.05, **P<0.01, and ***P<0.001 one-way ANOVA with Dunnett's post hoc test compared to A15.

FIG. 23A-23B. The relationship between the length of the alkamide fatty acid, inhibition of degranulation and calcium influx. Correlation between length of fatty acid chain structure versus percent inhibition of degranulation or calcium release (See FIGS. 21 and 22). Data was fit using the second order polynomial fit test. Data are based on a mean of 4 experiments. YM8-87, which has the least carbons in the fatty acid, and YM11-55, which has the most carbons in the fatty acid, were weak inhibitors of degranulation and calcium in A23187 (FIG. 23A) and DNP-BSA (FIG. 23B) stimulated cells.

FIGS. 24A-24B. A23187 induces rapid production of TNF-α at low concentrations in RBL-2H3 cells. RBL-2H3 cells were plated overnight and stimulated with 1 μM A23187 for indicated timepoints over 18 h (FIG. 24A) or varying concentrations of A23187 for 18 h (FIG. 24B). The concentration of TNF-α was analyzed in the supernatants by ELISA. Experiments were performed in duplicate in A. A representative of 1 experiment is shown in B.

FIGS. 25A-25B. A15 strongly inhibits production of TNF-α. RBL-2H3 cells were plated overnight and stimulated with 1 μM A23187 in the absence or presence of each alkamide for 18 h. Culture supernatants were analyzed for TNF-α levels by ELISA and experiments were performed in triplicate (FIG. 25A). Percent inhibition of TNF-α secretion for each alkamide listed in FIG. 25B. *P<0.05, **P<0.01, ***P<0.001, one-way ANOVA with Dunnett's post hoc test compared to vehicle (FIG. 25A) or A15 (FIG. 25B).

FIG. 26. The relationship between the length of the alkamide fatty acid, inhibition of degranulation and TNF-α release. Correlation of fatty acid chain between percent inhibition of degranulation and TNF-α secretion (See FIGS. 21 and 25) in 1 μM A23187 stimulated RBL-2H3 cells.

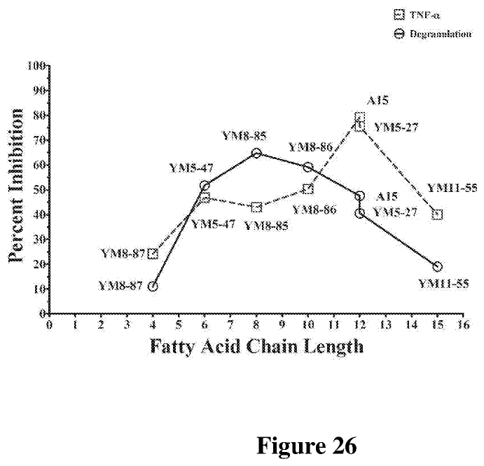

### DETAILED DESCRIPTION

Disclosed herein are alkamide compounds and compositions for treating allergic diseases, pain, or itch. The inventors identified alkamide compounds which inhibit the release of factors (e.g., β-hexosaminidase and histamine) from mast cells. The alkamide compounds can block calcium influx in mast cells and other cell types, which modulates an array of pathological responses. Further, the alkamide compounds herein can inhibit neuronal cell receptors which mediate responses to environmental stimuli including pain (e.g., heat, cold). The alkamide-containing compositions can also be used in, for example, a topical agent, to reduce the itch response.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

The following definitions are provided for the full understanding of terms used in this specification.

### Terminology

As used herein, the article “a,” “an,” and “the” means “at least one,” unless the context in which the article is used clearly indicates otherwise.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group. In some embodiments, the alkyl comprises 1 to 10 carbon atoms, such as methyl, ethyl, n-propyl, isopropyl, n-butyl, isobutyl, t-butyl, pentyl, hexyl, heptyl, octyl, nonyl, decyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like. This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” or “CO” is a short hand notation for C═O.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “amide” or “carboxanmide” refers to a group of the formula —C(O)NZ¹Z² where Z¹ and Z² can be, independently, an alkyl, halogenated alkyl, alkenyl, alkynyl, acyl, aryl, heteroaryl, cycloalkyl, cycloalkenyl, heterocycloalkyl, or heterocycloalkenyl group described above; or together with the nitrogen to which they are bonded, Z¹ and Z² can form a heterocyclic ring.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxyl group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, and methods, examples of which are illustrated in the accompanying Examples and Figures.

### Compounds and Methods

Disclosed herein is a method of treating an allergic disease, comprising administering to a subject in need thereof, a therapeutically effective amount of a compound of Formula I:

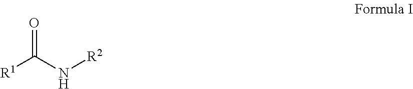

wherein R¹ and R² are independently selected from unsubstituted alkyl or substituted alkyl, unsubstituted alkenyl or substituted alkenyl, unsubstituted arylalkyl or substituted arylalkyl; or a pharmaceutically acceptable salt thereof.

In some embodiments, the allergic disease is selected from a hypersensitive reaction of the skin to a metal, an allergic response to pollen, an allergic response to pet dander, eczema, urticaria, asthma, or a food allergy.

Also disclosed herein is a method of treating pain or itch, comprising administering to a subject in need thereof, a therapeutically effective amount of a compound of Formula I:

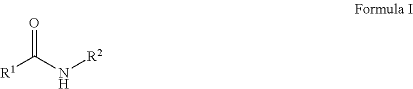

wherein: R¹ and R² are independently selected from unsubstituted alkyl or substituted alkyl, unsubstituted alkenyl or substituted alkenyl, unsubstituted arylalkyl or substituted arylalkyl; or a pharmaceutically acceptable salt thereof.

In some embodiments, R¹ is selected from unsubstituted arylalkyl and R² is selected from unsubstituted alkyl or substituted alkyl.

In some embodiments, R¹ is unsubstituted alkyl. In some embodiments, R¹ is substituted alkyl. In some embodiments, R¹ is an unbranched alkyl. In some embodiments, R¹ is a C₁₁alkyl. In some embodiments, R¹ is a C₃₋₁₅ alkyl. In some embodiments, R¹ is a C₃alkyl, C₄alkyl, C₅alkyl, C₆alkyl, C₇alkyl, C₅alkyl, C₉alkyl, C₁₀alkyl, C₁₁alkyl, C₁₂alkyl, C₁₃alkyl, C₁₄alkyl, or C₁₅alkyl. In some embodiments, R¹ is a haloalkyl.

In some embodiments, R¹ is unsubstituted alkenyl. In some embodiments, R¹ is substituted alkenyl. In some embodiments, R¹ is a C₁₁alkenyl. In some embodiments, R¹ is a C₃₋₁₅alkenyl. In some embodiments, R¹ is a C₃alkenyl, C₄alkenyl, C₅alkenyl, C₆alkenyl, C₇alkenyl, C₈alkenyl, C₉alkenyl, C₁₀alkenyl, C₁₁alkenyl, C₁₂alkenyl, C₁₃alkenyl, C₁₄alkenyl, or C₁₅alkenyl. In some embodiments, R¹ is unsubstituted arylalkyl. In some embodiments, R¹ is substituted arylalkyl.

In some embodiments, R² is a branched alkyl. In some embodiments, R² is isobutyl. In some embodiments, R² is isopentyl. In some embodiments, R² is a haloalkyl.

In some embodiments, R² is a branched alkyl, wherein the branched alkyl is substituted with aryl, halogen, haloalkyl, or arylalkyl. In some embodiments, R² is alkyl, wherein the alkyl is substituted with haloalkyl. In some embodiments, R² is benzyl.

In some embodiments, R² is unsubstituted alkyl. In some embodiments, R² is substituted alkyl. In some embodiments, R² is an unbranched alkyl. In some embodiments, R² is a C₁₁alkyl. In some embodiments, R² is a C₃₋₁₅ alkyl. In some embodiments, R² is a C₃alkyl, C₄alkyl, C₅alkyl, C₆alkyl, C₇alkyl, C₈alkyl, C₉alkyl, C₁₀alkyl, C₁₁alkyl, C₁₂alkyl, C₁₃alkyl, C₁₄alkyl, or C₁₅alkyl. In some embodiments, R² is a haloalkyl.

In some embodiments, R² is unsubstituted alkenyl. In some embodiments, R² is substituted alkenyl. In some embodiments, R² is a C₁₁alkenyl. In some embodiments, R² is a C₃₋₁₅ alkenyl. In some embodiments, R² is a C₃alkenyl, C₄alkenyl, C₅alkenyl, C₆alkenyl, C₇alkenyl, C₈alkenyl, C₉alkenyl, C₁₀alkenyl, C₁₁alkenyl, C₁₂alkenyl, C₁₃alkenyl, C₁₄alkenyl, or C₁₅alkenyl. In some embodiments, R² is unsubstituted arylalkyl. In some embodiments, R² is substituted arylalkyl.

In some aspects, disclosed herein is a compound of Formula I:

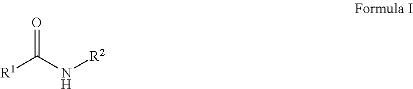

wherein: R¹ is selected from unsubstituted arylalkyl, substituted arylalkyl, or substituted alkyl; R² is selected from unsubstituted alkyl or substituted alkyl; or a pharmaceutically acceptable salt thereof.

In some aspects, disclosed herein is a compound of Formula I:

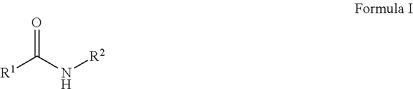

wherein: R¹ is selected from unsubstituted alkyl or substituted alkyl; R² is selected from alkyl, wherein the alkyl is substituted with aryl, halogen, haloalkyl, or arylalkyl; or a pharmaceutically acceptable salt thereof.

In some embodiments, disclosed herein is a compound of Formula Ia:

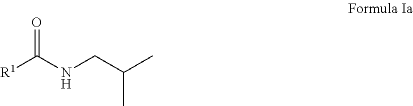

wherein: R¹ is selected from unsubstituted arylalkyl, substituted arylalkyl, or substituted alkyl; or a pharmaceutically acceptable salt thereof.

In some aspects, disclosed herein is a compound of Formula II:

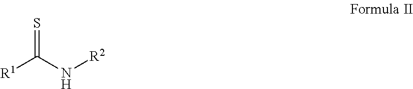

wherein: R¹ is selected from unsubstituted arylalkyl, substituted arylalkyl, or substituted alkyl; R² is selected from unsubstituted alkyl or substituted alkyl; or a pharmaceutically acceptable salt thereof.

In some aspects, disclosed herein is a compound of Formula IIa:

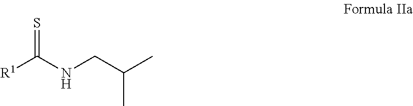

wherein: R¹ is selected from unsubstituted arylalkyl, substituted arylalkyl, or substituted alkyl; or a pharmaceutically acceptable salt thereof.

In some aspects, disclosed herein is a compound of Formula III:

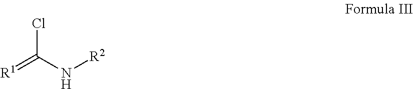

wherein: R¹ is selected from unsubstituted arylalkyl, substituted arylalkyl, or substituted alkyl; R² is selected from unsubstituted alkyl or substituted alkyl; or a pharmaceutically acceptable salt thereof.

In some embodiments, disclosed herein is a compound of Formula IIIa:

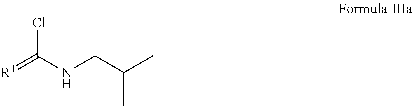

wherein: R¹ is selected from unsubstituted arylalkyl, substituted arylalkyl, or substituted alkyl; or a pharmaceutically acceptable salt thereof.

In some aspects, disclosed herein is a compound of Formula I:

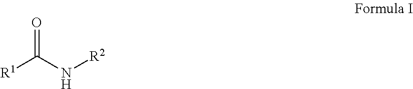

wherein: R¹ is selected from unsubstituted alkyl or substituted alkyl; R² is selected from substituted alkyl; or a pharmaceutically acceptable salt thereof.

In some embodiments, disclosed herein is a compound of Formula Ia:

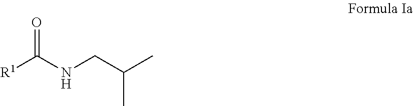

wherein: R¹ is selected from unsubstituted alkyl or substituted alkyl; or a pharmaceutically acceptable salt thereof.

In some aspects, disclosed herein is a compound of Formula II:

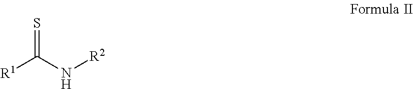

wherein: R¹ is selected from unsubstituted alkyl or substituted alkyl; R² is selected from substituted alkyl; or a pharmaceutically acceptable salt thereof.

In some aspects, disclosed herein is a compound of Formula IIa:

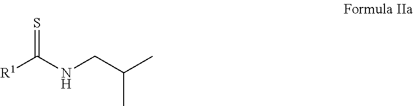

wherein: R¹ is selected from unsubstituted alkyl or substituted alkyl; or a pharmaceutically acceptable salt thereof.

In some aspects, disclosed herein is a compound of Formula III:

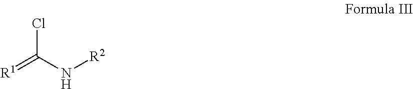

wherein: R¹ is selected from unsubstituted alkyl or substituted alkyl; R² is selected from substituted alkyl; or a pharmaceutically acceptable salt thereof.

In some embodiments, disclosed herein is a compound of Formula IIIa:

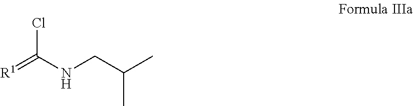

wherein: R¹ is selected from unsubstituted alkyl or substituted alkyl; or a pharmaceutically acceptable salt thereof.

In some embodiments, the compound of Formula I, II, or III is selected from the following compounds:

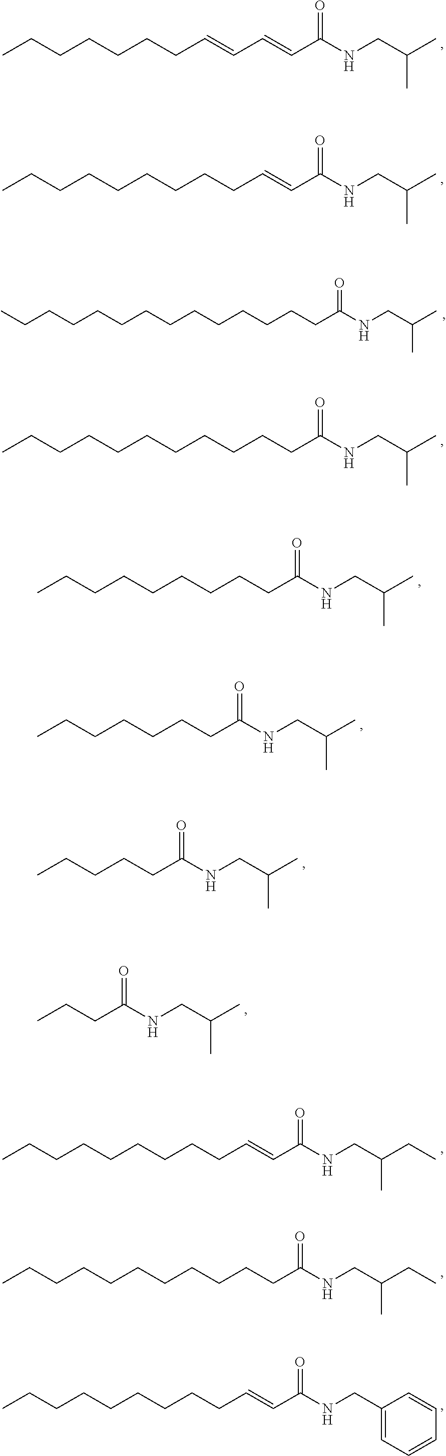

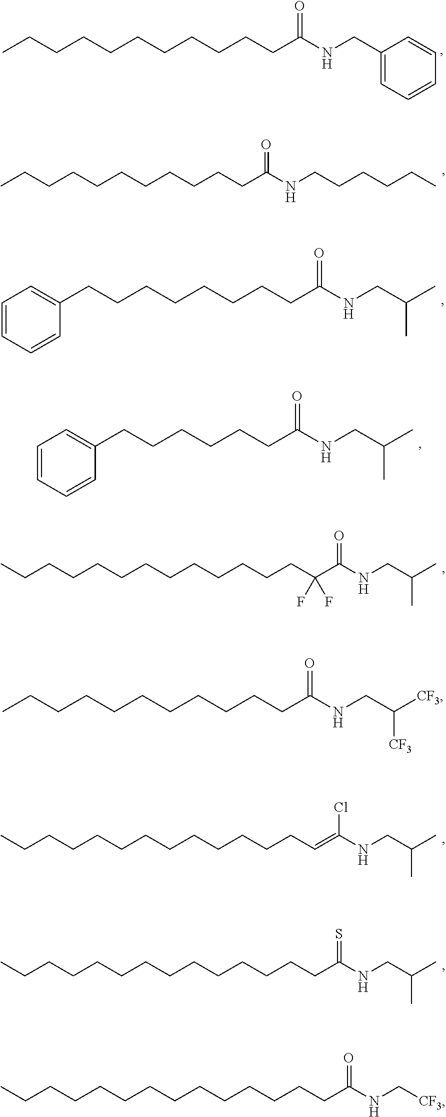

or a pharmaceutically acceptable salt thereof.

The syntheses of the compounds disclosed herein are further described in the examples below.

The inflammatory response is an important component of the normal immune response. Inflammation is an early step in the immune response, driven by macrophages in the tissues responding to an invading microbe. Macrophages produce soluble mediators that orchestrate the entry of additional immune cells into the infected tissue. The process is highly regulated and designed to stop quickly. Examples of acute dis-regulated inflammatory disease include sepsis, peritonitis, or meningitis. Examples of chronic dis-regulated inflammatory diseases would include rheumatoid arthritis, hepatitis, and pancreatitis. Inflammation is also triggered by injuries, wounds, and burns, and can be seen as a component of a huge list of diseases. NSAIDS, steroids and biologics are generally used to treat the chronic forms of these diseases.

However, allergic diseases occur through a different mechanism involving the mast cell and both chronic and acute forms are known. Examples of allergic diseases include, for example, hypersensitive reactions of the skin to metals, allergic responses to pollen (hay fever/allergic rhinitis) or pet dander, eczema, hives (urticaria), asthma, and food allergies. Allergic responses can be mild and annoying (hay fever, pet allergies) while other allergic responses can be rapidly fatal (peanut allergy). Anti-histamines, 5-LO inhibitors, and desensitization therapies are generally used to treat allergic disease. Steroids are also used to treat allergic reactions because of their non-specific suppressive effect and to treat the secondary inflammation. Other additional therapeutics for use in combination with an active compound disclosed herein can include corticosteroids, biologics, NSAIDs, or opiates.

In some embodiments, the compounds or compositions disclosed herein are used to treat or prevent an allergic disease. In some embodiments, the allergic disease is selected from a hypersensitive reaction of the skin to a metal, an allergic response to pollen, an allergic response to pet dander, an allergic response to house dust mites, an allergic response to molds, an allergic response to prescription medications, an allergic response to insect stings or bites, irritable bowel syndrome, eczema, urticaria, asthma, or a food allergy.

Also disclosed is a method of treating or preventing pain or itch, comprising administering to a subject in need thereof, a therapeutically effective amount of an active compound disclosed herein (Formula I, II, or III).

In some embodiments, disclosed herein are compositions comprising a compound of Formula I, and an additional therapeutic agent. In some embodiments, disclosed herein are compositions comprising a compound of Formula II, and an additional therapeutic agent. In some embodiments, disclosed herein are compositions comprising a compound of Formula III, and an additional therapeutic agent. In some embodiments, the additional therapeutic agent is selected from an anti-histamine (for example, diphenhydramine, chlorpheniramine, cetirizine, loratadine, fexofenadine, or desloratadine), a 5-LO inhibitor (for example, meclofenamate or zileuton), desensitization therapies, or a steroid (for example, beclomethasone, ciclesonide, mometasone, budesonide, triamcinolone, dexamethasone, hydrocortisone, prednisone, or deltasone).

In some embodiments, the application for the alkamides disclosed herein includes application to the skin to stop hypersensitive reactions or hives. In some embodiments, the alkamides are delivered to the intestine for treating or preventing food allergy. In other embodiments, alkamides are aerosolized for treating or preventing asthma or other respiratory allergic reactions.

In some embodiments, the application for the alkamides disclosed herein can include: a lotion or cream for the treatment of hypersensitive reactions of the skin accompanied by itch, pain, inflammation, and urticaria; a lotion or cream for the treatment of auto-inflammatory reactions of the skin such as eczema or psoriasis; aerosolized form for the treatment of inflammation and pain in the respiratory tract triggered by infection by respiratory viruses (colds and flu viruses); pill form for the treatment of gastrointestinal distress triggered by reactions to food and stress such as food allergies and irritable bowel syndrome; pill form for treatment of auto-inflammatory disease of the bowel such as colitis and Crohn's disease; injectable form for the treatment of acute and chronic inflammatory disorders such as sepsis, pancreatitis, peritonitis, and hepatitis; topical and injectable forms for the treatment of chronic pain such as diabetic neuropathy and back pain; or topical and injectable forms for the treatment of herpes virus induced pain and skin lesions.

### Compositions

Compositions, as described herein, comprising an active compound and an excipient of some sort may be useful in a variety of medical and non-medical applications. For example, pharmaceutical compositions comprising an active compound and an excipient may be useful for the treatment or prevention of an allergic disease.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semi-solid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacilic acid/acrylamide copolymer, polyalkylene oxides such as polyethylene oxide, polypropylene oxide, poly(ethylene oxide-propylene oxide), and a Pluronic polymer, polyoxyethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxyalkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

### Examples

The following examples are set forth below to illustrate the compounds, compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Mast Cell Degranulation and Calcium Influx are Inhibited by an Echinacea purpurea Extract and the Alkamide Dodeca-2E,4E-Dienoic Acid Isobutylamide

Native Americans used plants from the genus Echinacea to treat a variety of different inflammatory conditions including swollen gums, sore throats, skin inflammation, and gastrointestinal disorders. Echinacea spp. produce many fatty acid amides referred to as alkamides, which can inhibit cytokine, chemokine, and prostaglandin production from macrophages and T cells. Alkamides are thought to contribute to the anti-inflammatory activity of E. purpurea extracts by inhibiting production of inflammatory mediators.

In this example, the goal of this study was to evaluate the effects of the alkamide dodeca-2E,4E-dienoic acid isobutylamide (A15) on mast cells, which are important mediators of allergic and inflammatory responses, and to investigate the mechanism of alkamide inhibition of mast cell activation.

### Materials and Methods

A15 was evaluated for its effects on degranulation, calcium influx, cytokine and lipid mediator production using bone marrow derived mast cells (BMMCs) and the transformed rat basophilic leukemia mast cell line RBL-2H3. Methods included enzymatic assays, fluorimetry, ELISAs, and microscopy. A root extract of E. purpurea, and low and high alkamide-containing fractions prepared from this extract, were also tested for effects on mast cell function. Finally, A15 was tested for effects on calcium responses in RAW 264.7 macrophage and Jurkat T cell lines.

### Results

A15 inhibited the release of β-hexosaminidase and histamine from BMMCs and RBL-2H3 cells in a dose dependent manner. Inhibition occurred following stimulation with IgE anti-DNP/DNP-HSA or the calcium ionophore A23187. A15 prevented the rapid rise in intracellular calcium associated following FcεRI crosslinking and A23187 treatment showing it acts on the signals controlling granule release. An E. purpurea root extract and a fraction with high alkamide content derived from this extract also displayed these activities while fractions with little to no detectable amounts of alkamide did not. A15 mediated inhibition of calcium influx was not limited to mast cells as A23187-stimulated calcium influx was blocked in both RAW 264.7 and Jurkat cell lines. A15 also inhibited the release of TNF-α, and PGE₂ to a lesser degree, following A23187 stimulation indicating its broad activity on production of mast cell mediators.

In summary, these findings show that A15 and other alkamides are useful for treating allergic and inflammatory responses mediated by mast cells. More broadly, since calcium is a critical second messenger, the inhibitory effects of alkamides on calcium uptake would be predicted to dampen a variety of pathological responses, and may help explain the traditional uses of Echinacea.

Abbreviations A15, alkamide 15; β-hex, beta-hexosaminidase; BMMC, bone marrow-derived mast cell; CRAC, calcium-release activated calcium; CB₂, cannabinoid receptor type 2; ER, endoplasmic reticulum; FcεRI, fragment crystallizable epsilon receptor 1; IgE, immunoglobulin E; IP₃, inositol 1,4,5-trisphosphate; LPS, lipopolysaccharide; PLC-γ, phospholipase C-gamma; PGE₂, prostaglandin E₂; STIM-1, stromal interaction molecule 1; TNF-α, tumor necrosis factor alpha; Dodeca-2E,4E-dienoic acid isobutylamide (PubChem CID: 6443006)

### 1. Introduction

Allergic diseases impact millions of people and the prevalence of these disorders continues to rise in the United States and other developed countries (Pawankar et al., 2013). Allergic disease can affect a variety of organs and tissues including the skin (hypersensitive reactions), lungs and respiratory tract (asthma and rhinitis), and gastrointestinal tract (food allergies and irritable bowel syndrome). Mast cells, found in mucosal and serosal tissues, contain large, dense granules filled with pre-formed mediators and are critical to allergic responses (reviewed by (Wernersson and Pejler, 2014)). Basophils are also important to allergic disease and share many features with mast cells including mediator-containing granules and high affinity IgE receptors (FcεRI). Within seconds after activation, mast cells and basophils exocytose their granules releasing histamine, cytokines/chemokines, proteases, and growth factors into the surrounding tissue causing the symptoms of allergic disease. In addition to the release from granules, mast cells and basophils can also produce cytokines, chemokines, prostaglandins, and leukotrienes by de novo biosynthetic pathways (Vig et al., 2008). Therapeutics that block granule release, or stabilize mast cells, have been intensely sought after as treatments for allergic disease (Finn and Walsh, 2013).

Mast cells are activated when IgE antibodies bound to FcεRI are crosslinked by multivalent antigens and initiate intracellular signaling (Rivera et al., 2008). Mast cell activation is a calcium-dependent process. Receptor crosslinking activates phospholipase C-γ (PLC-γ), leading to production of inositol 1,4,5-trisphophate (IP₃), which in turn causes the release of calcium from the endoplasmic reticulum (ER). Depletion of calcium in the ER leads to activation of an ER membrane-associated calcium sensor, stromal interaction molecule 1 (STIM-1) (Baba et al., 2008; Hoth and Penner, 1992). STIM-1 translocates to the plasma membrane to interact with Orail, the pore-forming subunit of the calcium release-activated calcium (CRAC) channel. STIM-1 oligomerizes, causing the CRAC channel to form and extracellular calcium to enter the cytosol (Baba et al., 2006; Luik et al., 2006). Mast cells can also be activated by treatment with calcium ionophores such as A23187, which bypass FcεRI-mediated signaling events and directly cause increased levels of cytosolic calcium by transporting extracellular calcium across the plasma membrane and by emptying ER calcium stores into the cytosol (Dedkova et al., 2000).

Native Americans used extracts from Echinacea spp. (coneflowers) to treat a variety of conditions associated with inflammatory and allergic disease, including; swollen gums, inflamed skin, sore throats, and intestinal pain (reviewed by (Kindscher, 1989)). Today, Echinacea extracts are used primarily to treat upper respiratory infections (reviewed by (Woelkart et al., 2008)). In vitro, a number of studies have confirmed that Echinacea extracts can suppress production of inflammatory mediators from macrophages and T lymphocytes (Cech et al., 2010; LaLone et al., 2007; Spelman et al., 2009) Recently, several reports have confirmed in vivo that Echinacea extracts also suppress allergic responses. For example, an Echinacea extract reduced several parameters of allergy in ovalbumin-sensitized guinea pigs (Sutovska et al., 2015). Similarly, an Echinacea extract was shown to modulate disease in a model of atopic eczema (Olah et al., 2017).

Alkamides, also known as alkamides, are a class of fatty-acid-like molecules whose activity has been linked to the anti-inflammatory activity of Echinacea extracts (Cech; et al., 2010; LaLone et al., 2007; Spelman et al., 2009). Alkamides are produced by a number of medicinal plants including Echinacea spp., Acmella oleracea, and Zanthoxylum americanum (reviewed by (Boonen et al., 2012)). Each alkamide contains an amine group, an alkyl chain, and a functional group such as isobutyl, benzyl, or methyl group. The number of carbons in the alkyl chain can vary, as well as the number and position of double and triple bonds. Alkamide structures can be further diversified with modifications including hydroxylations and methylations (Leyte-Lugo et al., 2015). Certain plant species can produce multiple alkamides. For example, over 18 different alkamides have been identified from E. purpurea (Bauer et al., 1988; Hou et al., 2010; Leyte-Lugo et al., 2015). The alkamide dodeca-2E,4E-dienoic acid isobutylamide, whose activity was examined in this report, is referred to as alkamide 15 (A15) according to a numbering system reported previously (Cech et al., 2006). Alkamides are capable of suppressing cytokine, chemokine, and prostaglandin production from a number of immune cell types including monocytes, macrophages, and T cells (Cech; et al., 2010; LaLone et al., 2007; Raduner et al., 2006; Spelman et al., 2009). This activity has led a number of investigators to suggest that alkamides contribute to the anti-inflammatory, medicinal activity of these plants (Sharma et al., 2009; Todd et al., 2015; Woelkart and Bauer, 2007).

Several groups have investigated the mechanism of cytokine suppression by alkamides. For example, the alkamides dodeca-2E,4E,8Z,10Z(E)-tetraenoic acid isobutylamide (A11a/b) and A15 were shown to bind the cannabinoid type 2 receptor (CB₂) and proposed to enhance constitutive IL-6 production from human whole blood by signaling through CB₂ (Raduner et al., 2006). However, these authors also reported that alkamide inhibition of TNF-α, IL-1β, and IL-12p70 from LPS-stimulated human whole blood was mediated through CB₂-independent effects. In another report, the alkamide A11a/b reduced TNF-α production from LPS-stimulated RAW 264.7 cells, which was attributed to c-Jun N-terminal kinases (JNK)-mediated upregulation of heme oxygenase-1 (Hou et al., 2011).

### 2. Materials and Methods

### 2.1 Reagents

All chemicals were purchased from either Sigma-Aldrich (St. Louis, Mo.) or Thermo Fisher Scientific (Waltham, Mass.). Dodeca-2E,4E-dienoic acid isobutylamide (A15) was synthesized at North Carolina State University (Raleigh, N.C.) as described previously (Moazami et al., 2015). In brief, a two-step oxidation of the commercially available diene-containing alcohol was performed to create the carboxylic acid followed by coupling with isobutyl amine (T3P®). This process provided A15 in good yield and proved identical to the natural product by ¹H and ¹³C NMR analysis.

### 2.2 Mice

Male and female C57BL/6 mice (The Jackson Laboratory, Bar Harbor, Me.) were housed at the Biological Resources Facility at North Carolina State University. All experiments were approved by the IACUC at North Carolina State University.

### 2.3 Cell Isolation and Culture

Bone marrow cells were isolated from the femurs of 6-8 week old C57BL/6 mice and cultured in RPMI-1640 containing 10% heat-inactivated FBS, non-essential amino acids, HEPES buffer, sodium pyruvate, 100 U/mL penicillin, 100 μg/mL streptomycin, 5 ng/mL mIL-3, and 5 ng/mL mSCF for 4-6 weeks until mature, bone marrow-derived mast cells (BMMCs) were obtained, as described previously (Kuehn et al., 2010). Differentiation into >98% mast cells was confirmed through toluidine blue staining (1%, pH 1). RBL-2H3, RAW 264.7, and Jurkat cells were obtained from the American Type Culture Collection (Manassas, Va.). RBL-2H3 cells were cultured in MEM supplemented with 15% heat inactivated FBS, non-essential amino acids (Sigma-Aldrich), and sodium pyruvate (Sigma-Aldrich). RAW 264.7 and Jurkat cells were cultured in DMEM supplemented with 10% FBS. FBS was obtained from Gemini Bio-Products (Sacramento, Calif.). For TNF-α, prostaglandin, and histamine measurements, cells were stimulated with A23187 alone or in combination with A15 for 8 hrs or 1 hr for histamine release. Supernatants were collected, centrifuged at 16,000×g for 5 min and stored at −80° C. until analysis. TNF-α sandwich ELISA kits, or PGE₂ and histamine competitive direct EIA kits were purchased from eBioscience (San Diego, Calif.), Enzo Life Sciences (Farmingdale, N.Y.), or Oxford Biomedical Research (Rochester Hills, Mich.), respectively. Optical density was determined using a Synergy HT microplate reader (BioTek Instruments, Inc, Winooski, Vt.). Concentrations of each analyte were interpolated from standard curves.

### 2.4 β-Hexosaminidase Degranulation Assay

RBL-2H3 cell and BMMC degranulation was measured using a β-hexosaminidase (β-hex) activity assay. RBL-2H3 cells were plated in 96-well flat-bottom plates and BMMCs were plated in 96-well v-bottom plates to allow cells to pellet during centrifugation. Cells were incubated for 4 hr before media was aspirated and replaced with vehicle or media containing 1 μg/mL mouse IgE anti-DNP (SPE-7) (Sigma-Aldrich) and incubated overnight. Prior to stimulation, cells were rinsed with calcium-containing Tyrode's buffer (1.8 mM CaCl₂, 135 mM NaCl, 5 mM KCl, 5.6 mM glucose, 1 mM MgCl₂, 0.1% BSA, 20 mM HEPES, pH 7.4). All treatments were prepared in Tyrode's buffer. RBL-2H3 cells were stimulated with either A23187 (Sigma-Aldrich) or DNP-HSA (Sigma-Aldrich) alone or in combination with A15 and incubated for 1 hr at 37° C. Ethanol was used as the vehicle for A15 and the final concentration was 1% in all degranulation experiments. Wells were then aspirated, and solutions containing A23187 alone or in combination with A15 were added and incubated for 1 hr at 37° C. After the incubation, 30 μL of supernatant was collected and incubated with 10 μL of 3.4 mg/mL p-nitrophenyl N-acetyl-β-D-glucosaminide (p-NAG) at 37° C. for 1 hr to measure the amount of β-hex released from the cells. Excess supernatant was removed and the remaining cells were lysed with 100 μL of 0.1% Triton™ X-100 in Tyrode's buffer. 30 μL of the cell lysate was incubated with 10 μL of p-NAG to determine the amount of β-hex remaining in the cells. The reaction was stopped by adding 100 μL of 0.2 M sodium carbonate buffer. Absorbance was read at 405 nm on a BioTek Synergy HT microplate reader. The percent degranulation was calculated as the A₄₀₅ of the supernatant divided by total absorbance (A₄₀₅ of the supernatant+A₄₀₅ of the lysate)×100.

### 2.5 LDH Cytotoxicity Assay

RBL-2H3 cells were seeded into 96-well plates and incubated overnight prior to treatment with a range of A15 concentrations or 10 μg/mL cycloheximide. Supernatants were collected after 18 hr and analyzed for lactate dehydrogenase (LDH) activity using the commercially available Pierce LDH Cytotoxicity Assay Kit according to the manufacturer's instructions (Thermo Fisher Scientific). Briefly, in a 96-well plate, 50 μL of supernatant were incubated with 50 μL of reaction mixture containing a tetrazolium salt that is reduced to formazan in the presence LDH for 30 min protected from light. 50 μL of stop solution was added, and the absorbance was read at 490 nm with absorbance at 690 nm subtracted to remove background absorbance. LDH release from vehicle treated cells was used to determine spontaneous LDH release. Additionally, lysis buffer was used to determine maximum LDH release.

### 2.6 Calcium Assay

RBL-2H3, BMMC, RAW 264.7, or Jurkat cells were plated in clear-bottom, black, 96-well plates (BioExpress, Kaysville, Utah) and incubated overnight with media only or media containing IgE anti-DNP. Cells were loaded with the calcium-sensitive dye fluo-4 AM using the Fluo-4 Direct™ Calcium Assay Kit (Thermo Fisher Scientific) according to the manufacturer's instructions by incubating at 37° C. for 30 min followed by 30 min at room temperature. Prior to stimulation, baseline fluorescent readings were measured from triplicate wells in 5 s intervals for 1 min using a Synergy HT microplate reader with 485/20 nm excitation and 528/20 nm emission filters. Cells were then treated with either 1 μM A23187 or 50 ng/mL DNP-BSA prepared in calcium- and magnesium-containing or calcium- and magnesium-free HBSS with 20 mM HEPES, and fluorescence was measured in 10 s intervals for 2 min. The final concentration of ethanol was 0.25% in these assays. The average baseline fluorescence of each well was subtracted from the stimulated fluorescent values to calculate the change (Δ) in RFU's and graphed as shown.

### 2.7 Fluorescence Microscopy

Cells loaded with fluo-4 were treated and observed with an Axioskop 2 microscope (Zeiss, Oberkochen, Germany) using a fluorescent illuminator HXP-120 light source and images were acquired at 30, 60, or 120 sec after stimulation with a AxioCam MRc5 camera (Zeiss) and AxioVision software (Zeiss).

2.8 Separation and Fractionation of an Echinacea purpurea Extract

Echinacea purpurea (L) Moench roots were purchased from Pacific Botanicals (Grants Pass, Oreg.). A voucher specimen (NCU 633811) was deposited at the North Carolina Herbarium. An ethanolic extract (EE) was prepared and fractionated as described previously (Todd et al., 2015). Briefly, roots were collected, dried, and ground mechanically prior to extraction in 75% ethanol for seven days. A liquid-liquid partitioning scheme was used to generate an alkamide containing chloroform layer (CL), which was subsequently fractionated using normal-phase flash chromatography over a RediSep Rf silica gel column. The eluent was collected in 13 different fractions (F1-13). Concentrations of the most abundant alkamides were quantified in each sample using LC-MS and used to estimate the total alkamide content (μg/mg extract). EE=51±8.2, CL 140±4.7, F2=below limit of quantification, F6=310±42, F8=0.14±0.034 (Todd et al., 2015).

### 2.9 Statistical Analysis

Significant differences between means were determined using, a Student's unpaired t-test, a one-way ANOVA, or a repeated measures one-way ANOVA with GraphPad Prism version 5.0 software (GraphPad Software, La Jolla, Calif.). Tests used and levels of significance are indicated in individual figure legends.

### 3. Results

### 3.1 A15 Inhibits Mast Degranulation

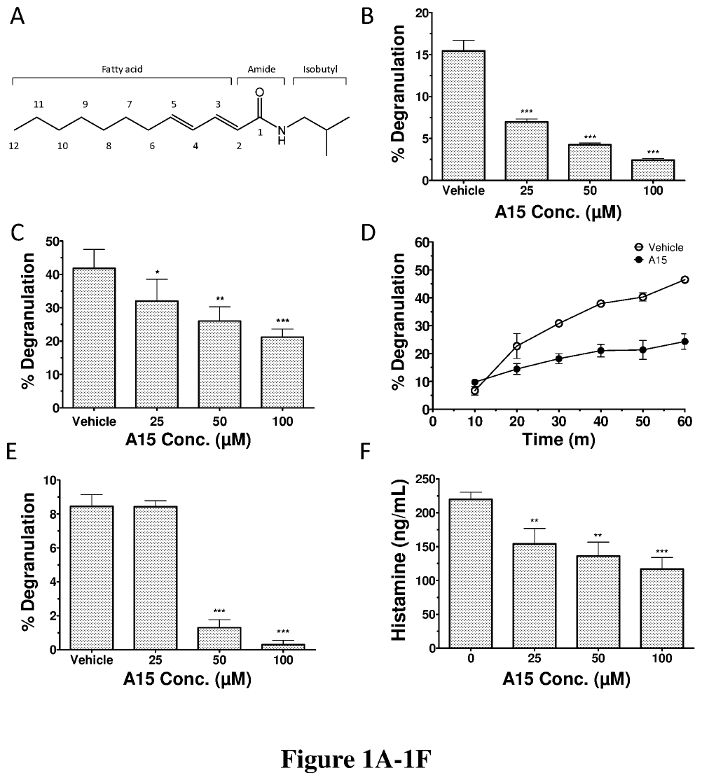

In these experiments, two populations of mast cells were examined; primary bone marrow derived mast cells (BMMCs) from C57BL/6 mice, and the cell line RBL-2H3, a rat basophil-derived, transformed cell line whose degranulation pathways are similar to those of primary mast cells and basophils and frequently used to study mast cell biochemistry (reviewed by (Passante and Frankish, 2009)). With both cell types, degranulation was induced by treatment with either IgE anti-DNP and DNP-HSA or the calcium ionophore A23187. To monitor degranulation, the release of two granule components, the enzyme β-hexosaminidase (β-hex) and the vasoactive amine histamine, was measured. The alkamide used in these studies was A15 because in experiments with monocytes and macrophages it displayed potent and broad cytokine inhibitory activity (Cech; et al., 2010; Raduner et al., 2006). Also, A15 can be synthesized from simple starting materials (Moazami et al., 2015). As shown in FIG. 1A, A15 contains an isobutyl head group, an amide group, and a 12-carbon fatty acid chain with double bonds positioned at carbons 2 and 4.

FIG. 1 panels B-F, summarize key findings for the effects of A15 on mast cell degranulation. As shown in FIG. 1B, A15 inhibited the release of β-hex from A23187-stimulated BMMCs in a dose dependent fashion with a maximum inhibition of 83.5%±2.8% at a concentration of 100 μM. A15 also inhibited the release of β-hex from A23187-stimulated RBL-2H3 cells although the level of inhibition was less than with BMMCs (48.4%±3.7% at 100 μM) (FIG. 1C). As shown in FIG. 1D, in experiments with A23187-treated RBL 2H3 cells, inhibition of β-hex release was apparent within 20 min after treatment began. FIG. 1E shows that A15 also inhibited degranulation from RBL-2H3 cells when they are activated by IgE anti-DNP and DNP-BSA. Finally, in FIG. 1F, addition to β-hex, A15 also inhibited the release of the vasoactive amine histamine from RBL-2H3 cells treated with A23187.

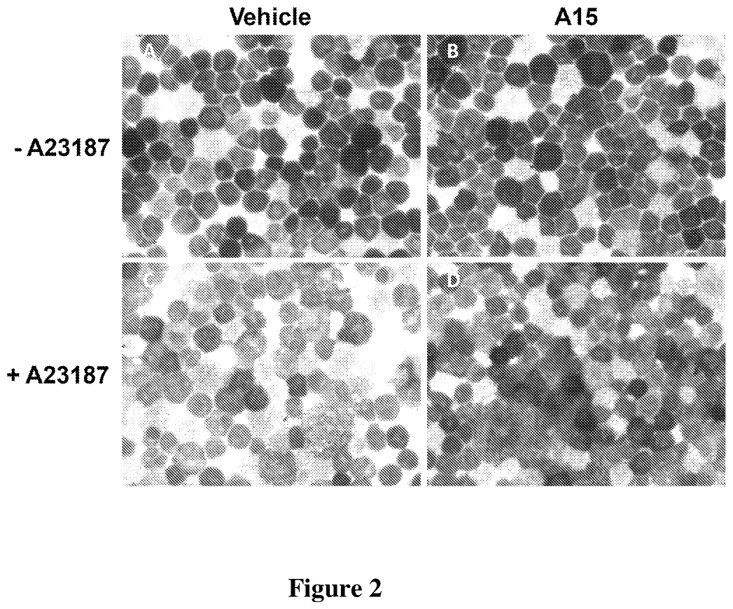

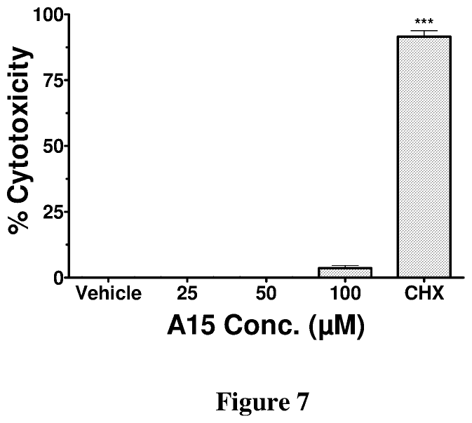

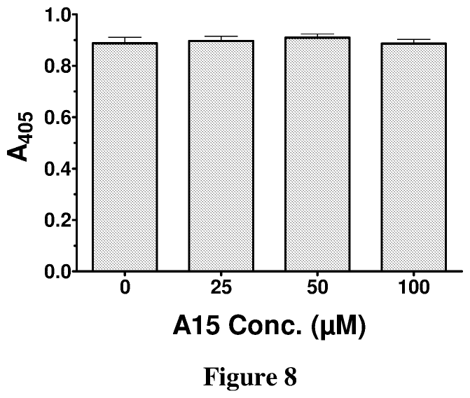

The data presented in FIG. 1 shows that A15 acts biochemically to block degranulation from BMMCs and RBL-2H3 cells; however, because alkamides have not been tested with these cells previously, it was sought to reveal other explanations for these results. Lactate dehydrogenase release assays were performed to ensure that the results reported in FIG. 1 did not arise from a cytotoxic effect. As shown in FIG. 7, there was no significant cytotoxic activity of A15 towards RBL-2H3 cells, even after overnight treatment, in agreement with previous studies of A15 and RAW 264.7 macrophages (Moazami et al., 2015). In addition, it was tested whether A15 could inhibit the enzymatic activity of β-hex itself. As shown in FIG. 8, when RBL-2H3 cell lysates were incubated with the substrate (p-NAG) alone or in the presence of the indicated concentrations of A15, production of the chromogenic product was not inhibited. Finally, as shown in FIG. 2, the inhibitory effects of A15 on degranulation were apparent after staining with toluidine blue. Toluidine blue is a metachromatic dye which stains nuclei blue and mast cell granules purple. Treatment of BMMCs with A23187 for 30 min noticeably reduced staining compared to control cells (FIGS. 2A and C), which was blocked by A15 (FIG. 2D) confirming that A15 does indeed block release of granules from BMMCs. Treatment with A15 itself did not noticeably affect staining (FIG. 2B).

### 3.2 A15 Blocks Calcium Influx in BMMCs and RBL-2H3 Cells

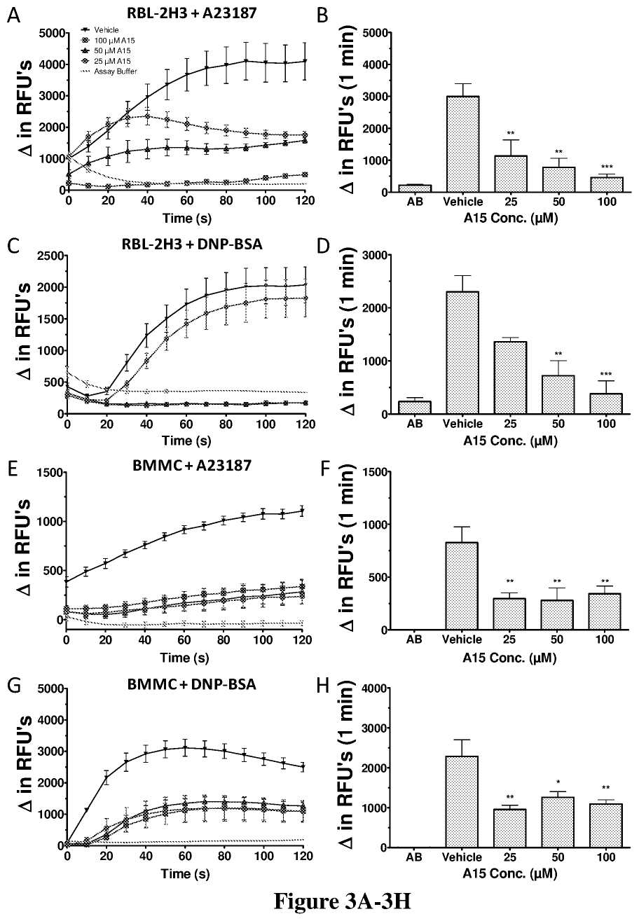

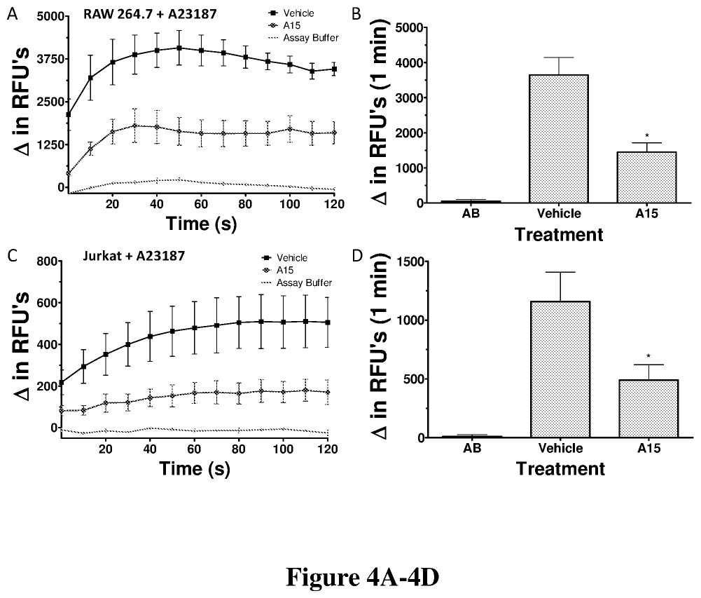

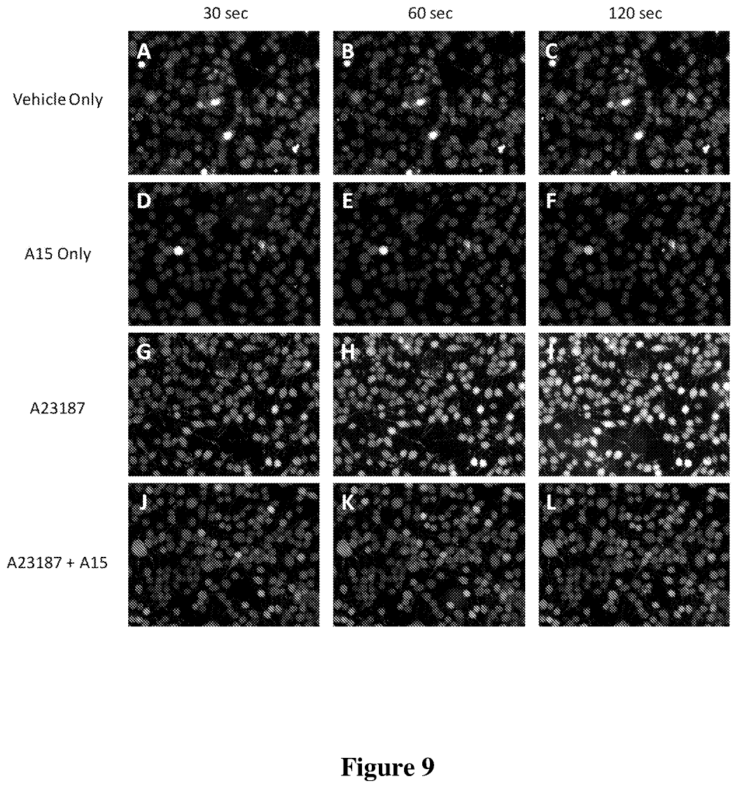

Since A15 blocked both FcεRI and A23187-mediated degranulation, its effects likely occur via an element common to both pathways. Calcium is a key second messenger in the activation pathways of mast cells and basophils (reviewed by (Gilfillan and Tkaczyk, 2006)). Therefore, it was hypothesized that A15 was acting to inhibit calcium mobilization in BMMCs and RBL-2H3 cells. To address this hypothesis, the calcium-sensitive fluorescent dye fluo-4 AM was utilized. Cells were loaded with the dye, stimulated, and a multichannel absorbance plate reader was used to quantitatively examine levels of intracellular calcium. As shown in FIG. 3A, a rapid rise in levels of intracellular calcium in RBL-2H3 cells treated with A23187 was observed. The increase was noticeable after −10 sec and tended to plateau after ˜60-80 sec. where it was found that treatment with A15 blocked this response in a dose-dependent fashion (FIG. 3A) with 83.2%±5.5% inhibition measured at the 100 μM concentration of A15 (FIG. 3B). A15 also blocked the calcium response in RBL-2H3 cells treated with IgE anti-DNP and DNP-BSA (FIGS. 3C and D) and in BMMCs treated with either A23187 (FIGS. 3E and F) or IgE anti-DNP and DNP-BSA (FIGS. 4G and H). Again, readily visible by microscopy, treatment of RBL-2H3 cells with A23187 induced an increase in fluorescence, which was blocked by A15 (FIG. 9). It should also be noted that A15 did not cause a significant change in the level of constitutive fluorescence, compared to vehicle, in unstimulated cells. For example, in FIG. 9, analysis of pixel intensity did not reveal a significant change in fluorescence with A15 treatment (p>0.05, n=5 fields) compared with a significant 158.4% increase with A23187 treatment (p<0.0001, n=5 fields).

### 3.3 A15 Inhibits Calcium Influx in A23187-Stimulated RAW 264.7 and Jurkat Cells

Calcium influx occurs in many cell types other than mast cells via similar store-operated calcium entry-dependent mechanisms (reviewed by (Feske, 2009; Vig and Kinet, 2009)). To determine if the suppression by A15 was specific to mast cells or occurred with other cell types, fluo-4 AM-loaded RAW 264.7 macrophage-like cells (FIG. 4A) or Jurkat T cells (FIG. 4B) cells were stimulated with 1 μM A23187 and/or 100 μM A15. A15 treatment significantly reduced calcium influx in both RAW 264.7 and Jurkat cells at 1 min with 60.2% and 43.6% suppression, respectively, suggesting that A15 is a general inhibitor of calcium influx.

3.4 an Ethanolic E. purpurea Root Extract and High-Alkamide Fractions Display Inhibitory Activities

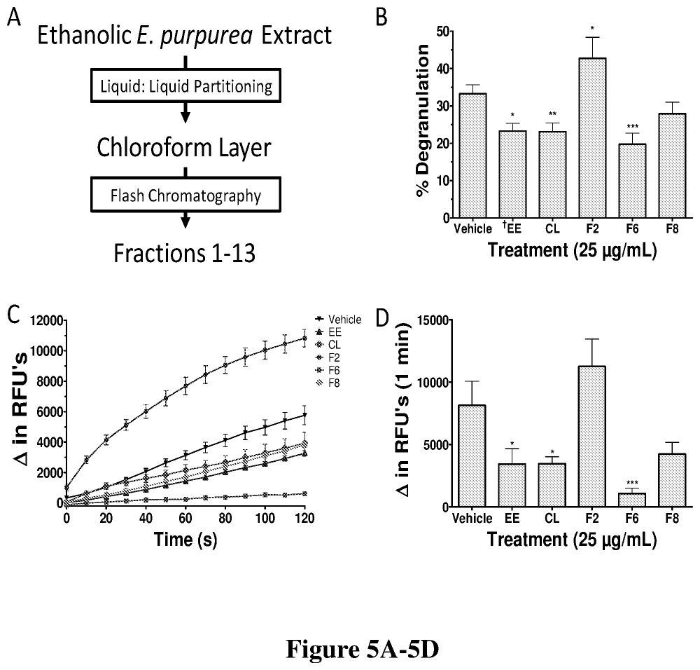

Since E. purpurea is a natural source for alkamides including A15, it was hypothesized that an E. purpurea extract would display mast cell inhibitory activity. To test this hypothesis, an extract and fractions separated from that extract was examined, which were characterized previously for their effects on macrophage TNF-α production (Todd et al., 2015). The extract was prepared originally from E. purpurea roots macerated in 75% ethanol. Liquid:liquid partitioning was then used to purify a high alkamide chloroform layer that was then separated into fractions with varying levels of alkamides by flash chromatography over a C18 column (FIG. 5A). A total of 18 fractions were collected, 3 of which (6, 7 and 8) contained detectable levels of alkamides. The extract and fractions were then characterized for effects on LPS-stimulated production of TNF-α by RAW 264.7 macrophage-like cells (Todd et al., 2015).

For these experiments, the extract and a subset of fractions that contained distinct alkamide content was tested and displayed distinct activities toward TNF-α production. As shown in FIGS. 5B and C, the extract (EE, 51±8.2 μg/mg) did indeed inhibit both degranulation and calcium movement with A23187-treated RBL-2H3 cells. The chloroform layer (CL, 140±4.7 μg/mg) also displayed similar inhibitory activities (FIGS. 5B and C). Fraction 6 (also with high alkamide content, i.e. 310±42 μg/mg extract) was the only fraction tested that produced statistically significant inhibition of degranulation or calcium influx. Fraction 8, with low alkamide content, (0.14±0.034 μg/mg extract) did not produce significant inhibition of degranulation or calcium influx (FIG. 5B). Together these results show that inhibition of degranulation and calcium influx in mast cells requires alkamides, and that other compounds can impact TNF-α production by macrophages. It should be noted that a suppressive trend was noted for calcium influx with fraction 8, showing that the low level of alkamides, or perhaps other molecules, are able to weakly reduce calcium influx (FIGS. 5C and D). Also, fraction 2 enhanced degranulation and calcium influx (FIG. 5B-D). Fraction 2 lacks detectable levels of alkamides and was included as a control since it was previously found to stimulate cytokine and chemokine production from RAW 264.7 macrophages and contained LPS and potentially other pathogen-associated molecular patterns (Todd et al., 2015). These compounds appear to exert similar effects on mast cells.

### 3.5 A15 Inhibits De Novo Production of PGE₂ and TNF-α in RBL-2H3 Cells

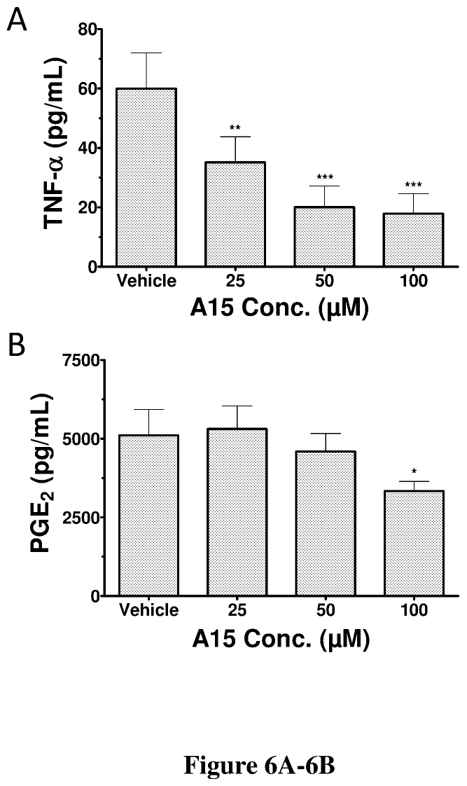

In addition to the rapid degranulatory process, mast cells initiate synthesis of newly formed pro-inflammatory mediators, including TNF-α and PGE₂, which contribute to the long-term inflammatory responses associate with allergy (Vig et al., 2008). To test if A15 blocked these responses, RBL-2H3 cells were treated with A23187 and/or A15, overnight supernatants collected, and analyte concentration determined by ELISA. As shown in FIG. 6B, A15 produced significant, dose dependent inhibition of TNF-α at all doses tested. In contrast, production of PGE₂ was weakly inhibited and only at the 100 μM concentration of A15.

### 4. Discussion

Alkamides have been linked to the therapeutic effects of a number of medicinal plants (reviewed by (Boonen et al., 2012)). Although alkamide suppression of cytokine production has been described previously, the mechanism by which alkamide-containing medicinal plants mediate their effects remains controversial (Boonen et al., 2012; Greger, 2016). In this report, the effects of the alkamide A15 on mast cell degranulation and calcium influx were evaluated and found that it inhibited degranulation from RBL-2H3 cells and BMMCs (FIGS. 1 and 2), and this effect correlated with the suppression of calcium influx (FIG. 3). The effects of A15 on degranulation and calcium movement were not attributed to cytotoxicity (FIG. 7). Additionally, inhibition of calcium influx was not specific to mast cells as A15 also inhibited A23187-stimulated calcium influx in RAW 264.7 macrophages and Jurkat T cells (FIG. 4). Inhibition of mast cell degranulation and calcium influx was observed by E. purpurea extracts and fractions with high alkamide content, but not by fractions with little to no detectable alkamide levels (FIG. 5). Lastly, A15 inhibited de novo production of TNF-α and PGE₂ (FIG. 6).

In mast cells and basophils, crosslinking of the FcεRI leads to activation of PLC-γ, whose activity ultimately leads to release of calcium from ER stores followed by the influx of extracellular calcium through the CRAC channel. In contrast, ionophore treatment can transport calcium ions directly across the plasma membrane to increase calcium levels and can also cause rapid depletion of ER calcium stores and subsequent CRAC channel formation in the plasma membrane, bypassing upstream FcεRI- and PLC-γ-mediated events. A possible explanation for the effect of A15 is that it could chelate calcium ions to prevent influx, which would block both FcεRI- and ionophore-mediated calcium influx. However, alkamides have not been reported to bind divalent ions previously, and known calcium chelators, such as EGTA, contain a cavity with multiple carboxyl groups to bind a calcium ion, which A15 lacks (Tsien, 1980). More likely, A15 exerts direct or indirect effects on the CRAC channel. For example, A15 could bind directly to the CRAC channel, perhaps inserting into the pore to block influx of extracellular calcium. Or, A15 could inhibit the association of STIM1 with Orail thereby preventing channel formation and influx of extracellular calcium. Translocation of STIM-1 to the plasma membrane occurs by movement along microtubules, and therefore, if A15 disrupts microtubule assembly, this could also explain the inhibition of calcium influx through the CRAC channel (Smyth et al., 2007). Alternatively, A15 could have a disruptive effect on membranes causing membrane-spanning ion channels (in the plasma membrane or the ER) to change conformation and prevent ion movement. Several of these hypotheses could also explain the ability of other alkamides to inhibit the movement of potassium and sodium ions in neurons (Bautista et al., 2008; Ottea et al., 1990; Tsunozaki et al., 2013).

In these experiments, A15 substantially blocked production of TNF-α□ from RBL-2H3 cells, but its inhibitory effect on PGE₂ production was less, similar to results found previously with RAW 264.7 macrophage-like cells (Cech; et al., 2010). Production of both TNF-α and PGE₂ both likely involve calcium-dependent processes (Gronich et al., 1990; Vig et al., 2008). In mast cell types, for example, TNF-α can be released preformed from granules, which is calcium-dependent, and synthesized through a de novo, transcription-dependent pathway which can involve calcium-dependent transcription factors such as nuclear factor of activated T cells (NFAT) (Klein et al., 2006). The PGE₂ pathway may also involve calcium-dependent transcription factors and calcium is also required for activation of cytosolic phospholipase A₂, the first enzyme in the biosynthetic pathway which cleaves arachidonic acid from membrane phospholipid (Gronich et al., 1990). At present, it is unclear why PGE₂ production is relatively resistant to treatment with A15. Calcium responses may recover after several hours of treatment with A15, which are sufficient to drive production of PGE₂ in overnight assays. In contrast, the production of TNF-α may be more highly dependent on the immediate calcium response which was strongly inhibited by A15.

Alkamides have been linked to the anti-inflammatory effects of E. purpurea and shown to inhibit cytokine, chemokine and prostaglandin production from influenza A-stimulated macrophages in vitro (Cech; et al., 2010; LaLone et al., 2007). Although the role of alveolar macrophages in response to influenza infection is well-known, it was recently reported that mast cells are critical to the pathogenesis of influenza infection (Graham et al., 2013). These findings suggest that the beneficial effects of E. purpurea extracts for colds and flu could be due to inhibition of mast cell degranulation, a possibility that has not been explored previously. It is also possible that E. purpurea extracts with high alkamide content could be useful for treating other forms of allergic inflammation mediated by mast cells such as hypersensitive reactions of the skin (Olah et al., 2017), asthma (Sutovska et al., 2015), or allergic gastrointestinal disorders (in accord with the original use of the plant by Native Americans).

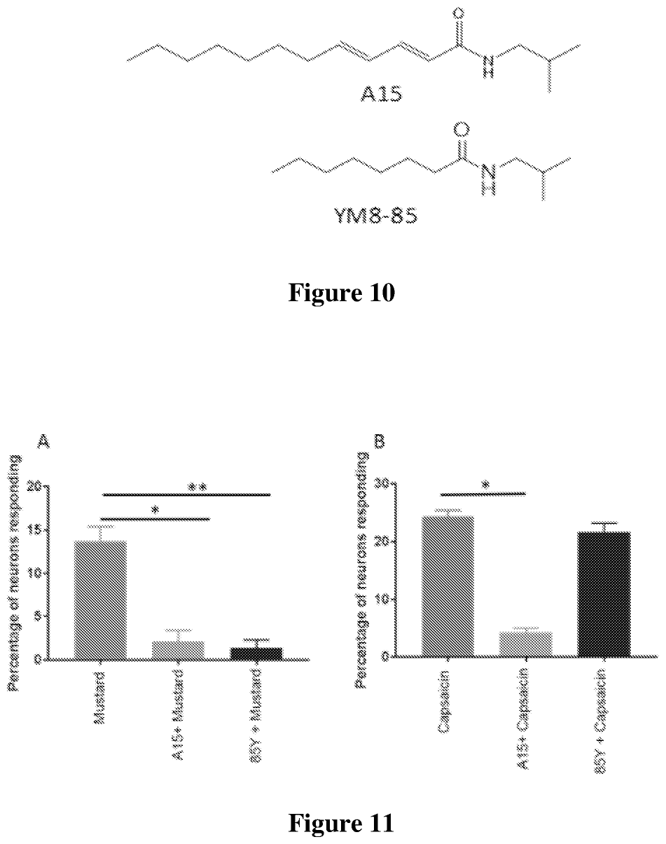

In addition to their effects on cytokine and chemokine production, alkamides, including spilanthol and sanshool, have been linked to the tingling, analgesic activity of extracts made from alkamide-producing plants such as E. purpurea, A. oleracea and Z americanum (Albin and Simons, 2010; Bryant and Mezine, 1999; Ley et al., 2006). The numbing effects of alkamides have been linked to activation of the capsaicin receptor, TRPV1 (Sugai et al., 2005). In addition, alkamides have been shown to inhibit potassium movement through two-pore domain potassium channels (KCNK3, KCNK9, and KCNK18), which are anesthetic-targeted channels (Bautista et al., 2008; Liu et al., 2004; Talley and Bayliss, 2002). Interestingly, Bautista et al. found that the treatment of neurons with 100 μM sanshool caused calcium influx that was dependent on extracellular calcium (Bautista et al., 2008). In contrast, with RBL-2H3 cells or BMMCs, A15 itself did not cause a calcium response (FIG. 10D-F) perhaps suggesting differences in the activity of A15 and sanshool, or varying effects of alkamides on neurons versus mast cells.

In conclusion, the alkamide A15 from E. purpurea inhibits mast cell degranulation and calcium influx. The beneficial effects of E. purpurea extracts to patients with upper respiratory tract infections may be due to the suppressive effects of alkamides on mast cell activation. It remains to be determined if structural improvements to A15 could result in a molecule with increased activity. The combined effects of A15 on mast cell activation with previous reports of cytokine suppression from macrophages supports the use of A15 and E. purpurea extracts with high alkamide content for limiting inflammation associated with infections as well as allergic responses.

### REFERENCES CITED IN THIS EXAMPLE

Albin, K. C., Simons, C. T., 2010. Psychophysical Evaluation of a Sanshool Derivative (Alkamide) and the Elucidation of Mechanisms Subserving Tingle. PLoS ONE 5(3), e9520.
Baba, Y., Hayashi, K., Fujii, Y., Mizushima, A., Watarai, H., Wakamori, M., Numaga, T., Mori, Y., lino, M., Hikida, M., 2006. Coupling of STIM1 to store-operated Ca2+ entry through its constitutive and inducible movement in the endoplasmic reticulum. Proceedings of the National Academy of Sciences 103(45), 16704-16709.
Baba, Y., Nishida, K., Fujii, Y., Hirano, T., Hikida, M., Kurosaki, T., 2008. Essential function for the calcium sensor STIM1 in mast cell activation and anaphylactic responses. Nat Immunol 9(1), 81-88.
Bauer, R., Remiger, P., Wagner, H., 1988. Alkamides from the roots of Echinacea purpurea. Phytochemistry 27(7), 2339-2342.
Bautista, D. M., Sigal, Y. M., Milstein, A. D., Garrison, J. L., Zorn, J. A., Tsuruda, P. R., Nicoll, R. A., Julius, D., 2008. Pungent agents from Szechuan peppers excite sensory neurons by inhibiting two-pore potassium channels. Nature Neuroscience 11(7), 772-779.
Boonen, J., Bronselaer, A., Nielandt, J., Veryser, L., De Tré, G., De Spiegeleer, B., 2012. Alkamid database: Chemistry, occurrence and functionality of plant N-alkamides. Journal of Ethnopharmacology 142(3), 563-590.
Bryant, B. P., Mezine, I., 1999. Alkamides that produce tingling paresthesia activate tactile and thermal trigeminal neurons. Brain Research 842(2), 452-460.
Cech, N. B., Eleazer, M. S., Shoffner, L. T., Crosswhite, M. R., Davis, A. C., Mortenson, A. M., 2006. High performance liquid chromatography/electrospray ionization mass spectrometry for simultaneous analysis of alkamides and caffeic acid derivatives from Echinacea purpurea extracts. J Chromatogr A 1103(2), 219-228.
Cech, N. B., Kandhi, V., Davis, J. M., Hamilton, A., Eads, D., Laster, S. M., 2010. Echinacea and its alkamides: effects on the influenza A-induced secretion of cytokines, chemokines, and PGE(2) from RAW 264.7 macrophage-like cells. Int Immunopharmacol 10(10), 1268-1278.
Dedkova, E. N., Sigova, A. A., Zinchenko, V. P., 2000. Mechanism of action of calcium ionophores on intact cells: ionophore-resistant cells. Membrane & cell biology 13(3), 357-368.
Feske, S., 2009. ORAI1 and STIM1 deficiency in human and mice: roles of store-operated Ca2+ entry in the immune system and beyond. Immunological Reviews 231(1), 189-209.
Finn, D. F., Walsh, J. J., 2013. Twenty-first century mast cell stabilizers. British Journal of Pharmacology 170(1), 23-37.
Gilfillan, A. M., Tkaczyk, C., 2006. Integrated signalling pathways for mast-cell activation. Nat Rev Immunol 6(3), 218-230.
Graham, A. C., Hilmer, K. M., Zickovich, J. M., Obar, J. J., 2013. Inflammatory response of mast cells during influenza A virus infection is mediated by active infection and RIG-I signaling. The Journal of Immunology 190(9), 4676-4684.
Greger, H., 2016. Alkamides: a critical reconsideration of a multifunctional class of unsaturated fatty acid amides. Phytochemistry Reviews 15(5), 729-770.
Gronich, J. H., Bonventre, J. V., Nemenoff, R. A., 1990. Purification of a high-molecular-mass form of phospholipase A₂ from rat kidney activated at physiological calcium concentrations. Biochemical Journal 271(1), 37-43.
Hoth, M., Penner, R., 1992. Depletion of intracellular calcium stores activates a calcium current in mast cells. Nature 355(6358), 353-356.
Hou, C. C., Chen, C. H., Yang, N. S., Chen, Y. P., Lo, C. P., Wang, S. Y., Tien, Y. J., Tsai, P. W., Shyur, L. F., 2010. Comparative metabolomics approach coupled with cell- and gene-based assays for species classification and anti-inflammatory bioactivity validation of Echinacea plants. The Journal of Nutritional Biochemistry 21(11), 1045-1059.
Hou, C. C., Huang, C. C., Shyur, L. F., 2011. Echinacea Alkamides Prevent Lipopolysaccharide/D-Galactosamine-Induced Acute Hepatic Injury through JNK Pathway-Mediated HO-1 Expression. Journal of Agricultural and Food Chemistry 59(22), 11966-11974.
Kindscher, K., 1989. Ethnobotany of purple coneflower (Echinacea angustifolia, Asteraceae) and Other Echinacea Species. Econ Bot 43(4), 498-507.
Klein, M., Klein-Hessling, S., Palmetshofer, A., Serfling, E., Tertilt, C., Bopp, T., Heib, V., Becker, M., Taube, C., Schild, H., Schmitt, E., Stassen, M., 2006. Specific and Redundant Roles for NFAT Transcription Factors in the Expression of Mast Cell-Derived Cytokines. The Journal of Immunology 177(10), 6667-6674.
Kuehn, H. S., Radinger, M., Gilfillan, A. M., 2010. Measuring mast cell mediator release. Current Protocols in Immunology, 7.38. 31-37.38. 39.
LaLone, C. A., Hammer, K. D., Wu, L., Bae, J., Leyva, N., Liu, Y., Solco, A. K., Kraus, G. A., Murphy, P. A., Wurtele, E. S., 2007. Echinacea species and alkamides inhibit prostaglandin E2 production in RAW264. 7 mouse macrophage cells. Journal of Agricultural and Food Chemistry 55(18), 7314-7322.
Ley, J. P., Krammer, G., Looft, J., Reinders, G., Bertram, H., 2006. Structure-activity relationships of trigeminal effects for artificial and naturally occurring alkamides related to spilanthol. Developments in Food Science 43, 21.
Leyte-Lugo, M., Todd, D. A., Gulledge, T. V., Juzumaite, M., Carter, F. S., Laster, S. M., Cech, N. B., 2015. Cytokine-Suppressive Activity of a Hydroxylated Alkamide from Echinacea purpurea. Planta Med Lett 2(01), e25-e27.
Liu, C., Au, J. D., Zou, H. L., Cotten, J. F., Yost, C. S., 2004. Potent Activation of the Human Tandem Pore Domain K Channel TRESK with Clinical Concentrations of Volatile Anesthetics. Anesthesia & Analgesia 99(6), 1715-1722.
Luik, R. M., Wu, M. M., Buchanan, J., Lewis, R. S., 2006. The elementary unit of store-operated Ca²⁺ entry: local activation of CRAC channels by STIM1 at ER-plasma membrane junctions. The Journal of Cell Biology 174(6), 815-825.
Moazami, Y., Gulledge, T. V., Laster, S. M., Pierce, J. G., 2015. Synthesis and biological evaluation of a series of fatty acid amides from Echinacea. Bioorganic & Medicinal Chemistry Letters 25(16), 3091-3094.
Olah, A., Szabo-Papp, J., Soeberdt, M., Knie, U., Dahnhardt-Pfeiffer, S., Abels, C., Biro, T., 2017. Echinacea purpurea-derived alkamides exhibit potent anti-inflammatory effects and alleviate clinical symptoms of atopic eczema. J Dermatol Sci.
Ottea, J. A., Payne, G. T., Soderlund, D. M., 1990. Action of insecticidal N-alkamides at site 2 of the voltage-sensitive sodium channel. Journal of Agricultural and Food Chemistry 38(8), 1724-1728.
Passante, E., Frankish, N., 2009. The RBL-2H3 cell line: its provenance and suitability as a model for the mast cell. Inflammation Research 58(11), 737-745.
Pawankar, R., Canonica, G., Holgate, S., Lockey, R., 2013. The World Allergy Organization (WAO) White Book on Allergy (Update).
Raduner, S., Majewska, A., Chen, J. Z., Xie, X. Q., Hamon, J., Faller, B., Altmann, K. H., Gertsch, J., 2006. Alkamides from Echinacea are a new class of cannabinomimetics Cannabinoid type 2 receptor-dependent and -independent immunomodulatory effects. Journal of Biological Chemistry 281(20), 14192-14206.
Rivera, J., Fierro, N. A., Olivera, A., Suzuki, R., 2008. New Insights on Mast Cell Activation via the High Affinity Receptor for IgE1, Advances in Immunology. Academic Press, pp. 85-120.
Sharma, M., Anderson, S. A., Schoop, R., Hudson, J. B., 2009. Induction of multiple pro-inflammatory cytokines by respiratory viruses and reversal by standardized Echinacea, a potent antiviral herbal extract. Antiviral Research 83(2), 165-170.
Smyth, J. T., DeHaven, W. I., Bird, G. S., Putney, J. W., 2007. Role of the microtubule cytoskeleton in the function of the store-operated Ca²⁺ channel activator STIM1. Journal of Cell Science 120(21), 3762-3771.
Spelman, K., Iiams-Hauser, K., Cech, N. B., Taylor, E. W., Smirnoff, N., Wenner, C. A., 2009. Role for PPARγ in IL-2 inhibition in T cells by Echinacea-derived undeca-2E-ene-8,10-diynoic acid isobutylamide. Int Immunopharmacol 9(11), 1260-1264.
Sugai, E., Morimitsu, Y., Iwasaki, Y., Morita, A., Watanabe, T., Kubota, K., 2005. Pungent Qualities of Sanshool-Related Compounds Evaluated by a Sensory Test and Activation of Rat TRPV1. Bioscience, Biotechnology, and Biochemistry 69(10), 1951-1957.
Sutovska, M., Capek, P., Kazimierova, I., Pappova, L., Joskova, M., Matulova, M., Franova, S., Pawlaczyk, I., Gancarz, R., 2015. Echinacea complex-chemical view and anti-asthmatic profile. J Ethnopharmacol 175, 163-171.
Talley, E. M., Bayliss, D. A., 2002. Modulation of TASK-1 (Kcnk3) and TASK-3 (Kcnk9) Potassium Channels: Volatile Anesthetics and Neurotransmitters Share a Molecular Site of Action. Journal of Biological Chemistry 277(20), 17733-17742.
Todd, D. A., Gulledge, T. V., Britton, E. R., Oberhofer, M., Leyte-Lugo, M., Moody, A. N., Shymanovich, T., Grubbs, L. F., Juzumaite, M., Graf, T. N., Oberlies, N. H., Faeth, S. H., Laster, S. M., Cech, N. B., 2015. Ethanolic Echinacea purpurea Extracts Contain a Mixture of Cytokine-Suppressive and Cytokine-Inducing Compounds, Including Some That Originate from Endophytic Bacteria. PLoS ONE 10(5), e0124276.
Tsien, R. Y., 1980. New calcium indicators and buffers with high selectivity against magnesium and protons: design, synthesis, and properties of prototype structures. Biochemistry 19(11), 2396-2404.
Tsunozaki, M., Lennertz, R. C., Vilceanu, D., Katta, S., Stucky, C. L., Bautista, D. M., 2013. A ‘toothache tree’alkamide inhibits A mechanonociceptors to alleviate mechanical pain. The Journal of physiology 591(13), 3325-3340.
Vig, M., DeHaven, W. I., Bird, G. S., Billingsley, J. M., Wang, H., Rao, P. E., Hutchings, A. B., Jouvin, M.-H., Putney, J. W., Kinet, J.-P., 2008. Defective mast cell effector functions in mice lacking the CRACM1 pore subunit of store-operated calcium release-activated calcium channels. Nat Immunol 9(1), 89-96.
Vig, M., Kinet, J.-P., 2009. Calcium signaling in immune cells. Nat Immunol 10(1), 21-27.
Wernersson, S., Pejler, G., 2014. Mast cell secretory granules: armed for battle. Nat Rev Immunol 14(7), 478-494.
Woelkart, K., Bauer, R., 2007. The Role of Alkamides as an Active Principle of Echinacea. Planta Med 73(07), 615-623.
Woelkart, K., Linde, K., Bauer, R., 2008. Echinacea for Preventing and Treating the Common Cold. Planta Med 74(06), 633-637.

### Example 2. Alkamides and their Effects on Pain and Itch

Alkamides are a class of small molecule produced by a number of different plants. Each alkamide contains a fatty acid of approximately 10-20 carbons, an amide, and a head group. FIG. 10 below shows the structure of the alkamide dodeca-2E,4E-dienoic acid isobutylamide which is produced by the plant Echinacea purpurea. Also shown in FIG. 10 is a synthetic alkamide known as YM8-85 where the fatty acid chain is four carbons shorter in length.

With mast cells, A15 prevents the rise in intracellular calcium necessary for this cell to undergo degranulation. As a result, A15 inhibited the release of allergic and inflammatory mediators. Calcium responses are also critical to activation of neurons and for the physiological responses of pain and itch. In this example, experiments were undertaken to determine whether the alkamides A15 and YM8-85 inhibit neuron activation leading to inhibition of pain and itch.

### Methods

Several different model systems were utilized for these experiments. In one model, dorsal root ganglia were obtained from mice and the neurons cultured in vitro. The neurons were then stimulated in the presence/absence of A15 and YM8-85 and the calcium responses monitored by fluorescence microscopy. In the second model, the alkamides were applied to mouse skin in mineral oil. The reactions of these mice to heat, cold, and tactile stimulation was then compared with mice that had not received treatment with A15. In a third model, mice were sensitized to the chemical compound toluene-2,4-diisocyanate (TDI). TDI is an industrial chemical which causes hypersensitive skin lesions and itching in exposed humans and animals. The physiological response to TDI is complex involving type 2 T lymphocytes, cytokines, mast cells, and neurons. Alkamides were applied to sensitized animals and then ear swelling and bouts of scratching were monitored.

### Results and Discussion

In vitro DRG neuron responses. The receptors TRPA1 and TRPV1 are expressed on neurons and are known to mediate responses to environmental stimuli. TRPA1 mediates responses to heat, cold, and itch while TRPV1 mediates responses to very high temperatures and pain. Experiments were performed to determine whether A15 and YM8-85 could inhibit responses mediated by these receptors. Neurons were stimulated with mustard (allyl isothiocyanate) (A) an agonist for the receptor TRPA-1 or capsaicin (B) an agonist for the receptor TRPV-1 and calcium responses were measured in individual neurons by fluorescence microscopy. Both A15 and YM8-85 were found to inhibit the DRG response to mustard (A) while A15 but not YM8-85 inhibited the response to capsaicin (B). Apparently, for blocking TRPA1 responsiveness, the structural differences between A15 and YM8-85 are not significant. On the other hand, the longer fatty acid chain of A15 appears to be critical to its ability to block TRPV1-mediated responses.

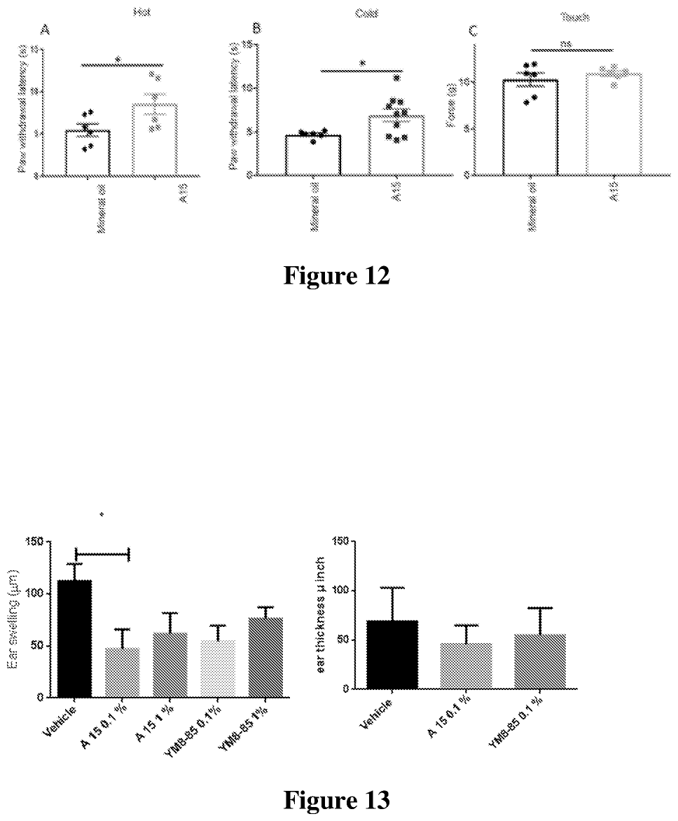

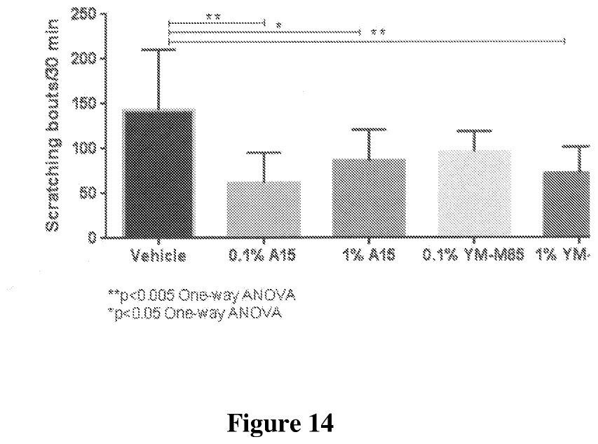

In vivo responses to painful stimuli. A15 was tested with mice to determine whether the in vitro inhibitory responses noted in FIG. 11 are also seen in vivo. A15 was mixed in mineral oil and added to mouse paws prior to stimulation with heat, cold, or touch. As shown in FIG. 12, it was found that A15 increased withdrawal latency to both heat (A) and cold (B) the effects of A15 on TRPA1 and TRPV1 are indeed seen in vivo and that A15 is indeed reducing painful signaling from these receptors. On the other hand, A15 did not affect touch latency. Sensations of touch are mediated by distinct families of mechanoreceptors which are apparently unaffected by A15.

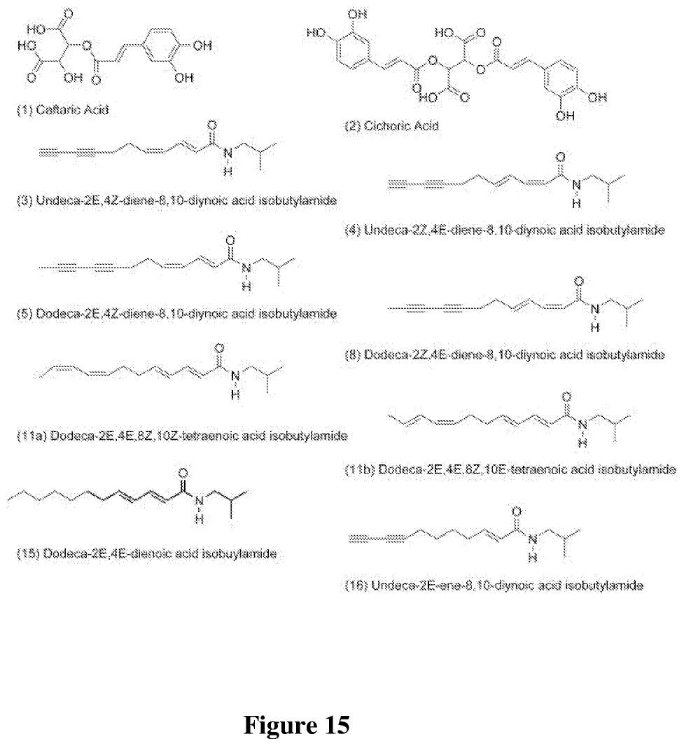

A15 and TDI induced hypersensitivity. Mice were sensitized to toluene-2,4-diisocyanate (TDI) via abdominal application. Subsequently, a hypersensitive response was induced in the ear by direct application of TDI and ear thickness measured as a sign of inflammation. As shown in FIG. 13A, a significant inhibitory effect was noted with 0.1% A15. However, this response was not very strong (W. Baeumer, personal communication) and significant responses were not seen with 1% A15 nor with YM8-85 at either dosage. The experiment was repeated and neither A15 nor YM8-85 at 0.1% produced significant inhibition of ear swelling (FIG. 13B). It was concluded that A15 and YM8-85 do not inhibit the hypersensitive inflammation induced by TDI.

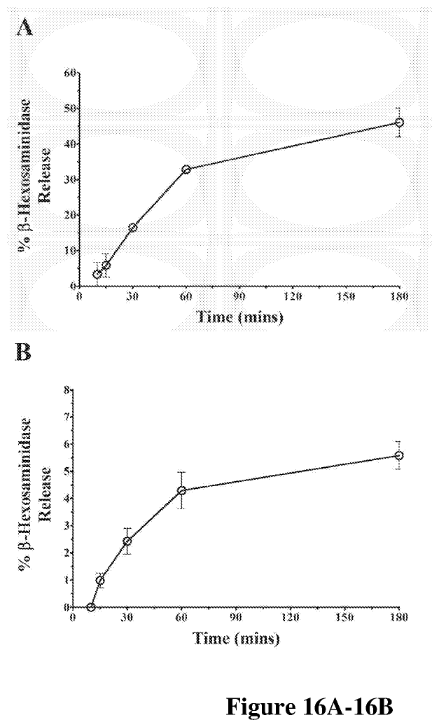

The effects of A15 on TDI-induced itch. The itch response was also monitored in mice sensitized and treated with TDI. Treated mice were video recorded and the number of scratching bouts quantified by an observer. As shown in FIG. 14, these measurements revealed strong significant inhibition of scratching by mice treated with A15 at both doses tested. Treatment with the higher dose of YM8-85 (1.0%) also revealed significant inhibition of scratching while the lower dose (0.1%) did not. Taken together these results show that alkamides such as A15 are useful as a topical agent to reduce the itch response.

### Example 3. Synthetic Alkamide 15 and the Structural Components for Mast Cell Inhibition

A number of medicinal plants produce fatty acid amides, also referred to as alkamides. These molecules have been shown to exert effects on a variety of cell types and have been linked to the health effects of medicinal plants. In this example, the effects of alkamides on the function of mast cells was investigated. Mast cells are responsible for many of the symptoms associated with allergic reactions, including asthma and atopic dermatitis. A set of alkamides were used based on the structure of dodeca-2E, 4E-dienoic acid isobutylamide, also known as A15, which is produced by the plant Echinacea purpurea. Specifically, the level of saturation and the length of the fatty acid that produced superior inhibition of mast cell degranulation was determined. The two double bonds in this alkamide were dispensable for the activity toward mast cells. Regarding the length of the fatty acid chain, with both IgE- and ionophore-induced degranulation, inhibition was best achieved with a fatty acid of 8-12 carbons. Significant inhibition of calcium signaling was also achieved by an alkamide with an 8-12 carbon fatty acid chain. Together, these data show that alkamides inhibit mast cell degranulation by suppressing calcium responses. Finally, the effects of alkamides on the secretion of the cytokine TNF-α was investigated. In this case, superior inhibition was achieved by alkamides with fatty acids of 10-12 carbons, demonstrating different mechanisms for alkamide-mediated inhibition of degranulation and de novo cytokine synthesis.

### Abbreviations

AIT: Allergen immunotherapy AG: Antigen; ITAM: Immunoreceptor tyrosine-based activation motifs LAT: Linker for activation of T cells; PIP₂: Phosphatidylinositol-4,5-bisphosphate DAG: Diacylglycerol; IP₃: Inositol 1,4,5-triphosphate ER: Endoplasmic reticulum; CRAC: Calcium release-activated calcium channel GPCR: G-protein coupled receptors; SNARE: N-ethylmaleimide-sensitive factor attachment protein receptor PI3K: Phosphatidylinositol 3-kinase; DNP: Dinitrophenyl; p-NAG: p-nitrophenyl-N-acetyl-3-D-glucosaminide STIM 1: Stromal Interaction Molecule 1; SOAR: STIM1 Orail-activating region PMA: Phorbol 12-myristate 13-Acetate PHA: Phytohemagglutinin; TMB-8: 3,4,5-trimethoxybenzoic acid 8-(diethylamino) octyl ester TNF-α: Tumor necrosis factor alpha.

### BACKGROUND

### Echinacea and its Uses

Echinacea is a perennial plant of the Asteraceae family found native to the United States and Canada. The most common species, Echinacea angustifolia, Echinacea pallida, and Echinacea purpurea are geographically distributed throughout the Great Plains region of the United States and Canada. E. angustifolia and E. pallida are native to both countries while E. purpurea, also known as purple coneflower, was originally native to the US and introduced into Canada during the mid-1900's (Desmet & Brouilet, 2013; Kindscher, 1989; Shadow, 2017; Stevens, 2017).

Knowledge about the health benefits of Echinacea was obtained primarily, from various Indian tribes and their uses of the plant. The Sioux Nation (Dakota and Lakota tribes), and the Meskwaki, Kiowa, and Choctaw tribes, used Echinacea preparations for a variety of medical conditions including, sore throats, colds and flu, and venomous bites. During the early 1900's Echinacea was adopted by the broader American and European cultures with extracts used to treat colds and flu, atopic dermatitis, ulcers, bacterial infections, and to promote wound healing (Borchers, Keen, Stern, & Gershwin, 2000; Flannery, 1999; Kindscher, 1989; Smith, Huron H (Huron Herbert), 1928).

There are two hypotheses for the medicinal effects of Echinacea. Early on, investigators postulated that Echinacea preparations provided a health benefit by promoting clearance of infections through augmentation or enhancement of the immune system (Flannery, 1999).

Support for this hypothesis has come from several clinical trials where daily usage of Echinacea reduced upper respiratory tract infections, compared to the placebo, in adults and children (Schapowal, Klein, & Johnston, 2015; Weber et al., 2005). For example, reduced levels of a panel of respiratory viruses were reported in a trial where Echinacea was used to treat patients with recurrent colds and chronic respiratory symptoms (Jawad, Schoop, Suter, Klein, & Eccles, 2012). An alternative hypothesis for the medicinal effects of Echinacea emerged when the role of cytokines in the symptoms of infectious and inflammatory disease became known. This hypothesis postulates that Echinacea suppresses release or action of inflammatory mediators thereby reducing the symptoms of disease. In support of this hypothesis, a number of the constituents of Echinacea have been shown to suppress production of pro-inflammatory mediators from immune cells in vitro (Cech et al., 2006; Jeon et al., 2015; Ramasahayam et al., 2011; Spelman, Wetschler, & Cech, 2009; Todd et al., 2015).

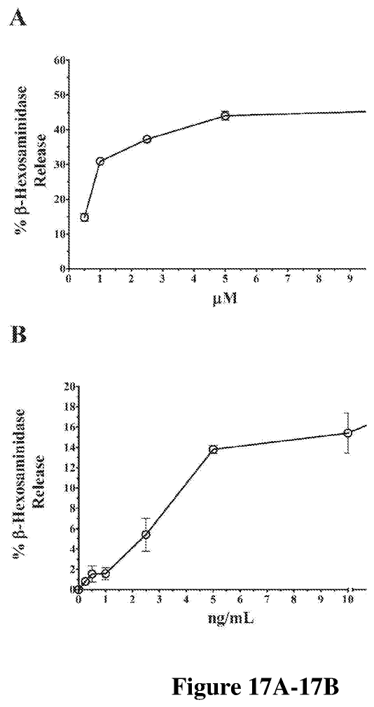

The Chemical Components of Echinacea and Their Effects Both Echinacea roots and the aerial components of the plant are harvested and used as herbal medicines. Aerial components are typically extracted with hot water (aqueous extraction) and can include a low percentage of ethanol (i.e., 20%). These extracts typically contain high concentrations of caffeic acid, glycoproteins, and polysaccharides; components that have been associated with the immune stimulating benefits of Echinacea (Benson et al., 2010; Cech et al., 2006; Sasagawa, Cech, Gray, Elmer, & Wenner, 2006; Spelman et al., 2009) (FIG. 15). Several studies have been performed with purified compounds from aerial extracts that demonstrate immune stimulating activity. For example, a study with arabinogalactan, a polysaccharide that consists of a galactan backbone with attached arabinose side chains linked with rhamnose showed increased phagocytic activity and secretion of TNF-α from macrophages (Ferreira, Passos, Madureira, Vilanova, & Coimbra, 2015; Luettig, Steinmiller, Gifford, Wagner, & Lohmann-Matthes, 1989). In addition, arabinogalactan-containing proteins isolated from Echinacea stimulated greater nitric oxide production and IL-6 secretion in murine macrophages (Classen, Thude, Blaschek, Wack, & Bodinet, 2006). Finally, arabinogalactan isolated from larch wood was shown to stimulate the immune system by increasing the numbers of NK cells and mature granulocytes isolated from the spleen of mice (Currier, Lejtenyi, & Miller, 2003).

In contrast, Echinacea roots are typically extracted with a high concentration of ethanol (75-95%). These extracts are typically high in alkamides and caffeic acids, with minor amounts of polyphenols, cichoric acids, and glycoproteins (FIG. 15) and have been associated with suppression of inflammation and cytokine mediated symptoms (Benson et al., 2010; Cech et al., 2006; Ramasahayam et al., 2011; Spelman et al., 2009). A number of laboratories have documented the ability of alkamides to suppress production of inflammatory mediators and these results are described in the section below. Caffeic acid has also been shown suppress secretion of TNF-α from HMC-1 cells (Jeon et al., 2015).

Despite several tantalizing in vitro studies, clinical trials with Echinacea extracts have been ambiguous. The variety of different plant structures and methods which are used to produce extracts from Echinacea may contribute to the inconsistent and confusing results of clinical trials. Cech et al., found that different habitats and plant age were factors that contributed to variable concentrations of alkamides, caftaric acid and cichoric acid produced by Echinacea (Cech et al., 2010). Additionally, investigators attribute successful results of clinical trials to immunologically stimulatory properties of Echinacea without properly performing and interpreting clinical measurements and assays to support these claims (B. P. Barrett, Brown, Locken, Maberry, & al, 2002; Jawad et al., 2012; Taylor et al., 2003; Weber et al., 2005). For example, Jawad et al., provided data analyzing viral load in nasal swabs of each participant. However, data corresponding to the effect of E. purpurea on the immune response, including serum antibody concentration, serum cytokine levels, and immune cell numbers were not provided. Barrett et al., suggested that results of Echinacea clinical trials were inconsistent, in part, because of a participant belief in the benefits of Echinacea and a placebo effect. These factors were suggested to skew the severity and duration of cold symptoms towards a favorable outcome for Echinacea treatments (B. Barrett et al., 2011).

The method of Echinacea extract preparation differs between clinical studies. For example, Jawad et al., used Echinacea ethanolic extracts of aerial (95%) and root (5%) structures, which contain mostly alkamides, and found that the number of days until secondary upper respiratory infections (URI) was increased in participants that used this preparation (Jawad et al., 2012). In contrast, Taylor et al., used Echinacea aqueous extracts of aerial structures, which contain mostly polysaccharides and glycoproteins, and found that participants that used this extract did not delay the time until secondary URIs (Taylor et al., 2003). The concentration of constituents contained in each E. purpurea preparation was not accounted for in these clinical trials, contributing to discrepancies in constituent dosing. Jawad et al., used doses of E. purpurea that were standardized based on the alkamide dodecatetraenoic acid isobutylamide, in which the daily doses varied from 2.7 mL to 4.5 mL (0.12 to 0.20 mg of dodecatetraenoic acid isobutylamide). Taylor et al., used the aqueous preparation and the daily dose varied from 7.5 mL to 10 mL with no constituent standardization performed and no measurement of constituent concentration per dose. The inconsistent study parameters mentioned above illustrate the need for more control in clinical trials to accurately assess the effectiveness of E. purpurea for the prevention and treatment of colds.

### The Alkamides

Alkamides consist of an amide head group connected to a fatty acid (FIG. 15). The fatty acid may vary in length and also in degree of saturation. Furthermore, alkamides may contain alkyl, thienyl, hydroxyl or piperdine groups attached to the amide head group or fatty acid chain. Several species of the Asteraceae family produce alkamides, including Echinacea angustifolia, Echinacea purpurea, Echinacea pallida, Acmella oleracea, Anacyclus pyrethrum, and Otanthus maritimus (Boonen, Sharma, Dixit, Burvenich, & Spiegeleer, 2012; Cheng et al., 2015; Cruz et al., 2016; Nomura et al., 2013; Ruiu et al., 2013). As mentioned above, high concentrations of alkamides are found in ethanolic extracts of Echinacea (Spelman et al., 2009; Cech et al., 2010).

Several studies have shown that alkamides can suppress the production of inflammatory mediators. For example, Echinacea extracts with high levels of alkamides suppressed LPS induced TNF-α secretion in human peripheral blood monocytes and macrophages (Gertsch, Schoop, Kuenzle, & Suter, 2004). Alkamides also suppressed LPS induced nitric oxide production in macrophages (Chen et al., 2005). Furthermore, PMA/PHA induced IL-2 production in Jurkat T cells was inhibited by alkamides (Sasagawa et al., 2006; Spelman et al., 2009). Alkamides also inhibited TNF-α and PGE2 secretion in influenza A stimulated macrophages (Cech et al., 2010). Finally, TNF-α secretion was inhibited in LPS stimulated RAW 264.7 cells treated with Echinacea extracts containing high levels of alkamides (Todd et al., 2015).

Recently, Moazami et al. examined the relationship between alkamide structure and activity (Moazami, Gulledge, Laster, & Pierce, 2015). To synthesize alkamides, the authors performed a series of oxidation steps to yield the carboxylic acid, (2E,4E)-dodeca-2,4-diezoic acid, from the commercial compound, (2E,4E)-dodeca-2,4-dien-1-ol. Finally, the resulting carboxylic acid was coupled with the isobutyl amine, propylphosphonic anhydride, to yield A15. Variants of A15 were produced using similar oxidation/coupling reactions, but differed in starting and coupling compounds. The resulting compounds were then tested for their effects on the production of TNF-α by RAW 264.7 macrophage-like cells. These studies showed that any change in the in the length of the fatty acid from the natural product, either shorter or longer, resulted in reduced inhibition of LPS stimulated TNF-α production from RAW 264.7 cells.

Similarly, changes in the structure of the amide head group cells also lead to reduced inhibitory activity. In contrast, altering the number double bonds did not affect activity (Moazami et al., 2015). The relatively simple ability to modify the structure of alkamides may lead to improvements in their specific activity and effectiveness to treat inflammatory conditions.

### Allergic Disease

Allergy is a world-wide problem and millions of people suffer from allergies to foods, drugs, animal dander, and plant pollens. Allergies can take a number of clinical forms including allergic rhinitis, atopic dermatitis, food allergies, and asthma to name just a few. A number of studies indicate that up to 20% of the population can suffer from allergy. Food and drug allergies can affect 7-8% of the population (Schwartz, Eberle, & Voehringer, 2016). Finally, the prevalence of allergic rhinitis is up to 40% and has remained relatively constant in the United States since 1997 (Eifan & Durham, 2016; Scadding et al., 2017).

Allergic reactions are immune reactions that develop following crosstalk between dendritic cells, T and B lymphocytes, and mast cells. Initially, an allergen is encountered by antigen presenting cells, processed, and peptides are presented to T-lymphocytes. Subsequently, the T cells produce cytokines including IL-4 which drive B lymphocyte production of Ag-specific IgE. The IgE in turn binds to the a subunit of the high affinity FcεRI receptor expressed on mast cells, sensitizing these cells. Once allergen binds to the surface bound IgE, the mast cells become activated, resulting in the exocytosis and the release of mediators into the extracellular space through a process termed degranulation. Mast cells release dozens of biologically active molecules, including histamine, serotonin, chymase, tryptase, and cytokines such as TNF-α. These molecules exert a number of effects on the local tissue environment. For example, tryptases and chymases released from granules can lead to remodeling of the surrounding tissue, while release of histamine and serotonin leads to increased vasculature permeability and extravasation of immune cells. (Saffar et al., 2007; Donnadieu, Jouvin, & Kinet, 2000; Blank et al., 2014).

In addition to the release of pre-formed mediators that are stored in mast cell granules, mast cells initiate the de novo synthesis of eicosanoids and cytokines and these molecules contribute the symptoms of allergic disease. For example, release of eicosanoids, including leukotrienes and prostaglandins, causes constriction of airway smooth muscle. Cytokine and chemokines exert many activities on the local tissues, and in addition, result in the activation and recruitment other immune cells (Blank et al., 2014; Lundequist & Pejler, 2011).

### The Mast Cell Activation Pathway

The mast cell is a highly granulated type of immune cell whose activity is critical for allergic reactions. Mast cells are found throughout the body and labelled by their anatomic location, i.e. serosal mast cells, mucosal mast cells, and connective tissue mast cells, while a circulating form of the mast cell is known as the basophil. Mast cells are activated by crosslinking IgE bound to their FcεRI receptors, resulting in the release of both preformed, and mediators synthesized through de novo pathways. Due to their clinical importance, with an eye towards development of inhibitors, the biochemical signaling pathways in mast cells have been studied extensively.

Initially, following the cross-linking of surface IgE, the Lyn kinase is activated by auto-phosphorylation and phosphorylates the ITAMs of β and γ subunits of the Fce receptor.

Subsequently, the tyrosine kinase, Syk, is recruited to the activated ITAMs of the γ subunit and phosphorylated by receptor associated Lyn. Activated Syk in turn phosphorylates the membrane scaffold protein, linker for activation of T cells (LAT), allowing PLC-γ to associate (Huber & Gibbs, 2015; Lin, Huang, Huang, Tzeng, & Lin, 2016). Subsequently, PLC-γ cleaves phosphatidylinositol 4,5-bisphosphate (PIP₂) leading to production of diacylglycerol (DAG) and inositol 1,4,5-trisphosphate (IP₃).

The next phase of mast cell activation centers on the second messenger calcium, which plays a critical role in mast cell mediator release. Calcium concentrations in mast cell cytoplasm are elevated in a two-step process. Initially, IP₃ formed from the cleavage of PIP₂ by PLC-γ, binds to the IP₃ receptor located at the endoplasmic reticulum (ER), causing release of calcium from the ER into the cytoplasm. Next, the ER sensor protein STIM 1, senses the reduction in ER calcium concentration and relocates near the plasma membrane binding ORAI 1 and causing activation of the calcium release-activated calcium (CRAC) channel. Finally, calcium enters from the extracellular space through the open CRAC channel (Huber & Gibbs, 2015; A. H. Shim, Tirado-Lee, & Prakriya, 2015).

The physical process of granule movement and fusion with the plasma membrane also relies on calcium. Calcium binds to the calcium domains of Rab effectors which facilitate the docking of Rab GTPases and the N-ethylmaleimide-sensitive factor attachment protein receptors (SNAREs) that are associated with secretory granules. Calcium drives these complexes to undergo granule-granule and granule-plasma membrane fusion, which is critical for degranulation to occur (Azouz, Matsui, Fukuda, & Sagi-Eisenberg, 2012; Blank & Rivera, 2004; Blank et al., 2014; Fukuda, 2013).

Signals for activation of the de novo cytokine synthesis pathway also emanate from the FcεRI receptor. The rise of intracellular calcium along with DAG formation trigger the activation of several protein kinase C isomers, whose activity is required for cytokine release (Nechushtan & Razin, 2001). Subsequently, the MAP kinase signaling cascade is activated, resulting in the transcription of cytokine related genes (Blank et al., 2014; Vonakis et al., 2005). Also, calcium binding to calcineurin leads to NFAT dephosphorylation and activation of this transcription factor. Another pathway activated in mast cells begins with the activation of phosphoinositide 3-kinase (PI3K) followed by activation of the AKT-mTOR pathway for cytokine production (Blank et al., 2014). As mentioned above extensive pharmaceutical research has been conducted on these pathways with the goal of developing inhibitory compounds.

Upstream portions of the degranulation pathway have been targeted, such as the Lyn kinase and calcium binding and channel proteins. The protein kinases responsible for the de novo synthesis pathway have also been targeted for inhibitor design (Cho, Woo, Yoon, & Kim, 2004; Diaz-Flores et al., 2013; Vuong et al., 2015).

### Treatments for Allergy

Therapeutic treatments for allergy generally focus on reducing the severity of the symptoms. Antihistamines, including azelastine and ketotifen, are histamine receptor antagonists that block the activity of histamine (Bielory, Buddiga, & Bigelson, 2004). Antihistamines temporarily resolve allergy related symptoms but usefulness is limited by the side effects, including drowsiness and dehydration. Antihistamines also only target one of the mediators of allergy and the activities of other mediators remains unaffected (Bielory et al., 2004; Tan, Sugita, & Akdis, 2016; Zhang, Finn, Barlow, & Walsh, 2016).

Glucocorticoid hormones are corticosteroids also used for the treatment of allergy. These molecules can diffuse across the plasma membrane and bind glucocorticoid receptors in the cytoplasm. The ligand and receptor glucocorticoid complex crosses the nuclear membrane to activate or suppress transcription factors associated with degranulation and cytokine production (Liu et al., 2013; Stahn & Buttgereit, 2008; Trevor & Deshane, 2014). Furthermore, there is evidence of glucocorticoid treatment that is independent of the impact on transcription factors and involves activation of kinases (Stahn & Buttgereit, 2008). The use of glucocorticoids is also limited by their side effects. Use of glucocorticoids at high doses for long periods causes negative steroid related effects, including osteoporosis, diabetes, and glaucoma (R. Gupta & Fonacier, 2016; Liu et al., 2013).

β-adrenergic agonists target β-2 receptors and has been shown to inhibit mast cell mediator release. These molecules may be used in combination with other treatments, such as corticosteroids, to treat asthma (Catalli et al., 2014). The usage of long acting μ-2 receptor agonists has been shown to be beneficial to treat severe asthma outbreaks, though long term use is considered hazardous by the FDA (Butler et al., 2016).

Monoclonal antibodies are also in use for the treatment of allergy. Monoclonal antibodies have been developed to treat allergic reactions by targeting specific molecules such as cytokines and IgE. For example, Omalizumab is a humanized monoclonal antibody that binds the Fc region of IgE, lowers available serum IgE levels, and reduces chances of mast cell mediate allergic responses. Though omalizumab has been effective, frequent costly doses are required to maintain efficacy and anaphylaxis has been reported (Stokes & Casale, 2015; Tan et al., 2016).

Allergen immunotherapy (AIT) is used to stimulate the immune system to produce IL-10 and TGF-β and induce tolerance to specific allergens. IgG antibodies are also produced and bind to FCγR receptors on mast cells leading to activation of inhibitory signaling involving phosphatase activity (da Silva, Elaine Zayas Marcelino, Jamur, & Oliver, 2014). Results of AIT differ based on route of administration, type of peptide/epitope used, or a combination of both (Cuppari et al., 2014; Scadding et al., 2017). Two recent clinical trials evaluated if tolerance could be induced to peanut and grass pollen allergens after repeated challenge (Du Toit et al., 2015; Scadding et al., 2017). Peanut tolerance was demonstrated after weekly oral consumption of peanuts resulted in lower serum IgE levels, higher serum IgG4 level, and reduced wheal from peanut skin prick tests (Du Toit et al., 2015). Tolerance to grass pollen was not established after sublingual AIT resulted in no difference in serum IgE levels and nasal challenge scores (Scadding et al., 2017). These studies also revealed several problems with AIT. Despite low doses of allergen, AIT resulted in systemic adverse reactions related to hypersensitivity leading to throat closure, urticaria, and diarrhea. Also, for some individuals it took up to 45 months for tolerance to develop.

Unfortunately, current treatments for allergy are either ineffective, targeting only one of the many mediators released by mast cells, or display dangerous side effects. Allergic reactions are highly dependent on the activity of mast cells and therefore inhibitors of mast cell function have been highly sought after by the pharmaceutical industry. The alkamides are known to inhibit the activity of a number of immune cells and therefore in this report alkamides were tested for their ability to inhibit mast cell degranulation using the model cell line RBL-2H3. The length of the fatty acid chain necessary for optimum inhibition of mast cell degranulation was also examined. The results show that the alkamide A15 was an effective inhibitor of degranulation, calcium influx, and TNF-α secretion. The length of the fatty acid chain, but not the degree of unsaturation, was a crucial component in alkamide mediated inhibition of mast cell function.

### Materials and Methods

### Chemicals and Reagents

Alkamide 15 (A15) was chemically synthesized at North Carolina State University (Raleigh, N.C.) by the J. Pierce laboratory, as previously described (Moazami et al., 2015). 1 μg/mL of mouse anti-dinitrophenyl IgE (IgE-DNP), 50 ng/mL of dinitrophenyl bovine serum albumin (DNP-BSA), and p-nitrophenyl N-acetyl-β-D-glucosamide (p-NAG), 3,4,5-trimethoxybenzoic acid 8-(diethylamino)octyl ester (TMB-8) were purchased from Sigma-Aldrich (St. Louis, Mo.). Fluo-4 Direct™ Calcium Assay Kit was purchased from ThermoFisher Scientific. The mouse TNF-α ELISA kit was from eBioscience (San Diego, Calif.).

### Cell Culture

RBL-2H3 cells were obtained from Dr. Moeser of Michigan State University and ATCC. Cells were maintained in MEM supplemented with 1% non-essential amino acids, 1% sodium pyruvate, 1% antibiotics, and 15% heat inactivated fetal bovine serum at 37° C. with 5% CO₂.

The Molecular Probes Fluo-4 Direct Calcium Assay Kit was purchased from Life Technologies.

### β-Hexosaminidase Release Assay

RBL-2H3 cells (1.0×10⁵ cells) were incubated with or without 0.1 μg/mL of IgE-DNP overnight in a 96 well plate at 37° C. with 5% CO₂. The cells were washed with Tyrode's buffer and assayed with few modifications (Kuehn, Radinger, & Gilfillan, 2010). Briefly, IgE-DNP sensitized cells were stimulated 1 hour with 5 ng/mL of DNP-BSA in the absence or presence of each compound and incubated at 37° C. with 5% CO₂. For experiments using only A23187 as the stimulant, cells were washed with Tyrode's buffer and stimulated 1 hour with 1 μM A23187 in the absence or presence of each compound and incubated at 37° C. with 5% CO₂. The stimulated cells were centrifuged at 1200×g for 5 minutes. The supernatant or cell lysate (30 μL) was incubated with 3.4 mg/mL of p-NAG (10 μL) for 1 hour at 37° C. The reaction was terminated by addition of 0.2 M sodium carbonate (pH 10) and the absorbance was read at OD405 nm on a BioTek Synergy HT microplate reader. The total 3-hexosaminidase released from the cell was calculated as the difference between the OD of the supernatant and blank plus the difference between the OD of the lysate and blank. The percent β-hexosaminidase released from the cell was calculated as the difference between the OD of the supernatant and blank divided by the total β-hexosaminidase released from the cell multiplied by 100: ODsupernatant−ODblank/[(ODlysate−ODblank)+(ODsupernatant−ODblank)]*100.

### Calcium Assay

RBL-2H3 cells (1.0×10⁵ cells) were incubated overnight in a 96 well black-wall clear bottom plate with or without IgE-DNP (0.1 μg/mL) at 37° C. with 5% CO₂. The assay was performed using the Fluo-4 Direct™ Calcium Assay Kit according to the manufacturer's instruction. Briefly, cells were incubated with the Fluo-4 Direct calcium reagent loading solution for 30 minutes at 37° C. with 5% CO₂ then 30 mins at room temperature. Prior to stimulation, the well baseline fluorescence was measured every 5 seconds over 1 minute using a BioTek Synergy HT microplate reader with 485/20 nm excitation and 528/20 nm emission filters. Cells were stimulated with 1 μM A23187 or 5 ng/mL DNP (IgE-DNP) in the absence or presence of each compound, and the fluorescence was measured every 5 seconds over 2 minutes. The change in fluorescence (ΔRT) was calculated as the sample fluorescence minus the baseline fluorescence for each well.

### ELISA

RBL-2H3 cells (1.0×10⁵ cells) were incubated overnight in a 24 well plate at 37° C. with 5% CO₂. Cells were washed with 1×PBS and stimulated with 1 μM A23187 for 18 hrs in the absence or presence of each compound. The cells were centrifuged (1200×g, 5 min) and the supernatants were collected. The concentration of TNF-α in the supernatants were determined using the TNF-α ELISA kit.

### Statistical Analysis

Graphpad Prism software (Graphpad Software, La Jolla, Calif.) was used to perform all statistical analysis. Comparison of the means of TMB-8 with A23187 or DNP-BSA stimulated cells were performed with the student's unpaired t test. Comparison of the means of A15 or variants with A23187 or DNP-BSA stimulated cells, or comparison of the means of A15 with variants were performed using One-way ANOVA with Dunnett's post hoc test. Comparison of the relationship between the number of carbons in the fatty acid portion of the alkamide with the percent inhibition of degranulation or calcium was fit using the second order polynomial fit test.

### Results

### Induction of Mast Cell Degranulation

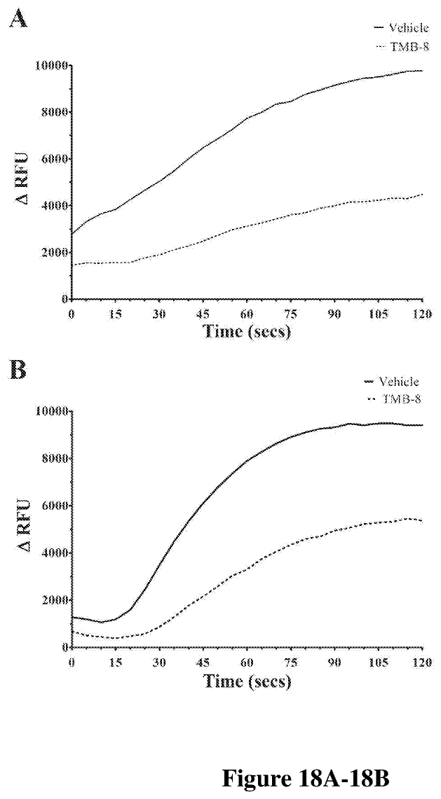

The RBL-2H3 cell line is used often to investigate the FcεRI receptor signaling events in mast cells that lead to increased intracellular calcium levels, degranulation, and cytokine production. Because RBL-2H3 cells are basophilic in origin their biochemical pathways are very similar to normal mast cells. As a marker for RBL-2H3 cell degranulation, the amount of β-hexosaminidase (β-hex) that is released into supernatant was quantified. (Kuehn et al., 2010; Passante & Frankish, 2009; Wendeler & Sandhoff, 2009). β-hex is an enzyme that cleaves carbohydrates, such as N-acetyl glucosamine (NAG). In these experiments, p-NAG was used as the substrate and measured the production of p-nitrophenol, a yellow colored product. Initially, a time course and dose response study was performed to optimize degranulation in the presence of the ionophore. Cells were plated overnight, washed, and then stimulated with 1 μM A23187 for a 3 hour period. As shown in FIG. 16A, stimulation with 1 μM A23187 resulted in the rapid release of β-hex from the cell within 1 hour of stimulation. After 1 hour, only a small increase was noted in the release of β-hex. Cells sensitized overnight with IgE anti-DNP-BSA and stimulated with 5 ng/mL DNP-BSA resulted in a similar pattern of β-hex release from the cell within 1 hour of stimulation (FIG. 16B). Overall, a lower amount of β-hex was released following stimulation with IgE-anti-BSA, compared to the amount of stimulation with 1 μM A23187.

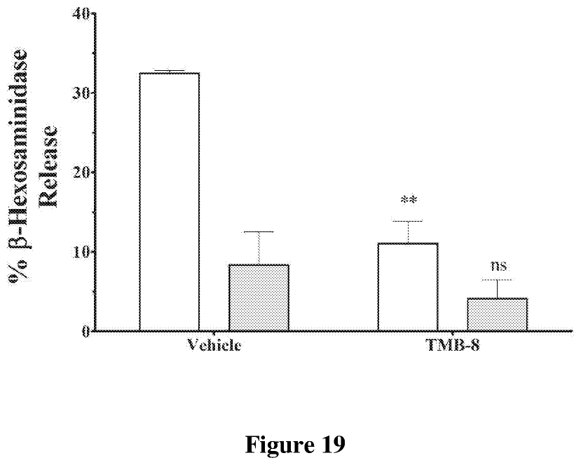

FIGS. 17A and B, respectively, display the amount of β-hex released from RBL-2H3 cells after 1 hour of stimulation with different concentrations of A23187 or DNP-BSA. Concentrations of A232187 as low as 0.5 μM induced β-hex release, while concentrations of A23187 above 2.5 μM did not cause any additional β-hex release. With DNP-BSA, concentrations as low as 0.25 ng/ml induced β-hex release, while maximum stimulation was generally observed at 5 ng/ml. Based on these results, the effects of alkamides on mast cell degranulation were evaluated using 1 μM A23187 or 5 ng/mL DNP-BSA for 1 hour.

### Calcium Dependent Mast Cell Degranulation

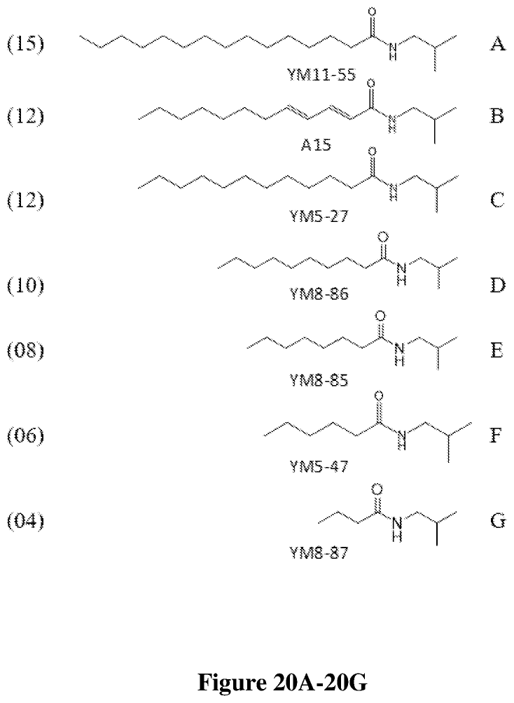

Calcium is an important component of exocytosis and degranulation in mast cells. Previous studies demonstrated that TMB-8, a calcium inhibitor, reduces the rate that extracellular calcium is imported into the cytosol (Bencherif, Eisenhour, Prince, Lippiello, & Lukas, 1995; Hu et al., 2014; Ruiz, Matute, & Alberdi, 2010). To ensure that calcium was critical in the assays used herein, the calcium antagonist TMB-8 was used. First, it was confirmed that TMB-8 did indeed inhibit the calcium response in these cells. RBL-2H3 cells were plated overnight, loaded with the calcium sensitive fluorescent dye Fluo-4 AM, and fluorescence monitored over a 2 min period. Cells stimulated with 1 μM A23187 (FIG. 18A) or 5 ng/mL DNP-BSA (FIG. 18B) displayed a rapid rise in cytosolic calcium levels. In contrast, when cells were stimulated in the presence of TMB-8, there was a decreased rate of calcium entering the cytosol and a lower fluorescence reading at the 2-minute time point, confirming that TMB-8 worked as predicted.

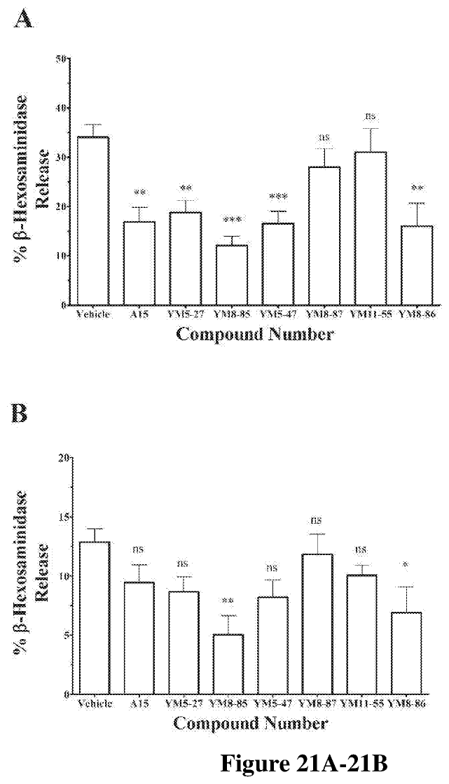

After this, degranulation was monitored in the presence of TMB-8. RBL-2H3 cells were stimulated with 1 μM A23187 or 5 ng/mL DNP-BSA in the absence or presence of TMB-8 for 1 h and the β-hex release was measured. As shown in FIG. 19, TMB-8 significantly inhibited β-hex release from A23187 stimulated cells. Release of β-hex was also inhibited by TMB-8 in DNP-BSA stimulated cells. Collectively, these findings shows that calcium is indeed a key second messenger for degranulation in RBL-2H3 cells.

### A15 and Variants Inhibit Mast Cell Degranulation and Calcium Increases

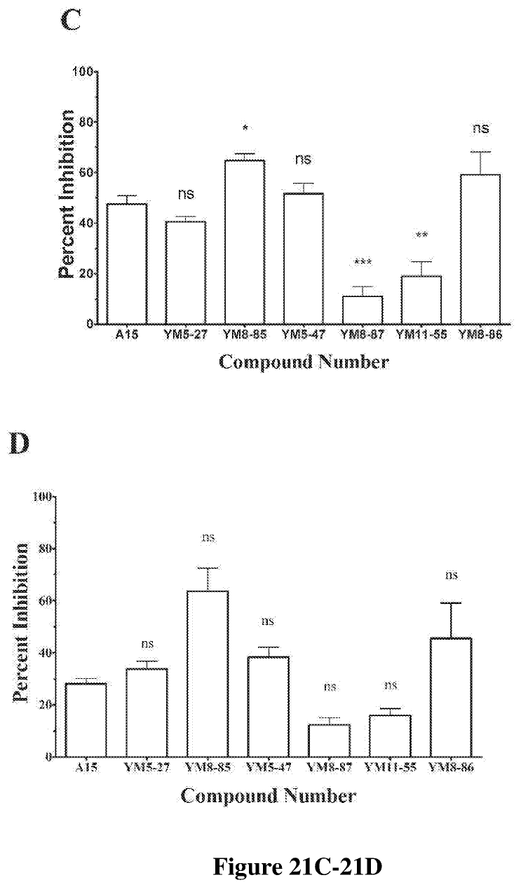

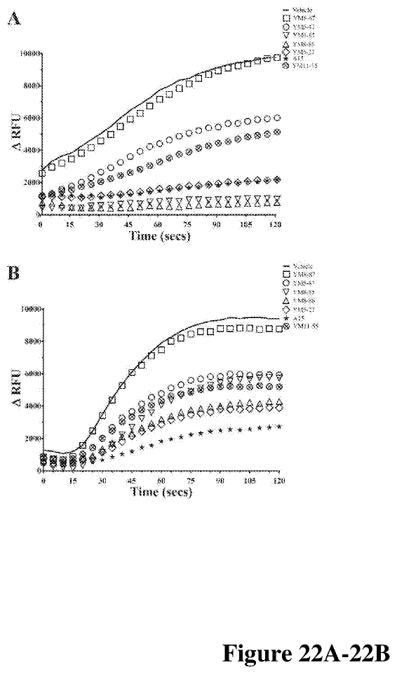

Previously, in an attempt to define the length of fatty acid chain on A15 for optimum cytokine inhibition, a series of chain length variants were synthesized and tested for effects with RAW 264.7 macrophages (Moazami et al., 2015). In this report these variants were examined for their effects on degranulation with RBL-2H3 cells. A total of 7 compounds were tested with fatty acid lengths varying from 4-15 carbons (FIG. 20). β-hex release was measured after 1 hour of stimulation with 1 μM A23187 (FIG. 21A) or 5 ng/mL DNP-BSA (FIG. 21B). The percent inhibition for each compound is shown in FIGS. 21C and D. With A23187 as the triggering agent, YM8-87 and YM11-55 were the least effective inhibitors of degranulation resulting in 11% and 18% inhibition, respectively (FIG. 21A, 21C). YM5-47, YM8-85, YM8-86, YM5-27, and A15 significantly inhibited A23187 induced degranulation resulting in 51%, 64%, 59%, 40%, and 47% inhibition, respectively. Moreover, YM8-85, which contains 8 carbons in the fatty acid, was the most effective inhibitor of degranulation.

The pattern of inhibition was very similar when DNP-BSA was used as the triggering agent (FIG. 21B, 21D). YM8-87 and YM11-55 were the least effective inhibitors of DNP-BSA induced degranulation resulting in 12% and 16% inhibition, respectively (FIG. 21D). YM5-47, YM8-85, YM8-86, YM5-27, and A15 were effective inhibitors of DNP-BSA induced degranulation resulting in 36%, 60%, 46%, 33%, and 27% inhibition, respectively. Particularly, YM8-85 and YM8-86 significantly inhibited degranulation whereas YM8-85 was the most effective inhibitor of degranulation, a finding similar to when A23187 stimulated cells were treated with each compound. When cells were treated with alkamide alone, degranulation was not induced. These data show that the fatty acid structure of the alkamide requires between 6 to 12 carbons for inhibition of mast cell degranulation. Thus, alkamides interact with a molecule that requires a particular fatty acid length.

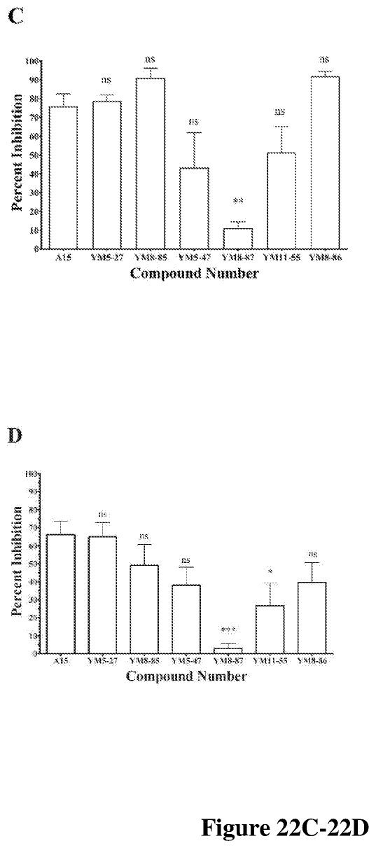

Next, it was determined if the varying ability of these compounds to inhibit degranulation stemmed from differences in their ability to inhibit the calcium response. RBL-2H3 cells were plated overnight and intracellular calcium was measured over 2 minutes by fluorescence after stimulation with 1 μM A23187 or 5 ng/mL DNP-BSA in the absence or presence of each compound. After 2 minutes of stimulation with A23187, the rapid increase of intracellular calcium levels was significantly suppressed by YM8-85, YM8-86, YM5-27, and A15 resulting in 91%, 92%, 79%, and 76% inhibition, respectively (FIG. 22A, 22C). After 2 minutes of stimulation with DNP-BSA, YM5-47, YM8-85, YM8-86, YM5-27, A15, and YM11-55 significantly suppressed the rapid increase of intracellular calcium resulting in 38%, 49%, 40%, 65%, 66%, and 27% inhibition, respectively (FIG. 22B, 22D). Consistent with degranulation, YM8-87 was the least effective inhibitor of calcium influx during A23187 and DNP-BSA stimulation. YM8-85 was the most effective inhibitor of calcium influx after stimulation with A23187, while A15 was the greatest inhibitor after DNP-BSA stimulation. When cells were treated with alkamide alone, calcium influx was not induced. These data show that the fatty acid structure of the alkamide requires between 8 to 12 carbons for optimum inhibition of intracellular calcium increases in mast cells.

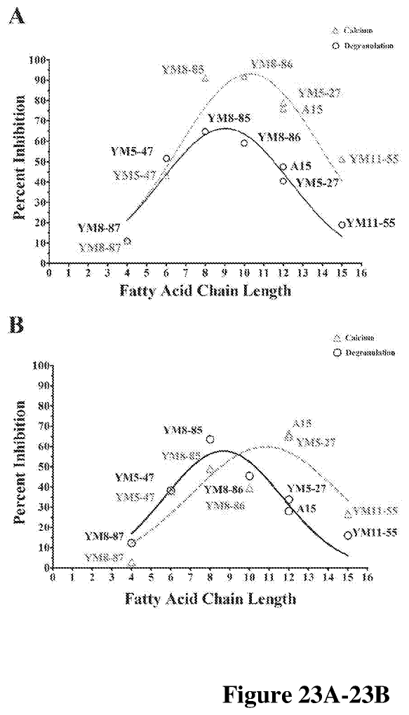

To evaluate the relationship between degranulation, calcium, and the length of alkamide fatty acid, percent inhibition data from the degranulation and calcium assays were plotted versus the number of carbons in the fatty acid for each compound. YM8-87, which has the least carbons in the fatty acid, and YM11-55, which has the most carbons in the fatty acid, were weak inhibitors of degranulation and calcium in A23187 and DNP-BSA stimulated cells (FIG. 23A, 23B). YM5-47, YM8-85, YM8-86, YM5-27, and A15, which contain fatty acids ranging from 6 to 12 carbons, were strong inhibitors of calcium and degranulation in A23187 and DNP-BSA stimulated cells. Taken together, these data show the relationship between the inhibition of degranulation and calcium during mast cell activation is dependent on an optimum length of 8 to 12 carbons in the fatty acid.

### The Alkamide Fatty Acid is Critical for Inhibition of Mast Cell TNF-α Secretion.

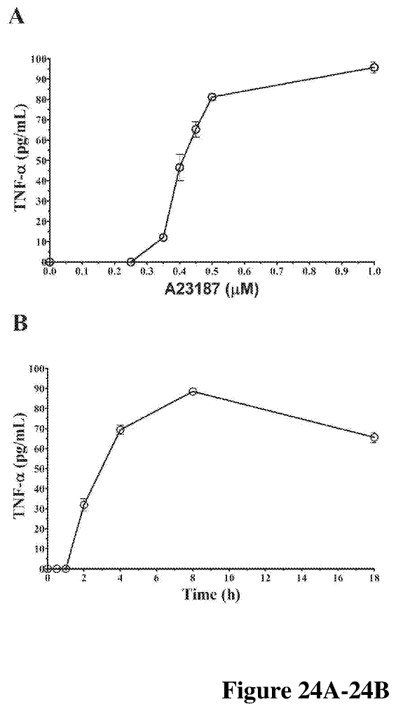

Mast cells also contribute to the symptoms of allergic disease through de novo synthesis of cytokines and alkamides have been shown to inhibit cytokine production from macrophage- and T cell-derived cell lines (Moazami et al., 2015; Spelman et al., 2009). Therefore, in these experiments, it was determined if A15 could inhibit TNF-α production from RBL-2H3 cells and, if so, whether the optimal chain length for inhibiting degranulation is also optimal for inhibiting production of TNF-α. As a first step, RBL-2H3 cells were plated overnight, stimulated with A23187, and the levels of TNF-α in the supernatants were measured by ELISA (FIG. 24). Levels of TNF-α were detected in the supernatants within 2 hours and increased up to 18 hours after stimulation (FIG. 24A). This effect was dose dependent, with increased concentrations of A23187 leading to increased levels of TNF-α production (FIG. 24B). Subsequent experiments were performed by stimulating RBL-2H3 cells with 1 μM A23187 for 18 hours.

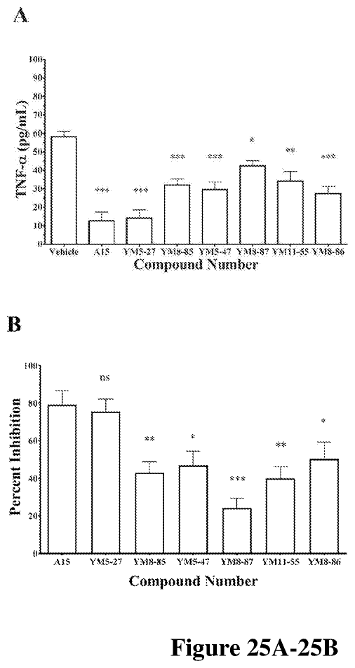

Finally, to determine whether A15 and chain length variants are inhibitors of mast cell TNF-α secretion, RBL-2H3 cells were plated overnight and stimulated with 1 μM A23187 for 18 hours in the absence or presence of each compound. FIG. 25 shows that the secretion of TNF-α in A23187 stimulated cells was significantly inhibited by YM5-47, YM8-85, YM8-86, YM5-27, A15, and YM11-55 resulting in 47%, 43%, 50%, 76%, 79%, and 40% inhibition, respectively. Alkamide treatment by itself did not induce production of TNF-α. In summary, A15 is an inhibitor of mast cell TNF-α secretion and optimum inhibition requires 10 to 12 carbons in the fatty acid. These results differ from degranulation where 8 carbons in the fatty acid were required for optimal inhibition, indicating that the alkamides affect different pathways that lead to TNF-α production and degranulation.

### DISCUSSION

Extracts from the medicinal plant Echinacea contain a class of compounds known as alkamides which several studies have shown can inhibit cytokine and eicosanoid production from monocytes, macrophages, and T lymphocytes (Gertsch et al., 2004; Chen et al., 2005; Sasagawa et al., 2006; Spelman et al., 2009; Cech et al., 2010; Todd et al., 2015). In this study, it was show that alkamides can inhibit degranulation and production of TNF-α from the RBL-2H3 basophils and the structure of the fatty acid chain of alkamide A15 that is necessary for these effects was investigated. In a previous investigation with RAW 264.7 macrophage-like cells, it was found that a minimum of 12 carbons in the fatty acid was necessary for optimum inhibition of LPS-stimulated production of TNF-α (Moazami et al., 2015). Similarly, with RBL-2H3 cells, a fatty acid chain with 12 carbons was found to be optimum. In contrast, it was found here that a fatty acid chain of 8-10 carbons was preferred for inhibition of degranulation. Together these results suggest that A15 can target distinct pathways to mediate these inhibitory effects.

During the early phase allergic response, mast cells rapidly degranulate and release mediators that contribute to the symptoms associated with asthma, allergic rhinitis, and atopic dermatitis. Inhibiting the release of these granules that contain pre-formed mediators can inhibit smooth muscle contraction, tissue remodeling, increased mucus secretion, and vasodilation (Cruse & Bradding, 2016; Galli & Tsai, 2012; Lundequist & Pejler, 2011). Mast cell degranulation is typically dependent on FcεRI crosslinking, which in our studies was modeled using anti-DNP IgE antibody and DNP-BSA. This signal leads to IP₃-dependent depletion of ER calcium stores followed by the influx of extracellular calcium. Mast cell activation by the calcium ionophore A23187 circumvents the FcεRI receptor signaling events and depletes calcium from the ER to increase the cytosolic calcium levels through influx of extracellular calcium (Han, Moon, Jeong, & Kim, 2016; Müller, Obel, Waagepetersen, Schousboe, & Bak, 2013; Na-Ra-Han, Phil-Dong-Moon, & Ka-Jung-Ryu Jae-Bum-Jang Hyung-Min-Kim Hyun-Ja-Jeong, 2016; J. Shim et al., 2016; Verma et al., 2011). Using an assay for the granule protein j-hexosaminidase, it was found that A15 inhibited mast cell degranulation in a dose dependent manner following treatment with A23187 or DNP-BSA. This finding indicates that A15 inhibits mast cell degranulation by targeting a cellular component downstream of FcεRI receptor activation but prior to the entry of extracellular calcium into the cytoplasm.

Fatty acids are important for metabolism and membrane structure (Kamp & Hamilton, 2006; Yazdi, Stein, Elinder, Andersson, & Lindahl, 2016) Fatty acids can themselves be signaling molecules (Furuhashi & Hotamisligil, 2008), or important components of more complex signaling molecules such as N-acyl ethanolamines (Divito & Cascio, 2013). N-acyl ethanolamines are typically produced in cells through the action of phospholipase D (Ezzili, Otrubova, & Boger, 2010; Leishman, Mackie, Luquet, & Bradshaw, 2016; Ogura, Parsons, Kamat, & Cravatt, 2016) and has been shown previously to inhibit receptor signaling and calcium channel activation (Divito & Cascio, 2013). Previous functional studies evaluated the impact of the double bonds and length of the fatty acid in N-acyl ethanolamines and found that increasing the number of double bonds and the length of the fatty acid resulted in greater inhibition of the calcium response (Chemin, Nargeot, & Lory, 2007). N-acyl ethanolamines with fatty acid lengths between 18-22 carbons and 2-6 double bonds inhibited calcium channels and these results were similar to the inhibitory activity of polyunsaturated fatty acids on these calcium channels (Chemin et al., 2007; Chemin, Cazade, & Lory, 2014). Mast cell calcium levels were inhibited by A15 and variants with a fatty acid that contained 6-15 carbons after stimulation with A23187 or DNP-BSA, while YM8-87 displayed the least inhibitory activity.

It was found that mast cell degranulation was inhibited by A15 and variants with a fatty acid that contained 6-12 carbons after stimulation with A23187 or DNP-BSA, while YM8-85 was the best inhibitor of both stimulants. The alkamides with the shortest (YM8-87) and longest (YM11-55) fatty acid were the least effective inhibitors of mast cell degranulation, which suggests a target site that allows medium length fatty acids to interact most effectively. Collectively, the inhibitory activity of alkamides were similar to that of N-acyl ethanolamines on the calcium channel with slight differences. In both cases, shortening the fatty acid to 2-4 carbons or lengthening it to 15-16 carbons minimized their inhibitory activity on the calcium channel. However, adding 2 double bonds in the fatty acid did not improve the activity of the alkamide on the calcium channel, whereas increasing the number of double bonds in the N-acyl ethanolamine improved calcium channel inhibition. Further modification by changing the magnitude of unsaturation to greater than 2 double bonds and increasing the length of the fatty acid to 18-22 carbons could improve the inhibitory activity on calcium channels similar to that reported for N-acyl ethanolamines.

The late phase allergic response centers on de novo synthesis and secretion of eicosanoids, chemokines, and cytokines by mast cells. These mediators have a number of effects including the recruitment of neutrophils, further exacerbating the symptoms of allergic disease (Abraham & St John, 2010; Biedermann et al., 2000). The best inhibitors of TNF-α production in RAW 264.7 cells after stimulation with LPS were A15 and YM5-27, indicating that 12 carbons in the fatty acid was optimal for inhibition (Moazami et al., 2015). In the present study, the best inhibitors of TNF-α secretion by mast cells after stimulation with A23187 were A15 and YM5-27. The similar findings, between RAW 264.7 cells stimulated by LPS and RBL-2H3 cells stimulated by A23187 or DNP-BSA, suggest that a shared molecule, involved in the transcription, translation, or secretion of TNF-α, is targeted in both cell types. Interestingly, this finding differs from degranulation where the shorter fatty acid, YM8-85, was the best inhibitor rather than A15 and YM5-27. Perhaps differences between the inhibition of degranulation and TNF-α secretion were because of the activity of calcium. It is possible that both activities result from inhibition of calcium influx, since calcium can differentially control different pathways.

For example, in signaling pathways, calcium can regulate several isoforms of PKC (Cho et al., 2004; Hosokawa et al., 2013; J. Shim et al., 2016; Swanson et al., 2016), calcineurin (Dutta et al., 2017) and PI3K/AKT (Blank et al., 2014; Divolis, Mavroeidi, Mavrofrydi, & Papazafiri, 2016), which can regulate transcription factors during cytokine production and release. Whereas degranulation requires calcium for GTPase activity and SNARE complex formation to facilitate the granule-membrane fusion process (Blank & Rivera, 2004; Blank et al., 2014). Further experiments will be necessary to discriminate between these possibilities. The examination of the TNF-α mRNA to determine whether treatment with alkamides leads to changes in its expression levels. Additionally, upstream kinase phosphorylation events or transcription factor expression levels need to be examined for changes by alkamides. To analyze the impact of alkamides on degranulation pathways, monitoring of granule trafficking and fusion events with the plasma membrane, including examination of GTPase activity and SNARE complex formation (Azouz et al., 2012; Parkinson et al., 2014).

The CRAC channel could be the specific structure that the fatty acid of the alkamide targets to prevent mast cell degranulation and suppress the calcium response. For CRAC channel activation, STIM 1 binds to the N and C terminal regions of the ORAI 1 protein, which in turn allows the formation and activation of the CRAC channel (Lewis, 2007; A. H. Shim et al., 2015; A. J. Smith et al., 2003). Investigators discovered during CRAC channel activation that STIM 1 and ORAI 1 bind via amino acid sequences located within the cytoplasmic region of each protein. The cytoplasmic region of STIM-1 contains 3 coiled-coil regions, CC1, CC2, and CC3. The STIM 1 ORAI 1-activating region (SOAR), consisting of CC2 and CC3, contains the basic amino acid sequence located within CC2 that binds to the acidic amino acid sequence of the cytoplasmic N and C terminal regions of ORAI 1. Subsequently, STIM 1 interaction with ORAI 1 causes a conformational change within the hydrophobic amino acid interior of the CRAC channel (ORAI 1 transmembrane 1) that allows the passage of calcium through the channel (Gudlur et al., 2014; Korzeniowski, Manjarrés, Varnai, & Balla, 2010; Stathopulos et al., 2013; Yamashita et al., 2017; Zhou et al., 2013). The fatty acid of the alkamide may disrupt the interaction between the cytoplasmic regions of the STIM 1 and ORAI 1 protein or disrupt CRAC channel structure to inhibit the calcium response (FIG. 16). This was previously reported when linoleic acid and steric acid disrupted ORAI 1 and STIM 1 binding by inhibiting STIM-1 clustering near the ER (Holowka, Korzeniowski, Bryant, & Baird, 2014) and when ORAI 1 c-terminal amino acid mutations disrupted the transmembrane structures of the ORAI 1 protein, affecting CRAC channel gating (Palty, Stanley, & Isacoff, 2015). It was shown here that calcium is important for mast cell degranulation, but the effect of alkamides on the calcium response should not be limited to mast cells. Neutrophils and eosinophils, two immune cells implicated in allergic disease, also undergo degranulation and depend on calcium for this process. Studies show that neutrophils and eosinophils also express CRAC channels which cause increased levels of cytosolic calcium (Clemens & Lowell, 2015; A. K. Gupta, Giaglis, Hasler, & Hahn, 2014; Nathan, 2006). In addition to mast cells, macrophages and T cells, alkamides can be useful for inhibiting the activity of these cells in pathological situations.

The activation of the immune system releases mediators that cause tissue damage and sensory pain. In addition to immune cell damage, temperature, mechanical, or chemical stimuli causes tissue damage and sensory pain, which activates additional ion channels that increase the susceptibility to hyperalgesia. The ion channels that are involved in sensory pain and function as nociceptors are the transient receptor potential (TRP) channel family. There are 6 TRP family subtypes: TRP canonical (TRPC), TRP polycystin (TRPP), TRP mucolipin (TRPML), TRP melastatin (TRPM), TRP vanilloid (TRPV) and TRP ankyrin (TRPA) (Ciardo & Ferrer-Montiel, 2017; Patapoutian, Tate, & Woolf, 2009). Activation of TRP channels in response to stimuli results in the release of calcium, but has also been shown to cause the release of potassium and sodium. The structure and function of TRP channels share some resemblance to CRAC channels. TRP channels contain six transmembrane regions, consisting of an interior pore, that are flanked by cytoplasmic N and C terminal regions with coiled-coiled domains of amino acid sequences (Ciardo & Ferrer-Montiel, 2017). Several studies report the interaction of the cytosolic terminal regions with STIM 1 (Lee et al., 2014) or calmodulin (Lau, Procko, & Gaudet, 2012), and others report that amino acid sequences in this region are necessary for channel function (Pertusa, Gonzalez, Hardy, Madrid, & Viana, 2014). The TRP channels have been implicated as therapeutic targets for the treatment of hyperalgesia and inflammation (Patapoutian et al., 2009). The mechanism of immune cell inactivation by alkamides may involve the disruption of CRAC channel activation by the fatty acid, as reported by Holowka et al., 2014 (Holowka et al., 2014). In a similar manner, alkamides may disrupt the opening of the TRP channels or, in the case of other signaling lipids and capsaicin, cause their desensitization (Ciardo & Ferrer-Montiel, 2017). This exposes a potential role for alkamides in the relief of pain caused by the response to TRP channel activation associated with osteoarthritis, rheumatoid arthritis, inflammatory bowel disease, and asthma (Ciardo & Ferrer-Montiel, 2017).

Development of therapeutics for the treatment of allergic and autoimmune diseases, such as asthma, atopic dermatitis, and rheumatoid arthritis, is ongoing. Direct targeting of the molecules responsible for the symptoms of disease is an area of recent drug discovery efforts, but a number of adverse drug events have been reported. For example, antibody therapy against IgE and TNF-α was predicted to have beneficial effects in allergy by lowering the levels of each molecule thereby reducing the associated symptoms. Specifically, a number of studies have established a relationship between the severity of asthma and increased levels of TNF-α in humans (Brown et al., 2015; Holgate et al., 2011). Consequently, studies have evaluated the efficacy of targeted treatment using anti-TNF-α antibodies to resolve symptoms of asthma (Holgate et al., 2011; Kankaanranta et al., 2014). However, treatment with etanercept did not resolve the symptoms of patients with moderate/severe asthma, illustrating the need for additional development of TNF-α inhibitors and asthma therapeutics (Holgate et al., 2011).

Additionally, montelukast, a leukotriene receptor antagonist, is used as a prophylaxis for chronic asthma and allergic rhinitis, but is ineffective during acute asthmatic attacks. Several adverse reactions including diarrhea, dyspepsia, neuropsychiatric events, and systemic eosinophilia, have also been reported after administration of montelukast and illustrate the need for safer treatment of allergic disease (Calapai et al., 2014).

Alkamides are useful as a therapeutic agent for the treatment of allergic disease. For instance, asthmatic patients inhaling aerosolized droplets of alkamides could deliver the alkamide directly to the lungs thereby reducing asthma related symptoms. An ophthalmic solution of alkamides delivered to eyes could reduce pain, itch, watering, and redness associated with pollen, ragweed, and pet allergy. Finally, topical administration of alkamides can reduce the pain and itch associated with atopic dermatitis or allergen induced urticaria. Clinical application of alkamides offers a therapeutic strategy to provide rapid relief of some of the most prevalent allergic diseases. In conclusion, this example shows that alkamides with fatty acids of medium length produced superior inhibition of mast cell activity, whereas alkamides with shorter and longer fatty acids were less inhibitory.

### REFERENCES CITED IN THIS EXAMPLE

Albin, K. C., Simons, C. T., 2010. Psychophysical Evaluation of a Sanshool Derivative (Alkylamide) and the Elucidation of Mechanisms Subserving Tingle. PLoS ONE 5(3), e9520.
Baba, Y., Hayashi, K., Fujii, Y., Mizushima, A., Watarai, H., Wakamori, M., Numaga, T., Mori, Y., lino, M., Hikida, M., 2006. Coupling of STIM1 to store-operated Ca2+ entry through its constitutive and inducible movement in the endoplasmic reticulum. Proceedings of the National Academy of Sciences 103(45), 16704-16709.
Baba, Y., Nishida, K., Fujii, Y., Hirano, T., Hikida, M., Kurosaki, T., 2008. Essential function for the calcium sensor STIM1 in mast cell activation and anaphylactic responses. Nat Immunol 9(1), 81-88.
Bauer, R., Remiger, P., Wagner, H., 1988. Alkamides from the roots of Echinacea purpurea. Phytochemistry 27(7), 2339-2342.
Bautista, D. M., Sigal, Y. M., Milstein, A. D., Garrison, J. L., Zorn, J. A., Tsuruda, P. R., Nicoll, R. A., Julius, D., 2008. Pungent agents from Szechuan peppers excite sensory neurons by inhibiting two-pore potassium channels. Nature Neuroscience 11(7), 772-779.
Boonen, J., Bronselaer, A., Nielandt, J., Veryser, L., De Tré, G., De Spiegeleer, B., 2012. Alkamid database: Chemistry, occurrence and functionality of plant N-alkylamides. Journal of Ethnopharmacology 142(3), 563-590.
Bryant, B. P., Mezine, I., 1999. Alkylamides that produce tingling paresthesia activate tactile and thermal trigeminal neurons. Brain Research 842(2), 452-460.
Cech, N. B., Eleazer, M. S., Shoffner, L. T., Crosswhite, M. R., Davis, A. C., Mortenson, A. M., 2006. High performance liquid chromatography/electrospray ionization mass spectrometry for simultaneous analysis of alkamides and caffeic acid derivatives from Echinacea purpurea extracts. J Chromatogr A 1103(2), 219-228.
Cech, N. B., Kandhi, V., Davis, J. M., Hamilton, A., Eads, D., Laster, S. M., 2010. Echinacea and its alkylamides: effects on the influenza A-induced secretion of cytokines, chemokines, and PGE(2) from RAW 264.7 macrophage-like cells. Int Immunopharmacol 10(10), 1268-1278.
Dedkova, E. N., Sigova, A. A., Zinchenko, V. P., 2000. Mechanism of action of calcium ionophores on intact cells: ionophore-resistant cells. Membrane & cell biology 13(3), 357-368.
Feske, S., 2009. ORAI1 and STIM1 deficiency in human and mice: roles of store-operated Ca2+ entry in the immune system and beyond. Immunological Reviews 231(1), 189-209.
Finn, D. F., Walsh, J. J., 2013. Twenty-first century mast cell stabilizers. British Journal of Pharmacology 170(1), 23-37.
Gilfillan, A. M., Tkaczyk, C., 2006. Integrated signalling pathways for mast-cell activation. Nat Rev Immunol 6(3), 218-230.
Graham, A. C., Hilmer, K. M., Zickovich, J. M., Obar, J. J., 2013. Inflammatory response of mast cells during influenza A virus infection is mediated by active infection and RIG-I signaling. The Journal of Immunology 190(9), 4676-4684.
Greger, H., 2016. Alkamides: a critical reconsideration of a multifunctional class of unsaturated fatty acid amides. Phytochemistry Reviews 15(5), 729-770.
Gronich, J. H., Bonventre, J. V., Nemenoff, R. A., 1990. Purification of a high-molecular-mass form of phospholipase A₂ from rat kidney activated at physiological calcium concentrations. Biochemical Journal 271(1), 37-43.
Hoth, M., Penner, R., 1992. Depletion of intracellular calcium stores activates a calcium current in mast cells. Nature 355(6358), 353-356.
Hou, C. C., Chen, C. H., Yang, N. S., Chen, Y. P., Lo, C. P., Wang, S. Y., Tien, Y. J., Tsai, P. W., Shyur, L. F., 2010. Comparative metabolomics approach coupled with cell- and gene-based assays for species classification and anti-inflammatory bioactivity validation of Echinacea plants. The Journal of Nutritional Biochemistry 21(11), 1045-1059.
Hou, C. C., Huang, C. C., Shyur, L. F., 2011. Echinacea Alkamides Prevent Lipopolysaccharide/D-Galactosamine-Induced Acute Hepatic Injury through JNK Pathway-Mediated HO-1 Expression. Journal of Agricultural and Food Chemistry 59(22), 11966-11974.
Kindscher, K., 1989. Ethnobotany of purple coneflower (Echinacea angustifolia, Asteraceae) and Other Echinacea Species. Econ Bot 43(4), 498-507.
Klein, M., Klein-Hessling, S., Palmetshofer, A., Serfling, E., Tertilt, C., Bopp, T., Heib, V., Becker, M., Taube, C., Schild, H., Schmitt, E., Stassen, M., 2006. Specific and Redundant Roles for NFAT Transcription Factors in the Expression of Mast Cell-Derived Cytokines. The Journal of Immunology 177(10), 6667-6674.
Kuehn, H. S., Radinger, M., Gilfillan, A. M., 2010. Measuring mast cell mediator release. Current Protocols in Immunology, 7.38. 31-37.38. 39.
LaLone, C. A., Hammer, K. D., Wu, L., Bae, J., Leyva, N., Liu, Y., Solco, A. K., Kraus, G. A., Murphy, P. A., Wurtele, E. S., 2007. Echinacea species and alkamides inhibit prostaglandin E2 production in RAW264. 7 mouse macrophage cells. Journal of Agricultural and Food Chemistry 55(18), 7314-7322.
Ley, J. P., Krammer, G., Looft, J., Reinders, G., Bertram, H., 2006. Structure-activity relationships of trigeminal effects for artificial and naturally occurring alkamides related to spilanthol. Developments in Food Science 43, 21.
Leyte-Lugo, M., Todd, D. A., Gulledge, T. V., Juzumaite, M., Carter, F. S., Laster, S. M., Cech, N. B., 2015. Cytokine-Suppressive Activity of a Hydroxylated Alkylamide from Echinacea purpurea. Planta Med Lett 2(01), e25-e27.
Liu, C., Au, J. D., Zou, H. L., Cotten, J. F., Yost, C. S., 2004. Potent Activation of the Human Tandem Pore Domain K Channel TRESK with Clinical Concentrations of Volatile Anesthetics. Anesthesia & Analgesia 99(6), 1715-1722.
Luik, R. M., Wu, M. M., Buchanan, J., Lewis, R. S., 2006. The elementary unit of store-operated Ca²⁺ entry: local activation of CRAC channels by STIM1 at ER-plasma membrane junctions. The Journal of Cell Biology 174(6), 815-825.
Moazami, Y., Gulledge, T. V., Laster, S. M., Pierce, J. G., 2015. Synthesis and biological evaluation of a series of fatty acid amides from Echinacea. Bioorganic & Medicinal Chemistry Letters 25(16), 3091-3094.
Olah, A., Szabo-Papp, J., Soeberdt, M., Knie, U., Dahnhardt-Pfeiffer, S., Abels, C., Biro, T., 2017. Echinacea purpurea-derived alkylamides exhibit potent anti-inflammatory effects and alleviate clinical symptoms of atopic eczema. J Dermatol Sci.
Ottea, J. A., Payne, G. T., Soderlund, D. M., 1990. Action of insecticidal N-alkylamides at site 2 of the voltage-sensitive sodium channel. Journal of Agricultural and Food Chemistry 38(8), 1724-1728.
Passante, E., Frankish, N., 2009. The RBL-2H3 cell line: its provenance and suitability as a model for the mast cell. Inflammation Research 58(11), 737-745.
Pawankar, R., Canonica, G., Holgate, S., Lockey, R., 2013. The World Allergy Organization (WAO) White Book on Allergy (Update).
Raduner, S., Majewska, A., Chen, J. Z., Xie, X. Q., Hamon, J., Faller, B., Altmann, K. H., Gertsch, J., 2006. Alkylamides from Echinacea are a new class of cannabinomimetics Cannabinoid type 2 receptor-dependent and -independent immunomodulatory effects. Journal of Biological Chemistry 281(20), 14192-14206.
Rivera, J., Fierro, N. A., Olivera, A., Suzuki, R., 2008. New Insights on Mast Cell Activation via the High Affinity Receptor for IgE1, Advances in Immunology. Academic Press, pp. 85-120.
Sharma, M., Anderson, S. A., Schoop, R., Hudson, J. B., 2009. Induction of multiple pro-inflammatory cytokines by respiratory viruses and reversal by standardized Echinacea, a potent antiviral herbal extract. Antiviral Research 83(2), 165-170.
Smyth, J. T., DeHaven, W. I., Bird, G. S., Putney, J. W., 2007. Role of the microtubule cytoskeleton in the function of the store-operated Ca²⁺ channel activator STIM1. Journal of Cell Science 120(21), 3762-3771.
Spelman, K., Iiams-Hauser, K., Cech, N. B., Taylor, E. W., Smirnoff, N., Wenner, C. A., 2009. Role for PPARγ in IL-2 inhibition in T cells by Echinacea-derived undeca-2E-ene-8,10-diynoic acid isobutylamide. Int Immunopharmacol 9(11), 1260-1264.
Sugai, E., Morimitsu, Y., Iwasaki, Y., Morita, A., Watanabe, T., Kubota, K., 2005. Pungent Qualities of Sanshool-Related Compounds Evaluated by a Sensory Test and Activation of Rat TRPV1. Bioscience, Biotechnology, and Biochemistry 69(10), 1951-1957.
Sutovska, M., Capek, P., Kazimierova, I., Pappova, L., Joskova, M., Matulova, M., Franova, S., Pawlaczyk, I., Gancarz, R., 2015. Echinacea complex-chemical view and anti-asthmatic profile. J Ethnopharmacol 175, 163-171.
Talley, E. M., Bayliss, D. A., 2002. Modulation of TASK-1 (Kcnk3) and TASK-3 (Kcnk9) Potassium Channels: Volatile Anesthetics and Neurotransmitters Share a Molecular Site of Action. Journal of Biological Chemistry 277(20), 17733-17742.
Todd, D. A., Gulledge, T. V., Britton, E. R., Oberhofer, M., Leyte-Lugo, M., Moody, A. N., Shymanovich, T., Grubbs, L. F., Juzumaite, M., Graf, T. N., Oberlies, N. H., Faeth, S. H., Laster, S. M., Cech, N. B., 2015. Ethanolic Echinacea purpurea Extracts Contain a Mixture of Cytokine-Suppressive and Cytokine-Inducing Compounds, Including Some That Originate from Endophytic Bacteria. PLoS ONE 10(5), e0124276.
Tsien, R. Y., 1980. New calcium indicators and buffers with high selectivity against magnesium and protons: design, synthesis, and properties of prototype structures. Biochemistry 19(11), 2396-2404.
Tsunozaki, M., Lennertz, R. C., Vilceanu, D., Katta, S., Stucky, C. L., Bautista, D. M., 2013. A ‘toothache tree’alkylamide inhibits A6 mechanonociceptors to alleviate mechanical pain. The Journal of physiology 591(13), 3325-3340.
Vig, M., DeHaven, W. I., Bird, G. S., Billingsley, J. M., Wang, H., Rao, P. E., Hutchings, A. B., Jouvin, M.-H., Putney, J. W., Kinet, J.-P., 2008. Defective mast cell effector functions in mice lacking the CRACM1 pore subunit of store-operated calcium release-activated calcium channels. Nat Immunol 9(1), 89-96.
Vig, M., Kinet, J.-P., 2009. Calcium signaling in immune cells. Nat Immunol 10(1), 21-27.
Wernersson, S., Pejler, G., 2014. Mast cell secretory granules: armed for battle. Nat Rev Immunol 14(7), 478-494.
Woelkart, K., Bauer, R., 2007. The Role of Alkamides as an Active Principle of Echinacea. Planta Med 73(07), 615-623.
Woelkart, K., Linde, K., Bauer, R., 2008. Echinacea for Preventing and Treating the Common Cold. Planta Med 74(06), 633-637.

### Example 4. Synthesis of Alkamides

Nonlimiting examples of compounds of Formula I, II, or III include:

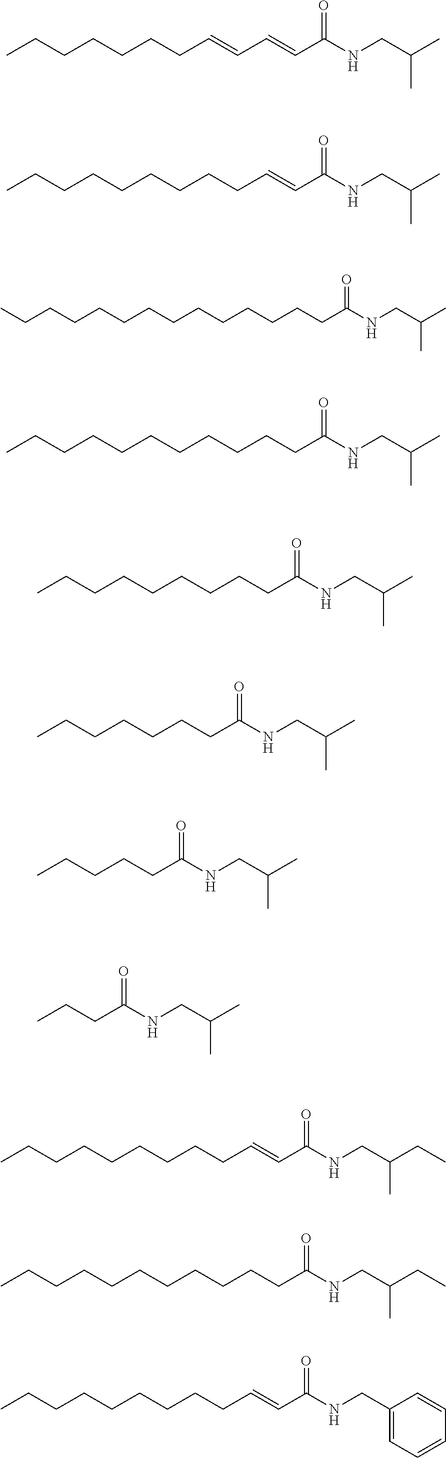

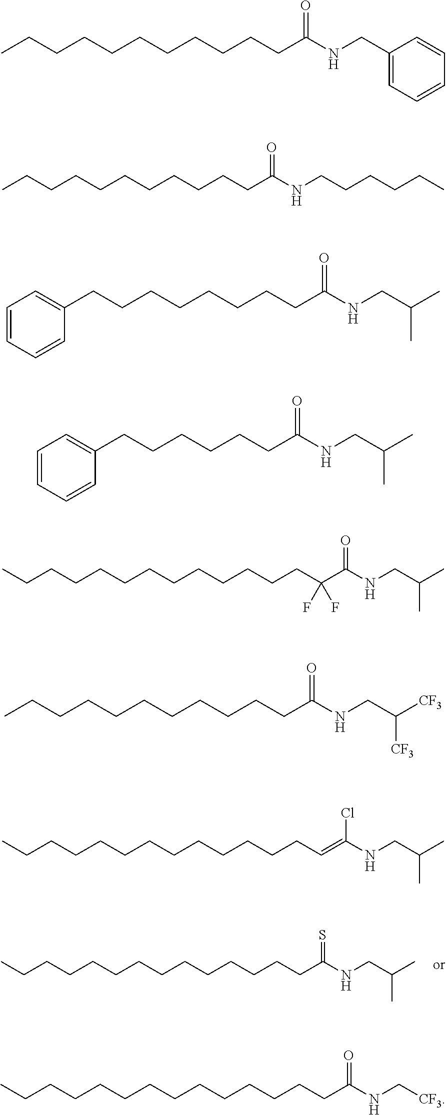

### Chemical Synthesis

All reactions were performed under an Ar atmosphere and all glassware were dried in an oven at 135° C. overnight prior to use, unless otherwise noted. DCM was purified using an alumina filtration system. (2E,4E)-dodeca-2,4-dien-1-ol, lauric acid, other carboxylic derivatives, and propylphosphonic anhydride solution (T3P) were purchased from Aldrich and Fisher Scientific and used as received unless otherwise noted.

Reactions were monitored by TLC analysis (EM Science pre-coated silica gel 60 F₂₅₄ plates, 250 mm layer thickness) and visualization was accomplished with a 254 nm UV light and by staining with a KMnO₄ solution (1.5 g of KMnO₄, 10 g of K₂CO₃, and 1.25 mL of a 10% NaOH solution in 200 mL of water). Reactions were also monitored by LC-MS (Shimadzu LC-MS 2020 with Kinetex 2.6 mm C18 50×2.10 mm). Flash chromatography on SiO₂ was used to purify the crude reaction mixtures and performed on a Biotage Isolera utilizing Biotage cartridges and linear gradients.

¹H and ¹³C NMR spectra were obtained on a Varian Mercury-VX 300, a Varian Mercury-VX 400, or a Varian Mercury-Plus 300 instrument in CDCl₃ unless otherwise noted. Chemical shifts were reported in parts per million with the residual solvent peak used as an internal standard (CDCl3 ¹H δ=7.26 and ¹³C δ=77.23). ¹H NMR spectra were run at 300 or 400 MHz and are tabulated as follows: chemical shift, multiplicity (s=singlet, d=doublet, t=triplet, q=quartet, m=multiplet, brs=broad singlet, dd=doublet of doublet, dt=doublet of triplet, p=pentet), number of protons, and coupling constant(s). ¹³C NMR spectra were run at 100 MHz using a proton-decoupled pulse sequence with a d₁ of 0 second unless otherwise noted, and are tabulated by observed peak. High-resolution mass spectra were obtained on a Thermo Fisher Scientific, Exactive Plus mass spectrometer (Ion trap) using Heated Electrospray Ionization (HESI).

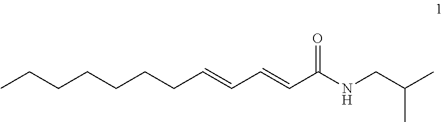 [SMILES: CCCCCCC/C=C/C=C/C(=O)NCC(C)C]

(2E,4E)-N-Isobutyldodeca-2,4-dienamide (1). General protocol A. To a solution of (2E,4E)-dodeca-2,4-diezoic acid 3 (0.160 g, 0.783 mmol) in anhydrous DCM (8.00 mL) was added Et₃N (0.317 g, 3.13 mmol) and T3P® (0.598 g, 0.939 mmol) at rt and the mixture was stirred for 20 min. Isobutylamine (57.8 mg, 78.3 mmol) was added drop-wise and the mixture was stirred overnight. The solvent was removed in vacuo and the residue was purified by chromatography on SiO₂ (hexanes/EtOAc, 0-100%) to yield 97.0 mg (49%) of 1 as a light yellow solid: ¹H NMR (400 MHz, CDCl₃) δδ 7.19 (dd, 1H, J=14.9, 9.9 Hz), 6.16-6.02 (m, 2H), 5.75 (d, 1H, J=15.1 Hz), 5.51 (brs, 1H), 3.16 (t, 2H, J=6.5 Hz), 2.14 (q, 2 H, J=7.5, 7.1 Hz), 1.83-1.34 (m, 1H), 1.45-1.34 (m, 2H), 1.33-1.20 (m, 8H), 0.92 (d, 6H, J=6.7 Hz), 0.87 (3 H, J=6.8 Hz); ¹³C NMR (100 MHz, CDCl₃) δ 166.6, 143.6, 141.5, 128.3, 121.8, 47.1, 33.2, 32.0, 29.3, 28.8, 22.9, 20.3, 14.3; ESIMS m/z 293 [M+MeCN]⁺; HRMS m/z calculated for C₁₆H₃₀ON [M+H]⁺ 252.2322, found 252.2318.

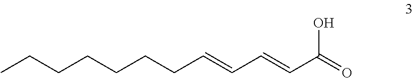

(2E,4E)-Dodeca-2,4-dienoic acid (3). To a solution of (2E,4E)-dodeca-2,4-dien-1-ol 2 (0.236 g, 1.24 mmol) in DCM (4.80 mL) was added activated Mn(IV)O₂ (0.336 g, 3.86 mmol). The reaction mixture was stirred for 48 h and filtered through a pad of Celite®. The filtrate was concentrated in vacuo to afford the crude aldehyde. To a solution of the crude aldehyde (0.181 g, 0.962 mmol) and 2-methyl-2-butene (0.675 g, 9.62 mmol) in t-BuOH (10.0 mL) and water (10.0 mL) at 0° C. was added a solution of sodium chlorite (0.218 g, 1.92 mmol) and monobasic sodium phosphate (0.233 g, 1.92 mmol) in water (6.40 mL). The reaction was allowed to warm to rt and stirred for 1 h. The mixture was poured into water and diluted with EtOAc. The aqueous layer was extracted with EtOAc and the combined organic layers were dried (MgSO4), and concentrated in vacuo to afford 0.160 g (85%) of 3 which was used in the subsequent step without further purification: ¹H NMR (300 MHz, CDCl₃) δ 7.39-7.26 (m, 1H), 6.22-6.19 (m, 2H), 5.80 (d, 1H, J=15.2 Hz), 2.23-2.13 (m, 2 H), 1.48-1.36 (m, 2H), 1.35-1.19 (m, 10H), 0.87 (t, 3H, J=6.9 Hz); ESIMS m/z 196 [M−H]⁻;

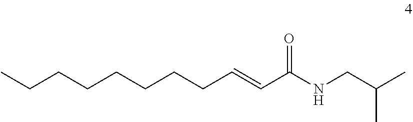 [SMILES: CCCCCCCC/C=C/C(=O)NCC(C)C]

(E)-N-Isobutyldodec-2-enamide (4). According to general protocol A, (E)-dodec-2-enoic acid¹ (0.314 g, 1.57 mmol), Et₃N (0.635 g, 6.28 mmol), T3P® (0.999 g, 1.57 mmol) and isobutylamine (0.116, 1.57 mmol) in anhydrous DCM (15.0 mL) for 16 h afforded 87.5 mg (22%) of 4 as a light yellow solid after purification by chromatography on SiO₂ (hexanes/EtOAc, 1 to 100%): ¹H NMR (400 MHz, CDCl₃) δ□6.83 (dt, 1 H, J=15.1, 6.9 Hz), 5.75 (dt, 1 H, J=15.2, 1.5 Hz), 5.45 (brs, 1H), 3.15 (t, 2H, J=6.8 Hz), 2.19-2.13 (m, 2H), 1.75-1.81 (m, 1H), 1.45-1.39 (m, 2H), 1.28-1.23 (m, 12H), 0.92 (d, 6H, J=6.7 Hz), 0.87 (t, 3H, J=6.9 Hz); ¹³C NMR (100 MHz, CDCl₃) δ 166.3, 145.1, 123.7, 47.0, 32.3, 32.1, 29.8, 29.7, 29.7, 29.5, 29.4, 28.8, 28.5, 22.9, 20.3, 14.3; ESIMS m/z 295 [M+H]⁺; HRMS m/z calculated for C₁₆H₃₂NO [M+H]⁺ 254.2478, found 254.2472.

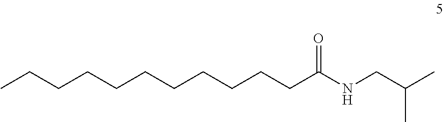 [SMILES: CCCCCCCCCCCC(=O)NCC(C)C]

N-Isobutyldodecanamide (5). According to general protocol A, lauric acid (0.881 g, 4.35 mmol), Et₃N (1.76 g, 17.4 mmol), T3P® (3.23 g, 5.22 mmol) and isobutylamine (0.318 g, 4.35 mmol) in anhydrous DCM (43.0 mL) for 16 h afforded 1.01 g (91%) of 5 as a white solid after purification by chromatography on SiO₂ (hexanes/EtOAc, 1 to 100%): ¹H NMR (400 MHz, CDCl3) δ□5.44 (brs, 1H), 3.08 (t, 2H, J=6.4 Hz), 2.17 (t, 2H, J=8.0 Hz), 1.80-1.70 (m, 1H), 1.63-1.59 (m, 2H), 1.29-1.24 (m, 16H), 0.91-0.87 (m, 9H); ¹³C NMR (100 MHz, CDCl₃) δ 173.4, 47.0, 37.2, 37.2, 32.1, 29.7, 29.6, 28.7, 26.2, 23.0, 20.3, 14.4; ESIMS m/z 297 [M+ACN]⁺; HRMS m/z calculated for C₁₆H₃₄NO [M+H]⁺ 256.2635, found 256.2630.

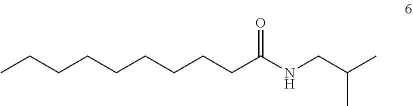 [SMILES: CCCCCCCCCC(=O)NCC(C)C]

N-Isobutyldecanamide (6). According to general protocol A, decanoic acid (0.300 g, 1.72 mmol), Et₃N (0.698 g, 6.90 mmol), T3P® (1.32 g, 2.07 mmol) and isobutylamine (0.127 g, 1.72 mmol) in anhydrous DCM (17.0 mL) stirred overnight afforded 0.319 g (81%) of 6 as a white solid after purification by chromatography on SiO₂ (hexanes/EtOAc, 5 to 100%): ¹H NMR (400 MHz, CDCl3) δ 5.45 (brs, 1H), 3.08 (t, 2H, J=6.4 Hz), 2.16 (t, 2H, J=8.0 Hz), 1.79-1.72 (m, 2H), 1.64-1.60 (m, 2H), 1.36-1.17 (m, 12H), 0.91 (d, 6H, J=6.7 Hz), 0.87 (t, 3H, J=7.2 Hz); ¹³C NMR (100 MHz, CDCl3) δ 173.5, 47.0, 37.2, 34.1, 31.9, 31.9, 29.5, 29.2, 29.1, 28.7, 26.1, 25.0; ESIMS m/z 269 [M+ACN]⁺; HRMS m/z calculated for C₁₄H₃₀NO [M+H]⁺ 228.2322, 228.2316.

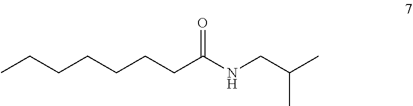

N-Isobutyloctanamide (7). According to general protocol A, octanoic acid (0.300 g, 2.06 mmol), Et₃N (0.317 g, 3.13 mmol), T3P® (0.598 g, 0.939 mmol) and isobutylamine (57.8 mg, 0.783 mmol) in anhydrous DCM (20.0 mL) stirred overnight afforded 0.311 g (84%) of 7 as a colorless oil after purification by chromatography on SiO₂ (hexanes/EtOAc, 2 to 100%): ¹H NMR (300 MHz, CDCl3) δ 5.45 (brs, 1H), 3.08 (t, 2H, J=6.5 Hz), 2.16 (t, 2H, J=8.1 Hz), 1.79-1.72 (m, 2H), 1.64-1.60 (m, 2H), 1.36-1.17 (m, 12H), 0.91 (d, 6H, J=6.7 Hz), 0.87 (t, 3H, J=6.9 Hz); ¹³C NMR (100 MHz, CDCl3) δ 173.4, 94.7, 47.9, 47.0, 44.7, 37.2, 31.9, 29.5, 29.2, 28.7, 28.7, 26.1, 22.8, 20.3, 20.3, 14.3, 14.0; ESIMS m/z 241 [M+MeCN]⁺; HRMS m/z calculated for C₁₂H₂₆ON [M+H]⁺ 200.2009, found 200.2006.

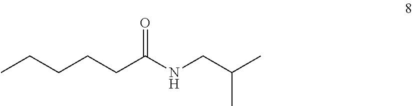

N-Isobutylhexanamide (8). According to general protocol A, hexanoic acid (1.00 g, 8.52 mmol), Et₃N (3.96 g, 34.1 mmol), T3P® (6.51 g, 10.2 mmol) and isobutylamine (0.623 g, 8.52 mmol) in anhydrous DCM (85.0 mL) for 16 h afforded 0.680 g (47%) of 5 as a light yellow oil after purification by chromatography on SiO₂ (hexanes/EtOAc, 1 to 100%): ¹H NMR (400 MHz, CDCl3) δ□5.51 (brs, 1H), 3.07 (t, 2H, J=6.0 Hz), 2.16 (t, 2H, J=7.6 Hz), 1.80-1.70 (m, 1H), 1.66-1.59 (m, 2H), 1.35-1.25 (m, 4H), 0.91-0.87 (m, 9H); ¹³C NMR (CDCl3, 100 MHz) δ 173.3, 47.0, 37.2, 31.7, 28.8, 25.8, 22.6, 20.3, 14.2; ESIMS m/z 213 [M+MeCN]⁺; HRMS m/z calculated for C₁₀H₂₂ON [M+H]⁺ 172.1696, found 172.1696.

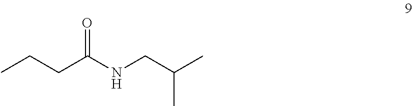

N-Isobutylbutyramide (9). According to general protocol A, butyric acid (0.300 g, 3.40 mmol), Et₃N (1.38 g, 13.6 mmol), T3P® (2.60 g, 4.09 mmol) and isobutylamine (0.252 g, 3.40 mmol) in anhydrous DCM (35.0 mL) for 16 h afforded 0.389 g (80%) of 5 as a colorless oil after purification by chromatography on SiO₂ (hexanes/EtOAc, 1 to 100%): ¹H NMR (300 MHz, CDCl3) δ□5.56 (brs, 1H), 3.08 (t, 2H, J=6.5 Hz), 2.15 (t, 2H, J=7.8 Hz), 1.80-1.65 (m, 3H), 0.97 (t, 3H, J=7.5 Hz), 0.91 (d, 6H, J=6.7 Hz); ¹³C NMR (CDCl₃, 100 MHz) δ 173.2, 47.0, 39.1, 28.7, 20.2, 19.5, 14.0; ESIMS m/z 213 [M+MeCN]⁺; HRMS m/z calculated for C₈H₁₉NO [M+H]⁺ 144.1383, found 144.1381.

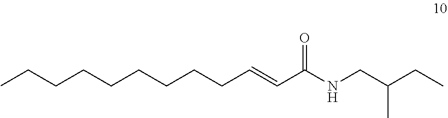 [SMILES: CCCCCCCCC/C=C/C(=O)NCC(C)CC]

(E)-N-(2-Methylbutyl)dodec-2-enamide (10). According to general protocol A, (E)-dodec-2-enoic acid¹ (0.400 g, 2.00 mmol), Et₃N (0.810 g, 8.00 mmol), T3P® (1.53 g, 2.40 mmol) and 2-methylbutylamine (0.205 g, 2.00 mmol) in anhydrous DCM (20.0 mL) for 16 h afforded 0.150 g (28%) of 10 as a light yellow solid after purification by chromatography on SiO₂ (hexanes/EtOAc. 1 to 100%): ¹H NMR (400 MHz, CDCl3) δ□6.83 (dt, 1 H, J=15.3, 6.9 Hz), 5.75 (dt, 1 H, J=15.2, 1.5 Hz), 5.43 (brs, 1H), 3.32-3.22 (m, 1H), 3.18-2.98 (m, 1H), 2.20-2.12 (m, 2H), 1.65-1.51 (m, 2H), 1.45-1.39 (m, 4H), 1.28-1.23 (m or s, 12H), 1.20-1.10 (m, 2H), 0.93-0.87 (m, 9H); ¹³C NMR (100 MHz, CDCl₃) δ 166.3, 145.0, 123.7, 45.3, 35.2, 32.2, 32.1, 29.8, 29.7, 29.5, 29.4, 28.5, 27.2, 22.9, 17.5, 14.3, 11.5; ESIMS m/z 309 [M+MeCN]⁺.

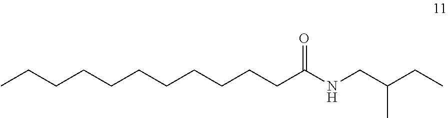 [SMILES: CCCCCCCCCCCC(=O)NCC(C)CC]

N-(2-Methylbutyl)dodecanamide (11). According to general protocol A, lauric acid (0.720 g, 3.53 mmol), Et₃N (1.43 g, 14.1 mmol), T3P® (2.70 g, 4.24 mmol) and 2-methylbutylamine (0.362 g, 3.53 mmol) in anhydrous DCM (35.0 mL) stirred overnight afforded 0.92 g (97%) of 11 as a white solid after purification by chromatography on SiO₂ (hexanes/EtOAc, 0-5 or 100%):
¹H NMR (400 MHz, CDCl₃) δ 5.39 (brs, 1H), 3.24-3.18 (m, 1H), 3.09-3.03 (m, 1H), 2.17 (t, 2H, J=8.0 Hz), 1.62-1.51 (m, 3H), 1.48-1.37 (m, 2H), 1.35-1.25 (m, 12H), 1.17-1.07 (m, 2H), 0.93-0.85 (m, 9H); ¹³C NMR (100 MHz, CDCl3) δ 173.3, 45.3, 38.0, 37.2, 35.2, 32.1, 29.8, 27.2, 26.1, 22.9, 17.4, 14.4, 11.5; ESIMS m/z 311 [M+MeCN]⁺; HRMS m/z calculated for C₁₇H₃₆NO [M+H]⁺ 270.2791, found 270.2785.

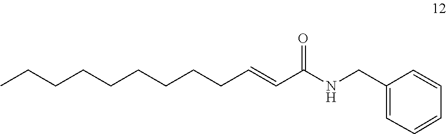 [SMILES: CCCCCCCCC/C=C/C(=O)NCc1ccccc1]

(E)-N-Benzyldodec-2-enamide (12). According to general protocol A, (E)-dodec-2-enoic acid 1(0.366 g, 1.83 mmol), Et₃N (0.20 mL, 1.83 mmol), T3P® (1.31 mL, 2.19 mmol) and benzylamine (0.198, 1.83 mmol) in anhydrous DCM (18.3 mL) for 16 h afforded 0.173 g (33%) of 12 as a white solid after purification by chromatography on SiO₂ (hexanes/EtOAc, 1 to 100%): ¹H NMR (400 MHz, CDCl₃) δ 7.36-7.26 (m, 5H), 6.88 (dt, 1 H, J=15.2, 6.9 Hz), 5.77 (dt, 1 H, J=15.2, 1.5 Hz), 5.71 (brs, 1H), 4.51 (d, 2H, J=5.7 Hz), 2.17 (q, 2 H, J=7.1 Hz), 1.47-1.40 (m, 2H), 1.40-1.34 (m, 12H), 0.87 (t, 6H, J=6.8 Hz), 0.88 (t, 3H, J=8.0 Hz); ¹³C NMR (100 MHz, CDCl₃) δ 166.3, 145.1, 123.7, 47.0, 32.3, 32.1, 29.8, 29.7, 29.7, 29.5, 29, 4, 28.8, 28.5, 22.9, 20.3, 14.3; ESIMS m/z 329 [M+MeCN]⁺; HRMS m/z calculated for C₁₉H₃₀NO [M+H]⁺ 288.2322, found 288.2317.

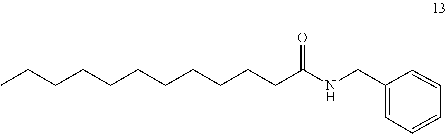 [SMILES: CCCCCCCCCCCC(=O)NCc1ccccc1]

(E)-N-Benzyldodec-2-enamide (13). According to general protocol A, lauric acid (0.772 g, 3.82 mmol), Et₃N (1.54 g, 15.3 mmol), T3P® (2.91 g, 4.58 mmol) and benzylamine (0.413 g, 3.82 mmol) in anhydrous DCM (38.0 mL) for 16 h afforded 0.94 g (85%) of 13 as a white solid after purification by chromatography on SiO₂ (hexanes/EtOAc, 1 to 100%): ¹H NMR (400 MHz, CDCl₃) δ□5.68 (brs, 1H), 4.45 (d, 2H, J=5.5 Hz), 2.21 (t, 2H, J=7.7 Hz), 1.65 (p, 2 H, J=7.5 Hz), 1.29-1.24 (m, 16H), 0.87 (t, 3H, J=4.8 Hz); ¹³C NMR (100 MHz, CDCl₃) δ 173.2, 138.6, 128.9, 128.1, 127.7, 94.5, 43.8, 37.1, 32.1, 29.8, 29.7, 29.5 26.0, 22.9, 14.5; ESIMS m/z 331 [M+MeCN]⁺; HRMS m/z calculated for C₁₉H₃₂NO [M+H]⁺ 290.2478, found 290.2473.

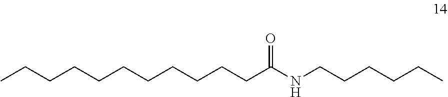 [SMILES: CCCCCCCCCCCC(=O)NCCCCCC]

N-Hexyldodecanamide (14). According to general protocol A, lauric acid (0.881 g, 4.35 mmol), Et₃N (1.76 g, 17.4 mmol), T3P® (3.23 g, 5.22 mmol) and hexylamine (0.440 g, 4.35 mmol) in anhydrous DCM (43.0 mL) for 16 afforded 0.788 mg (64%) of 24 as a white solid after purification by chromatography on SiO₂ (hexanes/EtOAc, 1 to 100%): ¹H NMR (400 MHz, CDCl₃) δ□5.39 (brs, 1H), 3.26-2.21 (m, 2H), 2.14 (t, 2H, J=8.0 Hz), 1.66-1.62 (m, 2H), 1.52-1.43 (m, 2H), 1.34-1.20 (m, 22H), 0.89-0.86 (m, 6H); ¹³C NMR (100 MHz, CDCl₃) δ 173.2, 39.7, 37.2, 32.1, 31.7, 29.7, 29.6, 26.8, 26.1, 22.9, 22.8, 14.4, 14.2; ESIMS m/z 284.50 [M+H]⁺; HRMS m/z calculated for C₁₈H₃₈ON [M+H]⁺ 284.2948, found 284.2943.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method of treating pain or itch, comprising administering to a subject in need thereof, a therapeutically effective amount of a compound of Formula I: 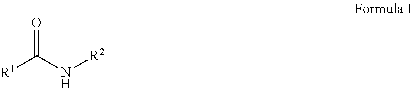
wherein:
R¹ and R² are independently selected from unsubstituted alkyl or substituted alkyl, unsubstituted alkenyl or substituted alkenyl, unsubstituted arylalkyl or substituted arylalkyl;
or a pharmaceutically acceptable salt thereof.

2. The method of claim 1, wherein R¹ is unsubstituted alkyl.

3. The method of claim 1, wherein R¹ is an unbranched alkyl.

4. The method of claim 1, wherein R¹ is a C₁₁alkyl.

5. The method of claim 1, wherein R¹ is a haloalkyl.

6. The method of claim 1, wherein R² is a branched alkyl.

7. The method of claim 1, wherein R² is isobutyl.

8. The method of claim 1, wherein R² is a haloalkyl.

9. The method of claim 1, wherein R¹ is selected from unsubstituted arylalkyl and R² is selected from unsubstituted alkyl or substituted alkyl.

10. The method of claim 1, wherein R¹ is selected from unsubstituted alkyl or substituted alkyl and R² is selected from substituted alkyl.

11. The method of claim 1, wherein the compound is formulated for topical delivery.

12. The method of claim 1, wherein the compound is selected from the following: 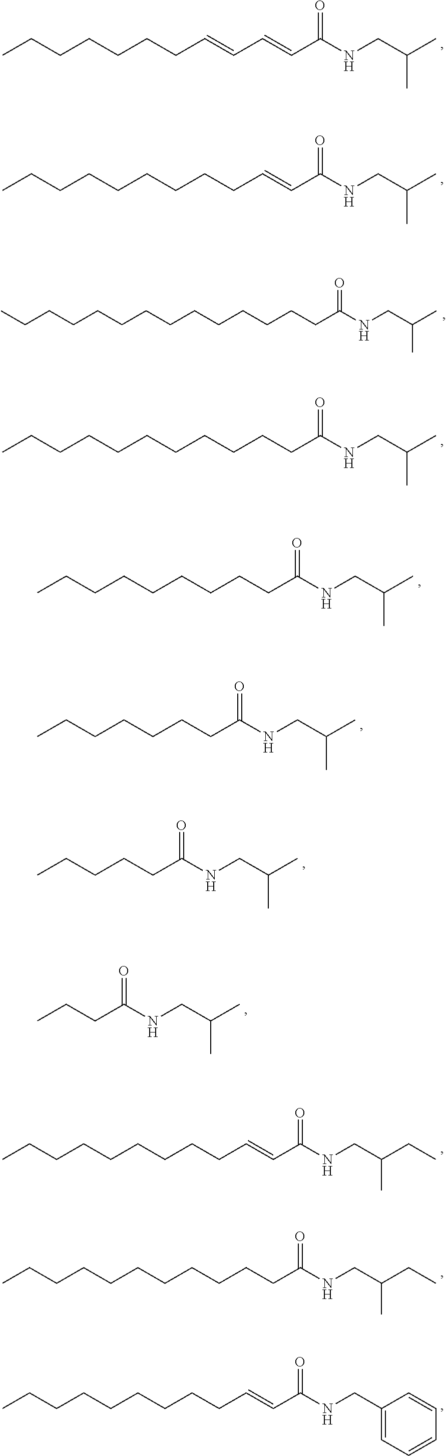 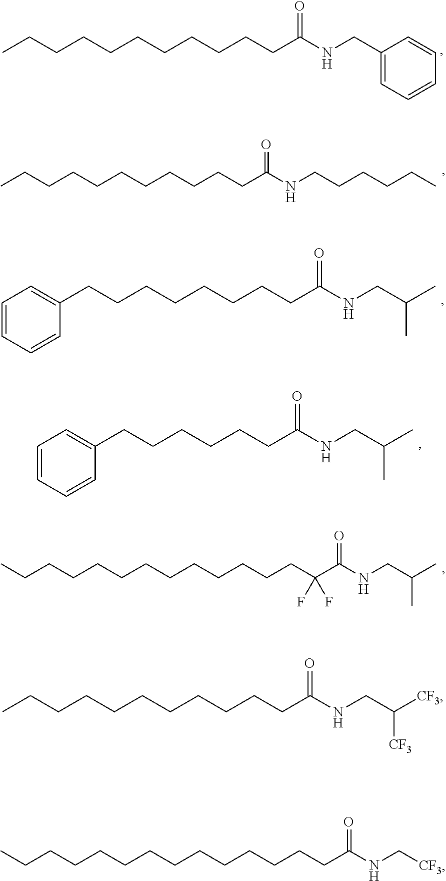
or a pharmaceutically acceptable salt thereof.

13. The method of claim 12, wherein the compound is: 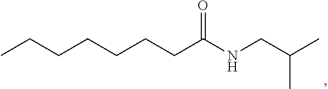
or a pharmaceutically acceptable salt thereof.

14. A method of treating an allergic disease, comprising administering to a subject in need thereof, a therapeutically effective amount of a compound of Formula I: 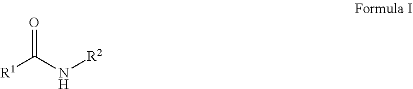
wherein:
R¹ and R² are independently selected from unsubstituted alkyl or substituted alkyl, unsubstituted alkenyl or substituted alkenyl, unsubstituted arylalkyl or substituted arylalkyl;
or a pharmaceutically acceptable salt thereof.

15.-23. (canceled)

24. The method of claim 14, wherein the compound is formulated for topical delivery.

25. The method of claim 14, wherein the allergic disease is selected from a hypersensitive reaction of the skin to a metal, an allergic response to pollen, an allergic response to pet dander, eczema, urticaria, asthma, or a food allergy.

26. The method of claim 25, wherein the allergic disease is urticaria.

27. The method of claim 14, wherein the compound is selected from the following: 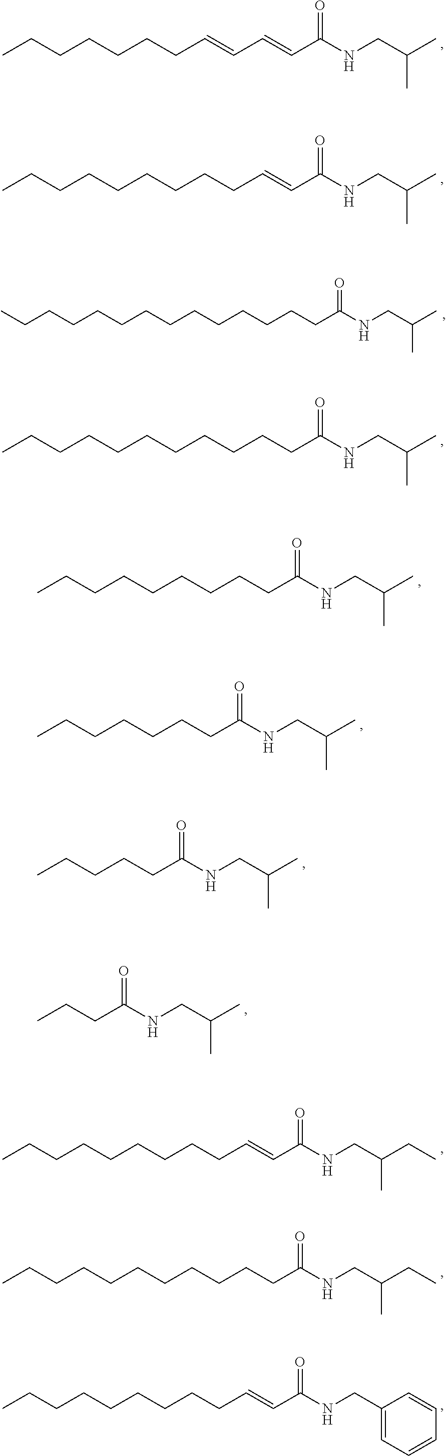 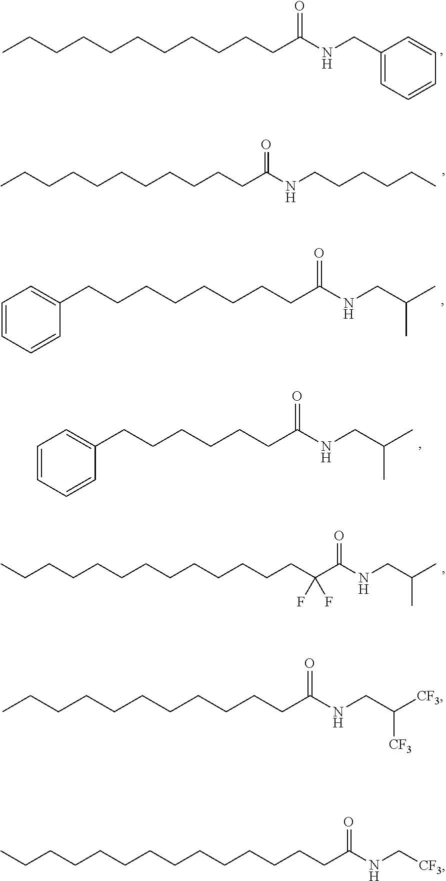
or a pharmaceutically acceptable salt thereof.

28. The method of claim 27, wherein the compound is: 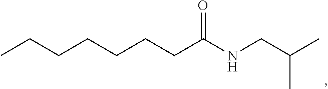
or a pharmaceutically acceptable salt thereof.

29.-42. (canceled)

43. A compound selected from the following: 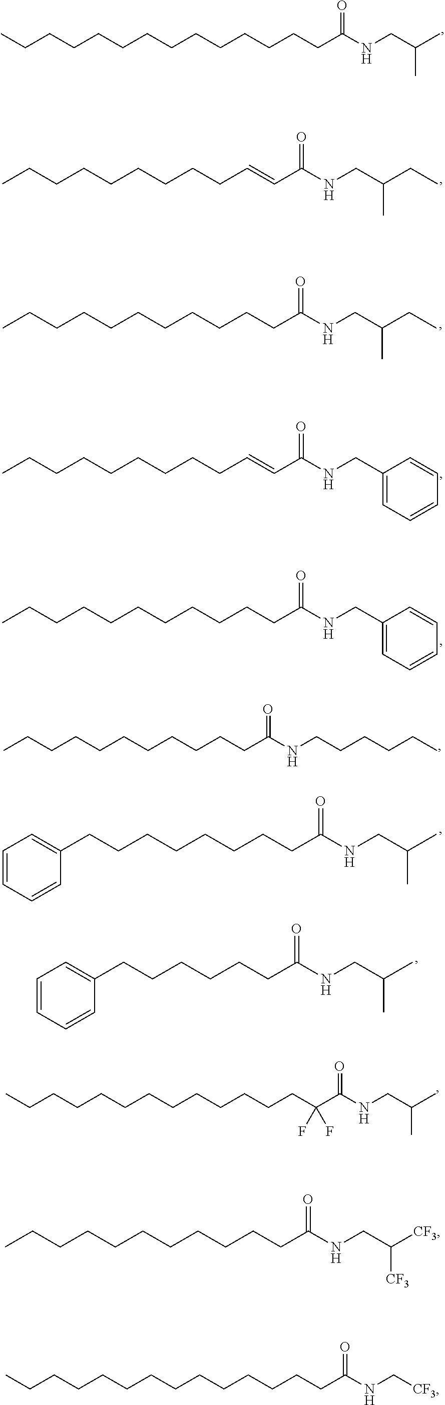
or a pharmaceutically acceptable salt thereof.
